(19) United States
(12) Patent Application Publication
(10) Pub. No.: US 2012/0196933 A1
(43) Pub. Date: Aug. 2, 2012

(54) MEXILETINE PRODRUGS

(76) Inventors: Richard Franklin, Hampshire (GB); Bernard T. Golding, Newcastle upon Tyne (GB); Robert G. Tyson, Durham (GB)

(21) Appl. No.: 13/335,914

(22) Filed: Dec. 22, 2011

Related U.S. Application Data
(60) Provisional application No. 61/426,980, filed on Dec. 23, 2010.

(30) Foreign Application Priority Data
Dec. 23, 2010 (GB) 1021891.5
Jul. 4, 2011 (GB) 1111379.2

(51) Int. Cl. A61K 31/205; C07C 279/12; A61P 25/00; C07C 317/50; A61P 21/00; C07C 237/20; A61K 31/165

(52) U.S. Cl. 514/554; 562/448; 564/196; 514/626; 562/430

(57) ABSTRACT

The present invention concerns prodrugs of mexiletine (and mexiletine's active metabolite) pharmaceutical compositions containing such prodrugs. Methods for treating myotonic conditions, while reducing the inherent adverse GI side effects associated with mexiletine, increasing the bioavailability of mexiletine, and improving the pharmacokinetic reproducibility of mexiletine with the aforementioned prodrugs are also provided.

This application claims priority to U.S. Provisional Application No. 61/426,980, filed Dec. 23, 2010; Great Britain Provisional Application No. GB 1021891.5, filed Dec. 23, 2010; and Great Britain Provisional Application No. 1111379.2, filed Jul. 4, 2011. The contents of these applications are hereby incorporated by reference in their entirety.

### FIELD OF THE INVENTION

The present invention relates to various prodrugs of mexiletine and pharmaceutically acceptable salts thereof and their use in the treatment of muscle myotonias and dystonia and neuropathic pain.

### BACKGROUND OF THE INVENTION

Myotonia is an abnormal delay in the relaxation of muscles after contraction. It is a key symptom in a number of muscle diseases called myotonic disorders. It can be mild or severe, interfering with daily activities such as walking, climbing stairs or opening and closing the eyelids. It can be worse after periods of rest or triggered by cold but improves after the muscles have warmed-up. However, prolonged, rigorous exercise may also trigger the condition. Individuals with the disorder may have trouble releasing their grip on objects or may have difficulty rising from a sitting position and a stiff, awkward gait.

It may be acquired or inherited, and is caused by an abnormality in the muscle membrane, specifically, the ion channels that control the contraction of muscle fibres.

Myotonia is a symptom commonly seen in patients with myotonic muscular dystrophy, and in a group of disorders called channelopathies (hereditary diseases that are caused by mutations in the chloride, sodium or potassium ion transport channels in the muscle membrane), such as Myotonia Congenita (Congenital Myotonia) of which two types called Becker's Disease and Thomsen's Disease exist.

Myotonia can affect all muscle groups; however the pattern of affected muscles can vary depending on the specific disorder involved.

People suffering from disorders involving myotonia can have a life threatening reaction to certain anaesthetics, one of these conditions occurs when the patient is under anaesthetic and is termed “Malignant hyperthermia”.

While people with mild myotonia can manage their disease without medication, more severe cases require drug treatment. Drugs that have been used to treat myotonia include sodium channel blockers such as procainamide, phenyloin and mexiletine, tricyclic antidepressant drugs such as clomipramine or imipramine, benzodiazepines, calcium antagonists, taurine and prednisone. However, each of these has their limitations in terms of efficacy and safety.

A related condition, dystonia, is a common neurological movement disorder characterised by sustained and involuntary muscle contractions or muscle spasms. These spasms can cause twisting, repetitive movements or abnormal postures and are sometimes accompanied by tremor. It is estimated that there are at least 70,000 people living with dystonia in the UK. The condition affects males and females of all ages.

Neuropathic pain is estimated to impact between 2.8 and 4.7% of the global population (Neuropathic Pain Network and Pfizer Inc., 2006 survey). Broadly classified as central or peripheral, neuropathic pain is caused by injury to, or disease of, the nervous system, or pain derived from damage to the nervous system itself, rather than pain detected by the nervous system due to external stimuli such as burns or broken limbs. Central neuropathic pain occurs as a result of damage to the central nervous system (CNS), and can be caused by, for example, multiple sclerosis, spinal cord injury, stroke or cancer. Peripheral neuropathic pain arises from damage to the peripheral nervous system caused by diabetes, cancer, HIV infection, carpel tunnel syndrome and post hepatic neuralgia, amputation (phantom limb pain), back injury, leg ulcers and iatrogenic injury through surgery. Across the seven major pharmaceutical markets a recent report estimated that around 37.6 million patients suffer from central neuropathic pain while some 170 million suffer from peripheral neuropathic pain (Neuropathic Pain Network and Pfizer Inc, 2006 survey).

Symptoms of neuropathic pain include a burning, shooting, stabbing or electric shock type sensations. Other common neuropathic pain symptoms are allodynia (pain due to normally non-painful stimuli), hyperesthesia (an exaggerated response to light touch) and hyperpathy (persistent pain even after the cause of the pain is removed) and dysthesia (abnormal and unpleasant tingling or pins and needles sensation).

Neuropathic pain is more common in certain patient populations. For example, up to a quarter of diabetic patients and a third of cancer patients experience such pain. Furthermore, over half of patients suffering from shingles develop post herpetic neuralgia and a third of patients with spinal injury are affected by neuropathic pain (Neuropathic Pain Network and Pfizer Inc, 2007 survey).

Currently, there are few effective treatments for neuropathic pain. Pregabalin, gabapentin, duloxetine (a serotonin-norepinephrine reuptake inhibitor (SNRI) anti-depressant), Δ9 tetrahydrocannibinol and lidocaine patches (for local treatment of post herpetic neuralgia) are amongst the currently available treatment options. Each, however, has its own distinct limitations. For example, pregabalin is associated with significant adverse CNS effects. The side effects most frequently leading to pregabalin discontinuation were dizziness and somnolence. These two side effects occurred in up to 30% of patients treated at the higher doses of pregabalin (FDA labeling). In the case of gabapentin, its oral bioavailability is not proportional to dose i.e., as dose is increased, bioavailability decreases. Bioavailabilities of approximately 60%, 47%, 34%, 33%, and 27% were observed following 900, 1200, 2400, 3600, and 4800 mg/day gabapentin (FDA labeling). Duloxetine is associated with nausea in 20-40% of treated patients, as well as suicidality concerns in treated patients (FDA labeling). Δ9 tetrahydrocannibinol has a distinct addiction liability (DEA classification).

Mexiletine, (rac)-1-(2,6-dimethylphenoxy)-2-propanamine hydrochloride (structure shown below) is a sodium channel blocking agent that has local anesthetic properties. Mexiletine first found utility as a Class 1B anti-arrhythmic agent, and is still used today to treat arrhythmias. The drug is currently available as 150 mg, 200 mg or 250 mg capsules administered TID and is currently licensed only for the treatment of ventricular arrhythmias. The most frequent adverse reaction associated with mexiletine administration is upper gastrointestinal distress i.e. nausea and vomiting (FDA label) and, in an attempt to miminse this, the drug is given in three divided doses each day even though its 12 h half life would allow less frequent dosing. The structure of mexiletine is shown below:—

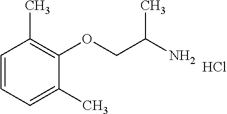

### Mexiletine (1-methyl-2-(2,6-xylyloxy)-ethylamine) hydrochloride

In recent years, mexiletine has found increasing utility in the treatment of muscle myotonias of different origin. One of the earliest studies was reported by Kwiecinski H et al (1992) Acta Neurol Scan 86, 371-375. In their comparative assessment of disopyamide, phenyloin, mexiletine and tocamide in some 30 patients with myotonic disorders, dramatic improvements were demonstrated with the latter two drugs. After either 1200 mg daily of tocamide (as three divided doses) or 600 mg daily of mexiletine (again as three divided doses) the reduction in the time taken for eye opening were 7 and 6-fold respectively, while hand opening time was reduced by 4.2 and 6.9-fold. Increase in the speed of stair step movement increased by 2.7 and 2.8 fold respectively. Although tocamide was therapeutically efficacious, its tendency to cause bone marrow suppression (Soff G A & Kadin M E (1987) Arch. Intern. Med. 147 598-599) precludes its acceptability in the long term use of myotonic conditions.

Although many patients with myotonia congentia can manage their disease without recourse to medication, for those patients needing drug therapy, Cannon S C et al (1996) (Trends Neurosci. 19 3-10), concluded that, “of the many drugs tested that can be administered orally, mexiletine is the drug of choice”.

Very recent work has confirmed the value of the use of mexiletine in treating muscle myotonias. A study reported by Logigian E L et al (2010) Neurol. 74, 1441-1448 in patients with myotonic dystrophy (type 1) showed that mexiletine treatment (150 tid or 200 mg tid) for periods of 7-weeks led to a 2-fold reduction in grip relaxation time.

Mexiletine has also been found to be of value in treating dystonia. Ohara S et al (1998) in Mov. Disord. 13, 934-40, reported on the use of mexiletine in the treatment of spasmodic torticollis. Torticollis, a condition in which the head is tilted to one side, is associated with muscle spasm, classically causing lateral flexion contracture of the cervical spine musculature. Spasmodic torticollis is also described as cervical dystonia. Ohara et al suggested that oral mexiletine therapy may be a safe and effective treatment for spasmodic torticollis. A later publication by Lucetti C et al (2000) Clin. Neuropharmacol. 23, 186-189) described the utility of mexiletine in the treatment of torticollis and generalised dystonia wherein these authors also concluded that mexiletine was a useful drug in the treatment of such conditions.

In more recent years, mexiletine has found increasing utility in the treatment of neuropathic pain of various origins. Its use has been reported for diabetic neuropathy, acute and chronic nerve pain, alcoholic polyneuropathy, chronic pain from radiotherapy, thalamic pain and diabetic truncal pain (Jarvis and Coukell (1998). Drugs 4, 691-707). Additionally more recent reports suggest the utility of mexiletine in the treatment of erythromelaglia (EM), a rare disabling disorder characterized by recurrent burning pain, erythema, and increased temperature of the affected areas (e.g., feet and ears). (Vivas A C et al (2010) Amer. J. Otolaryngology, May). Additionally mexiletine has been found to be useful in chronic cryptogenic sensory polyneuropathy, a condition in which patients present with numbness or tingling in the distal lower extremities (Wolfe G I et al (1999) Arch Neurol 56 540-547).

The use of mexiletine has however be associated with a relatively high incidence of nausea, vomiting and abdominal discomfort. In a study in the use of mexiletine in treating arrhthymias (Morganroth (1987). Am. J. Cardiol. 60, 1276-1281) showed up to 38% in patients encountered adverse GI events especially at higher doses. Such side effects are likely to contribute to poor patient compliance. Furthermore emesis may result in partial loss of the administered drug and consequently, a reduced and unpredictable efficacy. In extremis, vomiting can be a dose limiting side-effect of oral mexiletine and may preclude attainment of effective plasma drug concentrations. (Wright et al. (1997). Ann Pharmacother. 31, 29-34 and Galer et al. (1996). J Myotonic conditions Symptom Manage. 12, 161-167).

As to the mechanism of mexiletine's emetic action, currently there is only limited understanding. One experimental study has shown that mexiletine can decrease the slow-wave activity in the rat stomach in vivo, but had no effect on jejeunal myoelectrical activity (Bielefeldt and Bass (1991). Digestion 48, 43-50). Other in vitro work using the rabbit oesophageal sphincter suggested that mexiletine, like the intravenous anesthetic compounds ketamine and midazolam, may inhibit the non-adrenergic, non-cholinergic (NANC) relaxation brought about by nitric oxide (Kohjitani et al. (2003). Eur. J. Pharmacol., 465, 145-151). This study concluded that suppression of endogenous nitric oxide in the lower oesophageal sphincter smooth muscle by mexiletine may contribute to the adverse GI effects of mexiletine.

Studies conducted on another local anaesthetic agent lignocaine point to the emetic effects associated with oral administration of that compound being induced by a direct action on the gut. After equi effective iv and po anti-arrhythmic doses of the drug given to dogs only the orally administered drug induced emesis despite comparable systemic blood levels being reached in each. (Smith E R et al 1972) Amer. Heart Journal 83 363-372).

Mexiletine has been shown to inhibit gastric emptying in a dose dependent manner culiminating in gastric stasis at higher doses (Yoshkawa T et al (2002) Jpn Phamacol Ther 30, 979-984). Delayed gastric empyting or stasis is closely associated with nausea and vomiting and indeed antimetic drugs are invariably gastrokinetic agents. (Andrews P L et al 1988 Trends in Pharmacol Sci 9 334-331)

There are also reports in the literature which suggest that mexiletine may have inherent gastric irritant properties. For example periodic cases of oesphagistis following mexiletine ingestion have been reported (Penalba C (1986) Ann Gastroenterol Hepatol (Pris) 22, 267-268, Seggewiss R R & Seckfort H (1983) Dtsch Med. Wochenshr. 108 1018-1020, Addler J B (1990) Am J Gastroenterol. 85 629-630). Thus it is possible that the emetic effects of mexiletine could more simply be due to a direct irritant effect on the stomach.

In spite of such advances in understanding of the mechanism of these adverse events, there continues to be a need to reduce side-effects associated with mexiletine therapy. Although efficacy and toxicity are important considerations when administering any pharmaceutical compound, in the case of mexiletine the emetic properties are actually a greater barrier to patient compliance and to adequate and therapeutically effective dosing levels

There remains therefore a real need in the treatment of muscle myotonias for a mexiletine product which retains all the inherent pharmacological advantages of the drug molecule but overcomes its limitations in inducing adverse GI side-effects. The present invention addresses this need and the benefits provided by the compounds of the invention in reducing or eliminating emesis when treating with mexiletine are expected to be significant. The invention will thus provide easy access to treatment that was previously problematic for patients and clinicians.

### SUMMARY OF THE INVENTION

The present invention relates to prodrugs of mexilitine and mexilitine analogues and pharmaceutically acceptable salts of the same. The disclosure includes the use of such prodrugs in the treatment of muscle myotonias and dystonias. Advantageously, the prodrugs result in reduced or eliminated GI side effects such as emesis as compared to mexilitine.

According to one aspect, the present invention provides a prodrug of mexilitine or a mexilitine analogue or a pharmaceutically acceptable salt thereof for use in the treatment of muscle myotonias and dystonias, the prodrug having a structure of Formula I:

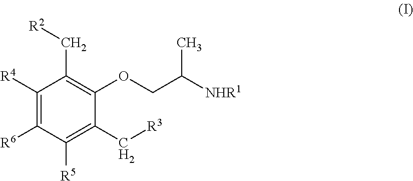

wherein

R¹ is selected from: H and a first prodrug-forming moiety selected from a group forming an amide or carbamate linkage directly to the remainder of the molecule;

each of R², R³, R⁴, R⁵ and R⁶ is independently selected from: H, OH and a second prodrug-forming moiety selected from a group forming an ester or carbamate linkage directly to the remainder of the molecule;

provided that the compound has a single prodrug moiety selected from the first and second prodrug moieties.

For all aspects and embodiments of the invention, those prodrugs which are a base or acid capable therefore of forming acid or base addition salts may be in the form of the free acid or free base compounds or in the form of a pharmaceutically acceptable acid addition salt or base addition salt thereof. The claims of this specification are therefore to be interpreted accordingly.

For example, R¹ may comprise a residue PRO¹ of a prodrug-forming moiety which, together with a carbonyl or oxy carbonyl group and the nitrogen of the adjoining NH, forms an amide or carbamate linkage between residue PRO¹ and the remainder of the molecule:

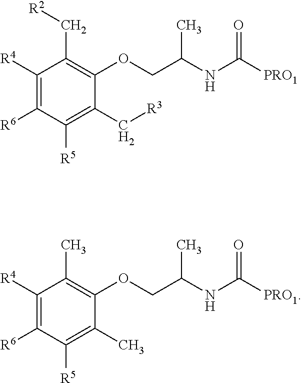

As another example, any one of R², R³, R⁴, R⁵ and R⁶ may comprise a residue PRO² of a prodrug-forming moiety which, together with a carbonyloxy or an aminocarbonyloxy group, forms an ester or carbamate linkage between residue PRO² and the remainder of the molecule, as illustrated below in the case of R⁶:

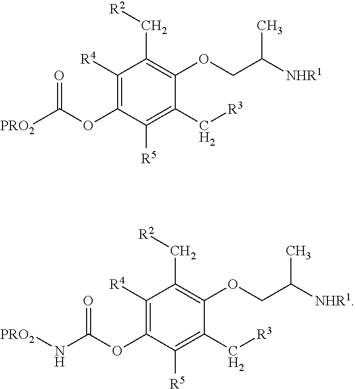

The same ester or carbamate structure may alternatively be formed at any one of R², R³, R⁴ and R⁵.

In an embodiment, the prodrug has a structure:

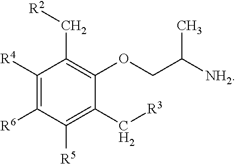

In an embodiment, the prodrug has a structure:

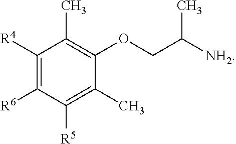

In an embodiment, the prodrug has a structure:

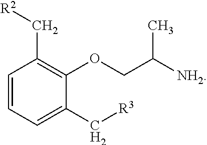

In an embodiment, the prodrug has a structure:

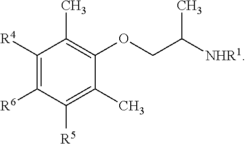

In an embodiment, the prodrug has a structure:

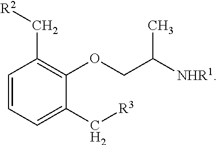

In an embodiment, the prodrug has a structure:

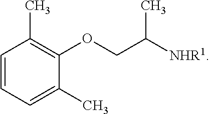

In an embodiment, prodrugging moieties of the prodrug, e.g. PRO₁ and PRO₂, are each an organic moiety i.e. comprising carbon and hydrogen and having up 10, 20, 30, 40 or 50 multivalent atoms and further comprising at least one heteroatom selected from O, S and N. Of course, in addition to a number of multivalent atoms, the prodrugging moieties will also include the required number of monovalent atoms, such as hydrogen atoms, which are covalently bonded to the multivalent atoms in order to satisfy the valency requirements of the multivalent atoms. Thus, for example, the prodrugging moiety glutamic acid,

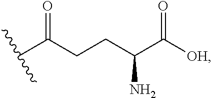

includes nine multivalent atoms and also includes eight hydrogen atoms.

In an embodiment, the prodrugging moieties of the prodrug have a molecular weight of less than 500 Daltons, and more preferably less than 300 Daltons. In a more preferred embodiment, the molecular weight of the prodrugging moiety is less than 200 Daltons.

In an embodiment, the prodrug has a structure of Formula II:

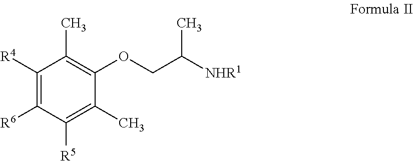

or a pharmaceutically acceptable salt thereof,
wherein,
R₁ is selected from the group consisting of: an amino acid, an amino acid residue terminating with a COOR^{g} group, an amino amide residue terminating with a CONR^{g}R^{h} group, an N-substituted amino acid, a peptide having 2 to 9 amino acids, a peptide having 2 to 9 amino acids and terminating with an amino acid residue terminating with a COOR^{g} group, a peptide having 2 to 8 amino acids and terminating with an amino amide residue terminating with a CONR^{g}R^{h} group, an N-substituted peptide having 2 to 9 amino acids and a moiety having the structure:

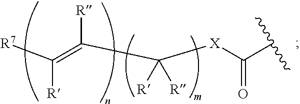

wherein,
m is 0, 1, 2, 3 or 4;
n is 0 or 1;
X is a bond or —O—;
R′ and R″ are each independently selected from the group consisting of: H, hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; and
R⁷ is selected from the group consisting of: H, substituted or unsubsititued aryl and substituted or unsubsititued heterocycle (e.g. substituted or unsubstituted heteroaryl) wherein the substituted aryl and substituted heterocycle (e.g. substituted heteroaryl) groups have 1, 2 or 3 substituents independently selected from the group consisting of: hydroxy, carboxy, oxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; and
R^{g} and R^{h} when present are each independently selected from the group consisting of: H, C₁₋₆ alkyl, —(CH₂)ₙ—C₃₋₆ cycloalkyl, phenyl and benzyl, or wherein R^{g} and R^{h} together with the nitrogen atom to which they are attached form a ring containing 3, 4, 5 or 6 carbon atoms; wherein each of the R^{g} and R^{h} groups may be unsubstituted or substituted with 1 or 2 substituent groups independently selected at each occurrence from the group consisting of: F, Cl, CN and OH;
s is an integer of 0 or 1;
R⁴, R⁵ and R⁶ are each independently selected from hydrogen and [symbol] CH.

In an embodiment, the prodrug has a structure of Formula II:

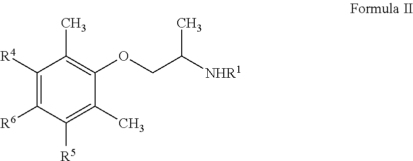

or a pharmaceutically acceptable salt thereof,
wherein,
R₁ is selected from the group consisting of: an amino acid, an N-substituted amino acid, a peptide having 2 to 9 amino acids, an N-substituted peptide having 2 to 9 amino acids and a moiety having the structure:

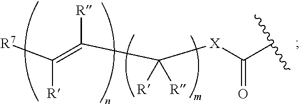

wherein,
m is 0, 1, 2, 3 or 4;
n is 0 or 1;
X is a bond or —O—;
R′ and R″ are each independently selected from the group consisting of: H, hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; and
R⁷ is selected from the group consisting of: H, substituted or unsubsititued aryl and substituted or unsubsititued heterocycle (e.g. substituted or unsubstituted heteroaryl) wherein the substituted aryl and substituted heterocycle (e.g. substituted heteroaryl) groups have 1, 2 or 3 substituents independently selected from the group consisting of: hydroxy, carboxy, oxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; and
R⁴, R⁵ and R⁶ are each independently selected from hydrogen and [symbol] CH.

In one embodiment, R⁴, R⁵ and R⁶ are each hydrogen.

In an embodiment, R^{g} is selected from the group consisting of: H, Me, Et and cyclopropyl. Preferably, R^{g} is H.

In an embodiment, R^{h} is selected from the group consisting of: H, Me, Et and cyclopropyl. Preferably, R^{h} is H.

In an embodiment, s is 0. In an embodiment, s is 1.

In an embodiment, n is 0. In this case, R⁷ is attached either directly to the methylene carbon to which R′ and R″ are bound, or (if m is also 0) R⁷ is bound directly to X.

In one embodiment, R¹ is an amino acid. In one embodiment, R⁴, R⁵ and R⁶ are each hydrogen and R¹ is an amino acid.

In one embodiment, R¹ is an amino amide residue terminating with a CONR^{g}R^{h} group. In one embodiment, R⁴, R⁵ and R⁶ are each hydrogen and R¹ is an amino amide residue terminating with a CONR^{g}R^{h} group.

In one embodiment, R¹ is an N-substituted amino acid. In one embodiment, R⁴, R⁵ and R⁶ are each hydrogen and R¹ is an N-substituted amino acid.

In one embodiment, R⁴, R⁵ and R⁶ are each hydrogen and R¹ is a peptide of 2 to 9 independently selected amino acids. In one embodiment, R⁴, R⁵ and R⁶ are each hydrogen and R¹ is a peptide of 2 to 3 independently selected amino acids. In one embodiment, R⁴, R⁵ and R⁶ are each hydrogen and R¹ is an N-substituted peptide of 2 to 9 independently selected amino acids. In one embodiment, R⁴, R⁵ and R⁶ are each hydrogen and R¹ is an N-substituted peptide of 2 to 3 independently selected amino acids.

In one embodiment, R⁴, R⁵ and R⁶ are each hydrogen and R¹ is a peptide having 2 to 8 amino acids and terminating with an amino amide residue terminating with a CONR^{g}R^{h} group. In one embodiment, R⁴, R⁵ and R⁶ are each hydrogen and R¹ is a peptide of 1 to 2 independently selected amino acids and terminating with an amino amide residue terminating with a CONR^{g}R^{h} group.

In one embodiment, R⁴, R⁵ and R⁶ are each hydrogen and R¹ is

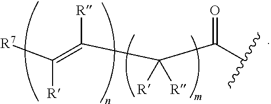

Optionally, R′ and R″ are each H.

In one embodiment, R⁴, R⁵ and R⁶ are each hydrogen, R¹ is

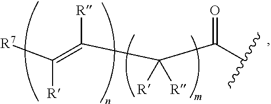

n is 0 and m is 0.

In one embodiment, R⁴, R⁵ and R⁶ are each hydrogen, R¹ is

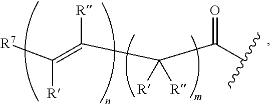

n is 0, m is 0 and R⁷ is substituted or unsubsititued aryl (e.g. substituted or unsubsititued phenyl) or substituted or unsubsititued heteroaryl (e.g. 3- or 4-pyridyl or 5-aminothiophen-2-carboxylic acid).

In one embodiment, R⁴, R⁵ and R⁶ are each hydrogen, R¹ is

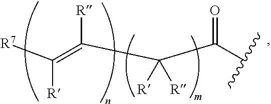

n is 1 and m is 0. Optionally, R′ and R″ are each H.

In one embodiment, R⁴, R⁵ and R⁶ are each hydrogen, R¹ is

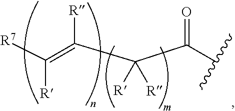

n is 0 and m is 1. Optionally, R′ and R″ are each H.

In one embodiment, R⁴, R⁵ and R⁶ are each hydrogen, R¹ is

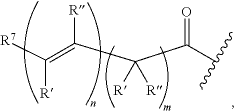

n is 0 and m is 2. Optionally, R′ and R″ are each H.

In one embodiment, R⁴, R⁵ and R⁶ are each hydrogen, R¹ is

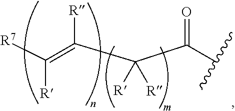

n is 1, m is 0 and R⁷ is substituted or unsubsititued heteroaryl (e.g. unsubsititued 5-imidazolyl). Optionally, R′ and R″ are each H.

In one embodiment, R⁴, R⁵ and R⁶ are each hydrogen, R¹ is

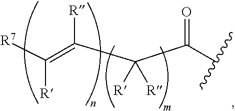

n is 0, m is 1 and R⁷ is substituted or unsubstituted heteroaryl (e.g. unsubsititued 3-indolyl). Optionally, R′ and R″ are each H.

In one embodiment, R⁴, R⁵ and R⁶ are each hydrogen, R¹ is

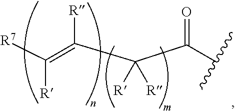

n is 0, m is 2 and R⁷ is substituted or unsubstituted heteroaryl (e.g. unsubsititued 5-imidazolyl). Optionally, R′ and R″ are each H.

In an embodiment, R⁷ is substituted aryl. In an embodiment, R⁷ is substituted phenyl, e.g. 4-hydroxy phenyl, 4-amino phenyl or 4-aminosalicylic acid. In an embodiment, R⁷ is substituted phenyl, e.g. phenyl substituted with carboxamido (—CONR^{g}R^{h}) or ester (—COOR^{g}).

In an embodiment, R⁷ is unsubsititued heteroaryl. In an embodiment, R⁷ is 3-pyridyl. In an embodiment, R⁷ is 4-pyridyl. In an embodiment, R⁷ is 5-aminothiophen-2-carboxylic acid. In an embodiment, R⁷ is unsubsititued 3-indolyl. In an embodiment, R⁷ is unsubsititued 5-imidazolyl.

In one embodiment, R⁴, R⁵ and R⁶ are each hydrogen, R¹ is

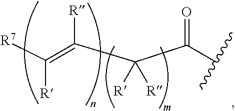

n is 0 and m is 4. Optionally, R′ and R″ are each H.

In one embodiment, R⁴, R⁵ and R⁶ are each hydrogen, R¹ is

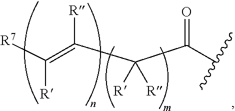

n is 0, m is 4 and R⁷ is substituted or unsubstituted heterocyclyl. Optionally, R′ and R″ are each H.

In an embodiment, R⁷ is 1,2-dithiolan-3-yl. In an embodiment, R³ is

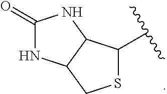

According to another aspect, the present invention provides a prodrug having a structure of Formula II:

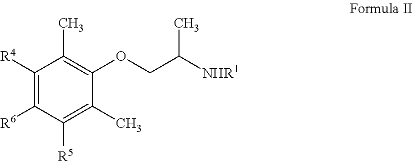

or a pharmaceutically acceptable salt thereof,
wherein,
R₁ is selected from the group consisting of: an amino amide residue terminating with a CONR^{g}R^{h} group, a peptide having 2 to 8 amino acids and terminating with an amino amide residue terminating with a CONR^{g}R^{h} group and a moiety having the structure:

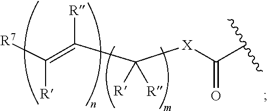

wherein,
m is 0, 1, 2, 3 or 4;
n is 0 or 1;
X is a bond or —O—;
R′ and R″ are each independently selected from the group consisting of: H, hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; and
R⁷ is selected from the group consisting of: substituted aryl and substituted heterocycle (e.g. substituted heteroaryl) wherein the substituted aryl and substituted heterocycle (e.g. substituted heteroaryl) groups have 1, 2 or 3 substituents independently selected from the group consisting of: COOR^{g}, provided that —COOR^{g} is not —COOH, and CONR^{g}R^{h};
R^{g} and R^{h} are each independently selected from the group consisting of: H, C₁₋₆ alkyl, —(CH₂)ₛ—C₃₋₆ cycloalkyl, phenyl and benzyl, or wherein R^{g} and R^{h} together with the nitrogen atom to which they are attached form a ring containing 3, 4, 5 or 6 carbon atoms; wherein each of the R^{g} and R^{h} groups may be unsubstituted or substituted with 1 or 2 substituent groups independently selected at each occurrence from the group consisting of: F, Cl, CN and OH;
s is an integer of 0 or 1;
R⁴, R⁵ and R⁶ are each independently selected from hydrogen and [symbol] CH.

In one embodiment, R⁴, R⁵ and R⁶ are each hydrogen.

In an embodiment, R^{g} is selected from the group consisting of: H, Me, Et and cyclopropyl. Preferably, R^{g} is H.

In an embodiment, R^{h} is selected from the group consisting of: H, Me, Et and cyclopropyl. Preferably, R^{h} is H.

In an embodiment, s is 0. In an embodiment, s is 1.

In an embodiment, n is 0. In this case, R⁷ is attached either directly to the methylene carbon to which R′ and R″ are bound, or (if m is also 0) R⁷ is bound directly to X.

In one embodiment, R¹ is an amino amide residue terminating with a COOR^{g} group. In one embodiment, R⁴, R⁵ and R⁶ are each hydrogen and R¹ is an amino amide residue terminating with a COOR^{g} group.

In one embodiment, R¹ is an amino amide residue terminating with a CONR^{g}R^{h} group. In one embodiment, R⁴, R⁵ and R⁶ are each hydrogen and R¹ is an amino amide residue terminating with a CONR^{g}R^{h} group.

In one embodiment, R⁴, R⁵ and R⁶ are each hydrogen and R¹ is a peptide having 2 to 8 amino acids and terminating with an amino amide residue terminating with a COOR^{g} group. In one embodiment, R⁴, R⁵ and R⁶ are each hydrogen and R¹ is a peptide of 1 to 2 independently selected amino acids and terminating with an amino amide residue terminating with a COOR^{g} group.

In one embodiment, R⁴, R⁵ and R⁶ are each hydrogen and R¹ is a peptide having 2 to 8 amino acids and terminating with an amino amide residue terminating with a CONR^{g}R^{h} group. In one embodiment, R⁴, R⁵ and R⁶ are each hydrogen and R¹ is a peptide of 1 to 2 independently selected amino acids and terminating with an amino amide residue terminating with a CONR^{g}R^{h} group.

In one embodiment, R⁴, R⁵ and R⁶ are each hydrogen and R¹ is

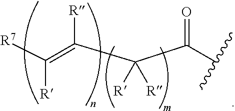

Optionally, R′ and R″ are each H.

In one embodiment, R⁴, R⁵ and R⁶ are each hydrogen, R¹ is

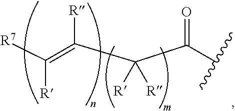

is 0 and m is 0.

In one embodiment, R⁴, R⁵ and R⁶ are each hydrogen, R¹ is

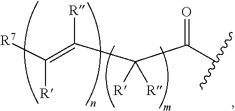

n is 0, m is 0 and R⁷ is substituted aryl (e.g. substituted phenyl) or substituted heteroaryl (e.g. substituted 3- or 4-pyridyl).

In one embodiment, R⁴, R⁵ and R⁶ are each hydrogen, R¹ is

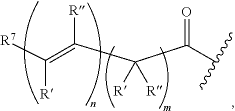

n is 1 and m is 0. Optionally, R′ and R″ are each H.

In one embodiment, R⁴, R⁵ and R⁶ are each hydrogen, R¹ is

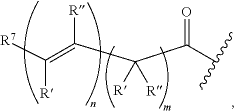

n is 0 and m is 1. Optionally, R′ and R″ are each H.

In one embodiment, R⁴, R⁵ and R⁶ are each hydrogen, R¹ is

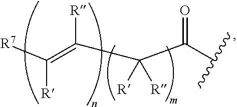

n is 0 and m is 2. Optionally, R′ and R″ are each H.

In one embodiment, R⁴, R⁵ and R⁶ are each hydrogen, R¹ is

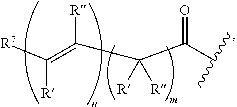

n is 1, m is O and R⁷ is substituted heteroaryl. Optionally, R′ and R″ are each H.

In one embodiment, R⁴, R⁵ and R⁶ are each hydrogen, R¹ is

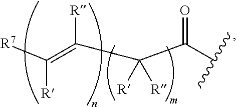

n is 0, m is 1 and R⁷ is substituted heteroaryl. Optionally, R′ and R″ are each H.

In one embodiment, R⁴, R⁵ and R⁶ are each hydrogen, R¹ is

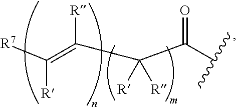

n is 0, m is 2 and R⁷ is substituted heteroaryl. Optionally, R′ and R″ are each H.

In an embodiment, R⁷ is substituted phenyl, e.g. phenyl substituted with carboxylate ester (—COOR^{g}).

In an embodiment, R⁷ is substituted phenyl, e.g. phenyl substituted with carboxamido (—CONR^{g}R^{h}).

In one embodiment, R⁴, R⁵ and R⁶ are each hydrogen, R¹ is

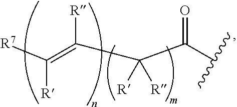

n is 0 and m is 4. Optionally, R′ and R″ are each H.

In one embodiment, R⁴, R⁵ and R⁶ are each hydrogen, R¹ is

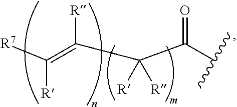

n is 0, m is 4 and R⁷ is substituted or unsubstituted heterocyclyl. Optionally, R′ and R″ are each H.

In an embodiment, the compounds of this aspect are for use as medicaments.

In an embodiment, the prodrug has a structure other than:

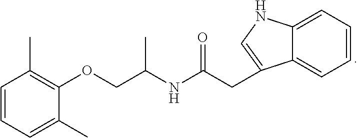

In an embodiment, the compounds of this aspect are for use in the treatment of muscle myotonia and dystonia.

In an embodiment, the compounds of this aspect are for use in the treatment of pain, e.g. neuropathic pain.

In the context of this invention, the term ‘amino acid’ includes moieties having a carboxylic acid group and an amino group. The term amino acid thus includes both natural amino acids (including proteinogenic amino acids) and non-natural amino acids. The term “natural amino acid” also includes other amino acids which can be incorporated into proteins during translation (including pyrrolysine and selenocysteine). Additionally, the term “natural amino acid” also includes other amino acids which are formed during intermediary metabolism e.g ornithine generated from arginine in the urea cycle. In one embodiment, the natural or non-natural amino acid may be optionally substituted with 1, 2 or 3 independently chosen substituents selected from halo and C₁₋₄ haloalkyl.

In one embodiment, the amino acid is selected from proteinogenic amino acids. Proteinogenic amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine and histidine.

In an embodiment, the amino acid is selected from the group consisting of: valine, leucine, isoleucine, aspartic acid, glutamic acid, serine, threonine, glutamine, arginine, lysine, proline, tyrosine, cysteine, methionine and histidine.

In an embodiment, the amino acid is selected from the group consisting of: valine, isoleucine, glutamic acid, serine, threonine, glutamine, arginine, lysine, proline, tyrosine, cysteine and histidine.

The term amino acid includes alpha amino acids and beta amino acids such as, but not limited to, beta alanine and 2-methyl beta alanine.

The term amino acid also includes certain lactam analogues of natural amino acids such as, but not limited to, pyroglutamine.

The term amino acid also includes amino acids homologues including homocitrulline, homoarginine, homoserine, homotyrosine, homoproline and homophenylalanine.

The terminal portion of the amino acid residue or peptide may be in the form of the free acid i.e. terminating in a —COOH group or may be in a masked (protected) form such as in the form of a carboxylate ester or carboxamide. Sometimes, the amino acid or peptide residue terminates with an amino group.

In an embodiment, the residue terminates with a carboxylic acid group —COOH or an amino group —NH₂. In another embodiment, the residue terminates with a carboxamide group CONR^{g}R^{h}. In an alternate embodiment, the residue terminates with a carboxylate ester COOR^{g}.

As mentioned above, the term “amino acid” includes compounds having a —COOH group and an —NH₂ group. A substituted amino acid includes an amino acid which has an amino group which is mono- or di-substituted. In particular, the amino group may be mono-substituted. (Of course, a proteinogenic amino acid may be substituted at another site from its amino group to form an amino acid which is a substituted proteinogenic amino acid). The term substituted amino acid thus includes N-substituted metabolites of the natural amino acids including, but not limited to, N-acetyl cysteine, N-acetyl serine, and N-acetyl threonine.

For example, the term “N-substituted amino acid” includes N-alkyl amino acids (e.g. C₁₋₆ N-alkyl amino acids such as sarcosine, N-methyl-alanine, N-methyl-glutamic acid and N-tert-butylglycine) which can include C₁₋₆ N-substituted alkyl amino acids (e.g. N-(carboxy alkyl) amino acids (e.g. N-(carboxymethyl)amino acids) and N-methylcycloalkyl amino acids (e.g. N-methylcyclopropyl amino acids)); N,N-di-alkyl amino acids (e.g. N,N-di-C₁₋₆ alkyl amino acids (e.g. N,N-dimethyl amino acid)); N,N,N-tri-alkyl amino acids (e.g. N,N,N-tri-C₁₋₆ alkyl amino acids (e.g. N,N,N-trimethyl amino acid)); N-acyl amino acids (e.g. C₁₋₆ N-acyl amino acid); N-aryl amino acids (e.g. N-phenyl amino acids, such as N-phenylglycine); N-amidinyl amino acids (e.g. an N-amidine amino acid, i.e. an amino acid in which an amine group is replaced by a guanidino group).

The term “amino acid” also includes amino acid alkyl esters (e.g. amino acid C₁₋₆ alkyl esters); and amino acid aryl esters (e.g. amino acid phenyl esters).

For amino acids having a hydroxy group present on the side chain, the term “amino acid” also includes O-alkyl amino acids (e.g. C₁₋₆ O-alkyl amino acid ethers); O-aryl amino acids (e.g. O-phenyl amino acid ethers); O-acyl amino acid esters; and O-carbamoyl amino acids.

For amino acids having a thiol group present on the side chain, the term “amino acid” also includes S-alkyl amino acids (e.g. C₁₋₆ S-alkyl amino acids such as S-methyl methionine, which can include C₁₋₆ S-substituted alkyl amino acids and S-methylcycloalkyl amino acids (e.g. S-methylcyclopropyl amino acids)); S-acyl amino acids (e.g. a C₁₋₆ S-acyl amino acid); S-aryl amino acid (e.g. a S-phenyl amino acid); a sulfoxide analogue of a sulfur-containing amino acid (e.g. methionine sulfoxide) or a sulfoxide analogue of an S-alkyl amino acid (e.g. S-methyl cystein sulfoxide) or an S-aryl amino acid.

In other words, the invention also envisages derivatives of natural amino acids such as those mentioned above which have been functionalized by simple synthetic transformations known in the art (e.g. as described in “Protective Groups in Organic Synthesis” by T W Greene and P G M Wuts, John Wiley & Sons Inc (1999), and references therein.

Examples of non-proteinogenic amino acids include, but are not limited to: citrulline, hydroxyproline, 4-hydroxyproline, β-hydroxyvaline, ornithine, β-amino alanine, albizziin, 4-amino-phenylalanine, biphenylalanine, 4-nitro-phenylalanine, 4-fluoro-phenylalanine, 2,3,4,5,6-pentafluoro-phenylalanine, norleucine, cyclohexylalanine, α-aminoisobutyric acid, α-aminobutyric acid, α-aminoisobutyric acid, 2-aminoisobutyric acid, 2-aminoindane-2-carboxylic acid, selenomethionine, lanthionine, dehydroalanine, γ-amino butyric acid, naphthylalanine, aminohexanoic acid, pipecolic acid, 2,3-diaminoproprionic acid, tetrahydroisoquinoline-3-carboxylic acid, tert-leucine, tert-butylalanine, cyclopropylglycine, cyclohexylglycine, 4-aminopiperidine-4-carboxylic acid, diethylglycine, dipropylglycine and derivatives thereof wherein the amine nitrogen has been mono- or di-alkylated.

Substituted amino groups include groups selected from:
(i) —NR^{c}R^{d}, optionally —NHR^{a};
(ii) —NR^{a}OH, optionally —NHOH;
(iii) —NR^{a}C(NR^{a})H, optionally —NHC(NH)H;
(iv) —NR^{a}C(NR^{a})NR^{a}OH, optionally —NHC(NH)NHOH;
(v) —NR^{a}C(NR^{a})NR^{a}CN, optionally —NHC(NH)NHCN;
(vi) —NR^{a}C(NR^{a})NR^{a}C(O)R^{a}, optionally —NHC(NH)NHC(O)R^{a};
(vii) —NR^{a}C(NR^{a})NR^{a}R^{b}, optionally —NHC(NH)NHR^{a};
(viii) —NR^{a}-G-C(O)OR^{a}, optionally —NH-G-C(O)OH;
(ix) —NR^{a}-G-C(O)R^{a}, optionally —NH-G-C(O)H;
wherein
G is a bond or is a linear or branched alkylene having 1, 2, 3, 4, 5 or 6 carbon atoms, especially linear alkylene, and is for example —CH₂—;

R^{a} and R^{b} are each independently an inert organic moiety, typically containing no more than 20 atoms which are not hydrogen or halogen; and
R^{c} and R^{d} are each independently hydrogen or a moiety in which the atoms other than hydrogen and halogen are selected from the group consisting of C, N, O and S and number from 1 to 20 (especially 1, 2, 3, 4, 5, 6 or 7) and which contains at least one hydrocarbyl group which is unsubstituted or substituted by halogen and may be aliphatic or carbocyclic, and is for example selected from aryl, alkyl, alkylene, cycloalkyl, cycloalkylene, alkenyl, alkenylene, cycloalkenyl, cycloalkenylene, alkynyl and alkynylene (which may be substituted by halogen and of which alkyl, alkylene, cycloalkyl and aryl form a preferred class), and optionally 1, 2 or 3 heteroatoms selected from O, N and S;
or R^{c} and R^{d} together with the attached nitrogen atom form optionally substituted heterocyclyl, for example imidazolyl, oxazolyl, thiazolyl, benzoxazolinyl or thiazolinyl.
In one embodiment, R^{a} and R^{b} are each independently hydrogen or a moiety in which the non-hydrogen atoms are selected from the group consisting of C, N, O and S and number from 1 to 20 (especially 1, 2, 3, 4, 5, 6 or 7, for example methyl, ethyl, butyl, propyl) and which contains at least one hydrocarbyl group which may be aliphatic or carbocyclic. Thus, for example, R^{a} and R^{b} may each be independently selected from aryl, alkyl, alkylene, cycloalkyl, cycloalkylene, alkenyl, alkenylene, cycloalkenyl, cycloalkenylene, alkynyl and alkynylene, and optionally 1, 2 or 3 heteroatoms selected from O, N and S.
In a further embodiment, R^{a} and R^{b} are each independently hydrogen or C₁₋₆ alkyl (especially C₁, C₂, C₃ or C₄ alkyl), carbocyclyl, —C₁₋₆ alkyl-carbocyclyl, -carbocyclyl-C₁₋₆ alkyl, or carbocyclyl (e.g. phenyl or cyclohexyl) optionally substituted by up to three moieties selected from C₁₋₆ alkyl, C₁₋₆ alkoxy and halogen. Those R^{a} and R^{b} groups which contain one or more alkylic carbon atoms may be interrupted at an alkylic carbon by an —O— linkage.
In a further embodiment, R^{a} and R^{b} are each independently selected from hydrogen, C₁₋₆ alkyl (e.g. methyl or ethyl), phenyl and cyclohexyl. Usually, at least one or both of R^{a} and R^{b} is hydrogen in groups containing —NR^{a}R^{b}.
In a further embodiment, R^{c} and R^{d} are each independently selected from hydrogen; C₁₋₆ alkyl optionally substituted with one or more substituents selected from hydrogen, halogen, carboxyl, C₁₋₆ alkoxy C₁₋₆ alkoxycarbonyl; carbocyclyl (especially phenyl or cyclohexyl) optionally substituted with one or more substituents selected from C₁, C₂, C₃ or C₄ alkyl; C₁, C₂, C₃ or C₄ alkoxy; and halogen; and -alkyl-carbocyclyl, wherein the carbocyclyl part is, for example, phenyl or cyclohexyl, and is optionally substituted with one or more substituents selected from C₁, C₂, C₃ or C₄ alkyl; C₁, C₂, C₃ or C₄ alkoxy; and halogen.
In a further embodiment, R^{c} and R^{d} are taken together with the attached nitrogen atom form optionally substituted heterocyclyl, for example imidazolyl, oxazolyl, thiazolyl, benzoxazolinyl or thiazolinyl, and of which is optionally substituted.

In an embodiment, the substituted amino group is —NR^{c}R^{d}, optionally —NHR^{a}.

In an embodiment, the substituted amino group is —NR^{a}C(NR^{a})NR^{a}R^{b}, optionally —NHC(NH)NHR^{a}.

In an embodiment, the substituted amino group is —NR^{a}-G-C(O)OR^{a}, optionally —NH-G-C(O)OH.

In an embodiment, the substituted amino group is —NR^{a}-G-C(O)R^{a}, optionally —NH-G-C(O)H.

Substituted amino groups include those substituted with a moiety which is joined to another atom in the molecule to form a 5- or 6-membered ring. The 5- or 6-membered ring may be saturated or wholly or partially unsaturated. The 5- or 6-membered ring may be unfused (monocyclic) or fused. Examples of such saturated monocyclic rings include piperidine and pyrrolidine. Examples of amino acids including such ring forming substituted amino groups include proline and pipecolic acid. Examples of such unsaturated monocyclic rings include pyridine, pyrimidine, pyrole and imidazole. The aforementioned saturated or unsaturated monocyclic rings may be fused to one or more rings e.g. to form indole, quinoline or quinazoline.

Substituted amino groups include mono-alkyl amino groups (e.g. C₁₋₆ mono-alkyl amino groups) which can include C₁₋₆ substituted alkyl amino groups (e.g. (carboxy alkyl)amino groups (e.g. (carboxymethyl)amino groups) and methylcycloalkyl amino groups (e.g. methylcyclopropyl amino groups)); di-alkyl amino groups (e.g. di-C₁₋₆ alkyl amino groups (e.g. dimethyl amino groups)); tri-alkyl amino groups (e.g. tri-C₁₋₆ alkyl amino groups (e.g. trimethyl amino groups)); acyl amino groups (e.g. C₁₋₆ acyl amino groups); aryl amino groups (e.g. phenyl amino groups); amidinyl amino acids (e.g. an amidine amino groups).

According to another aspect, the present invention is directed to novel compounds of the disclosure as such. The invention therefore includes compounds selected from the group consisting of: mexiletine-N-methylarginine amide, mexiletine-N,N-dimethylarginine amide, Mexiletine tryptophan amide, Mexiletine tyrosine amide, Mexiletine (indole-3-acetic acid) amide, Mexiletine-PHBA carbamate, Mexiletine [S-methyl-cysteine] amide, Mexiletine-PABA amide, Mexiletine (5-aminothiophene-2-carboxylic acid) amide, Mexiletine (4-aminosalicylic acid) amide, Mexiletine [O-carbamoyl-serine] amide, Mexiletine [N^{ε}-acetyl-lysine] amide, Mexiletine [methionine sulfoxide] amide, Mexiletine [N^{α}-acetyl-ornithine] amide, Mexiletine (urocanic acid) amide, Mexiletine dihydrourocanic acid amide, Mexiletine [S-methyl-cysteine sulfoxide] amide, Mexiletine [β-hydroxy-valine] amide, Mexiletine-glycocyamine amide, Mexiletine (carboxymethyl-glycine) amide, Mexiletine [N^{α}-acetyl-lysine] amide, Mexiletine [N^{ε}-acetyl-ornithine] amide, Mexiletine-aspartic acid amide, Mexiletine-Valine Amide, Mexiletine-Ornithine Amide, Mexiletine-valine-valine Amide, Mexiletine-Phenylalanine-Phenylalanine Amide, Mexiletine-albizziin amide, Mexiletine [trimethyl-lysine chloride] amide, Mexiletine-homoserine amide, Mexiletine-(4-Aminopiperidine-4-carboxylic acid) Amide, Mexiletine-[N,N′-dimethyl-lysine] amide, Mexiletine lipoic acid amide, Mexiletine biotin amide and Mexiletine ethyl carbamate amide. In an embodiment, the present invention includes compounds selected from the group consisting of: mexiletine-N-methylarginine amide, mexiletine-N,N-dimethylarginine amide, Mexiletine tyrosine amide, Mexiletine (indole-3-acetic acid) amide, Mexiletine-PHBA carbamate, Mexiletine [S-methyl-cysteine] amide, Mexiletine-PABA amide, Mexiletine (5-aminothiophene-2-carboxylic acid) amide, Mexiletine (4-aminosalicylic acid) amide, Mexiletine [O-carbamoyl-serine] amide, Mexiletine [N^{ε}-acetyl-lysine] amide, Mexiletine [methionine sulfoxide] amide, Mexiletine [N^{α}-acetyl-ornithine] amide, Mexiletine (urocanic acid) amide, Mexiletine dihydrourocanic acid amide, Mexiletine [S-methyl-cysteine sulfoxide]amide, Mexiletine [β-hydroxy-valine] amide, Mexiletine-glycocyamine amide, Mexiletine (carboxymethyl-glycine) amide, Mexiletine [N^{α}-acetyl-lysine] amide, Mexiletine [N^{ε}-acetyl-ornithine] amide, Mexiletine-Valine Amide, Mexiletine-Ornithine Amide, Mexiletine-valine-valine Amide, Mexiletine-Phenylalanine-Phenylalanine Amide, Mexiletine-albizziin amide, Mexiletine [trimethyl-lysine chloride] amide, Mexiletine-homoserine amide, Mexiletine-(4-Aminopiperidine-4-carboxylic acid) Amide, Mexiletine-[N,N′-dimethyl-lysine] amide, Mexiletine lipoic acid amide, Mexiletine biotin amide and Mexiletine ethyl carbamate amide. Chiral centres in the aforementioned molecules may be in the R or S configuration. The compounds may be for use as a medicament. The compounds may be for use in the treatment of myotonic conditions (e.g. neuropathic myotonic conditions) or dystonic conditions.

In one embodiment, the novel compounds are selected from the group consisting of: mexiletine-(S)—N-methylarginine amide, mexiletine-(S)—N,N-dimethylarginine amide, Mexiletine (S)-tryptophan amide, Mexiletine (S)-tyrosine amide, Mexiletine (indole-3-acetic acid) amide, Mexiletine-PHBA carbamate, Mexiletine [(R)—S-methyl-cysteine] amide, Mexiletine-PABA amide, Mexiletine (5-aminothiophene-2-carboxylic acid) amide, Mexiletine (4-aminosalicylic acid) amide, Mexiletine [O-carbamoyl-(S)-serine] amide, Mexiletine [(S)—N^{ε}-acetyl-lysine]amide, Mexiletine [(S)-methionine sulfoxide] amide, Mexiletine [N^{α}-acetyl-(S)-ornithine] amide, Mexiletine (urocanic acid) amide, Mexiletine dihydrourocanic acid amide, Mexiletine [(R)—S-methyl-cysteine sulfoxide] amide, Mexiletine [β-hydroxy-(S)-valine] amide, Mexiletine-glycocyamine amide, Mexiletine (carboxymethyl-glycine) amide, Mexiletine [(S)—N^{α}-acetyl-lysine] amide, Mexiletine [(S)—N^{ε}-acetyl-ornithine] amide, Mexiletine-(S)-aspartic acid amide, Mexiletine-(S)-Valine Amide, Mexiletine-(S)-Ornithine Amide, Mexiletine-valine-valine Amide, Mexiletine-(S)-Phenylalanine-(S)-Phenylalanine Amide, Mexiletine-(S)-albizziin amide, Mexiletine [trimethyl-(S)-lysine chloride] amide, Mexiletine-(S)-homoserine amide, Mexiletine-(4-Aminopiperidine-4-carboxylic acid) Amide, Mexiletine-[N,N′-dimethyl-(S)-lysine] amide, Mexiletine lipoic acid amide, Mexiletine biotin amide and Mexiletine ethyl carbamate amide.

In one embodiment, the novel compounds are selected from the group consisting of: mexiletine-(S)—N-methylarginine amide, mexiletine-(S)—N,N-dimethylarginine amide, Mexiletine (S)-tyrosine amide, Mexiletine (indole-3-acetic acid) amide, Mexiletine-PHBA carbamate, Mexiletine [(R)—S-methyl-cysteine] amide, Mexiletine-PABA amide, Mexiletine (5-aminothiophene-2-carboxylic acid) amide, Mexiletine (4-aminosalicylic acid) amide, Mexiletine [O-carbamoyl-(S)-serine] amide, Mexiletine [(S)—N^{α}-acetyl-lysine] amide, Mexiletine [(S)-methionine sulfoxide] amide, Mexiletine [N^{α}-acetyl-(S)-ornithine] amide, Mexiletine (urocanic acid) amide, Mexiletine dihydrourocanic acid amide, Mexiletine [(R)—S-methyl-cysteine sulfoxide] amide, Mexiletine [β-hydroxy-(S)-valine] amide, Mexiletine-glycocyamine amide, Mexiletine (carboxymethyl-glycine) amide, Mexiletine [(S)—N^{α}-acetyl-lysine] amide, Mexiletine [(S)—N^{ε}-acetyl-ornithine] amide, Mexiletine-(S)-Valine Amide, Mexiletine-(S)-Ornithine Amide, Mexiletine-valine-valine Amide, Mexiletine-(S)-Phenylalanine-(S)-Phenylalanine Amide, Mexiletine-(S)-albizziin amide, Mexiletine [trimethyl-(S)-lysine chloride] amide, Mexiletine-(S)-homoserine amide, Mexiletine-(4-Aminopiperidine-4-carboxylic acid) Amide, Mexiletine-[N,N′-dimethyl-(S)-lysine] amide, Mexiletine lipoic acid amide, Mexiletine biotin amide and Mexiletine ethyl carbamate amide.

According to another aspect, the present invention is directed to pharmaceutical compositions of the mexiletine prodrug. The compositions comprise at least one prodrug of the present invention, or pharmaceutically acceptable salt thereof, and at least one pharmaceutically acceptable excipient.

According to another aspect, the present invention is directed to a mexilitine prodrug for use in the treatment of muscle myotonias and dystonias, the prodrug having a structure according to Formula (III):

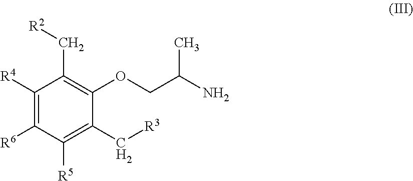

or a pharmaceutically acceptable salt thereof, wherein:

one of R², R³, R⁴, R⁵ and R⁶ is:

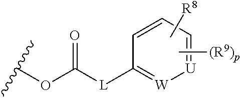

and the rest of R², R³, R⁴, R⁵ and R⁶ are each H;

L is a bond or is a linker moiety e.g. comprising a linear chain having a length of from 1 to 20 atoms (e.g. 1 to 10 atoms);
wherein R⁸ is selected from the group consisting of: —(CR′R″)ᵣCOOR^{g}, —(CR′R″)ᵣCONR^{g}R^{h},

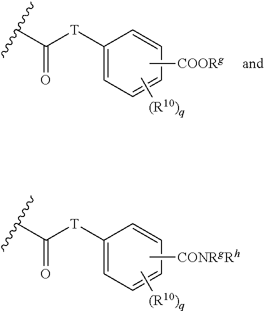

wherein T is —O— or —NR¹¹—; wherein R′ and R″ are each independently selected from the group consisting of: H, hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; wherein R^{g} and R^{h} when present are each independently selected from the group consisting of H, C₁₋₆ alkyl, —(CH₂)ₙ—C₃₋₆ cycloalkyl, phenyl and benzyl, or wherein R^{g} and R^{h} together with the nitrogen atom to which they are attached form a ring containing 3, 4, 5 or 6 carbon atoms; wherein each of the R^{g} and R^{h} groups may be unsubstituted or substituted with 1 or 2 substituent groups independently selected at each occurrence from the group consisting of: F, Cl, CN and OH; and wherein s is an integer of 0 or 1;

R¹¹ is selected from the group consisting of: H, C₁₋₄ alkyl (e.g. methyl, ethyl or propyl), C₁₋₄ haloalkyl (e.g. trifluoromethyl), alkoxy (e.g. methoxy, ethoxy or propoxy), C₁₋₄ haloalkoxy (e.g. trifluoromethoxy);

R⁹ and R¹⁰ are each independently selected from the group consisting of: hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;

W and U are each independently selected from the group consisting of: —CR′═ and —N═;

p is 0, 1 or 2;

q is 0, 1 or 2; and

r is 0, 1 or 2;

wherein each moiety R′ is independently selected from the others.

According to another aspect, the present invention is directed to a mexilitine prodrug for use in the treatment of muscle myotonias and dystonias, the prodrug having a structure according to Formula (III):

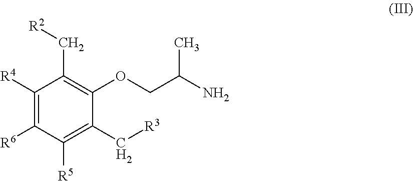

or a pharmaceutically acceptable salt thereof, wherein:

one of R², R³, R⁴, R⁵ and R⁶ is:

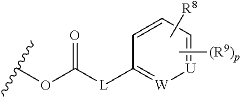

and the rest of R², R³, R⁴, R⁵ and R⁶ are each H;

L is a bond or is a linker moiety e.g. comprising a linear chain having a length of from 1 to 20 atoms (e.g. 1 to 10 atoms);

wherein R⁸ is selected from the group consisting of: —(CR′R″)ᵣCOOH and

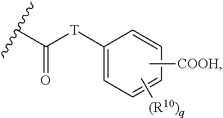

wherein T is —O— or —NR¹¹— and wherein R′ and R″ are each independently selected from the group consisting of: H, hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;

R¹¹ is selected from the group consisting of: H, C₁₋₄ alkyl (e.g. methyl, ethyl or propyl), C₁₋₄ haloalkyl (e.g. trifluoromethyl), C₁₋₄ alkoxy (e.g. methoxy, ethoxy or propoxy), C₁₋₄ haloalkoxy (e.g. trifluoromethoxy);

R⁹ and R¹⁰ are each independently selected from the group consisting of: hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;

W and U are each independently selected from the group consisting of: —CR′═ and —N═;

p is 0, 1 or 2;

q is 0, 1 or 2; and

r is 0, 1 or 2;

wherein each moiety R′ is independently selected from the others.

In an embodiment, L is —(CH₂)₁₋₆—, —NH— or a bond. In an embodiment, L is —NH—.

In an embodiment, R^{g} is selected from the group consisting of: H, Me, Et and cyclopropyl. Preferably, R^{g} is H.

In an embodiment, R^{h} is selected from the group consisting of: H, Me, Et and cyclopropyl. Preferably, R^{h} is H.

In an embodiment, s is 0. In an embodiment, s is 1.

In an embodiment, R⁴, R⁵ and R⁶ are each H and one of R² and R³ is

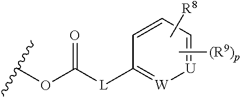

and the other of R² and R³ is H.

In an embodiment, R⁴, R⁵ and R⁶ are each H and one of R² and R³ is

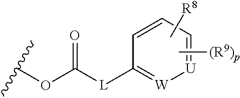

and the other of R² and R³ is H, L is a bond, W is ═C—, U is ═C—, p is 0 and R⁸ is —COOH.

In an embodiment, R⁴, R⁵ and R⁶ are each H and one of R² and R³ is

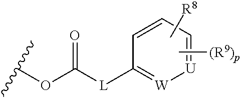

and the other of R² and R³ is H, L is a bond, W is ═C—, U is ═C—, p is 1, R⁸ is —COOH and R⁹ is OH.

In an embodiment, R⁴, R⁵ and R⁶ are each H and one of R² and R³ is

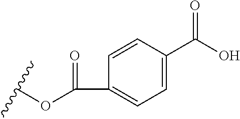

and the other of R² and R³ is H.

In an embodiment, R² and R³ are each H and one of R⁴, R⁵ and R⁶ is

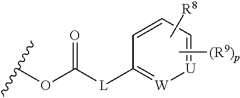

and the others of R⁴, R⁵ and R⁶ are H.

In an embodiment, R² and R³ are each H and one of R⁴, R⁵ and R⁶ is

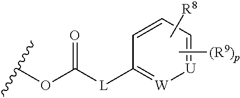

and the others of R⁴, R⁵ and R⁶ are H, L is —NH—, W is ═C—, U is ═C—, p is 0 and R⁸ is —COOH.

In an embodiment, R² and R³ are each H and one of R⁴, R⁵ and R⁶ is

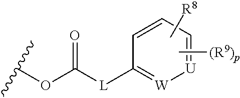

and the others of R⁴, R⁵ and R⁶ are H, L is —NH—, W is ═C—, U is ═C—, p is 1, R⁸ is —COOH and R⁹ is OH.

In an embodiment, R² and R³ are each H and one of R⁴, R⁵ and R⁶ is

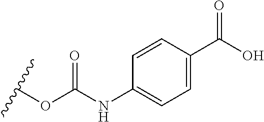

and the others of R⁴, R⁵ and R⁶ are H.

According to another aspect, the present invention is directed to a mexilitine prodrug the prodrug having a structure according to Formula (III):

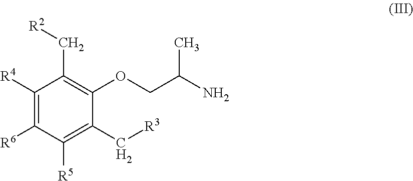

or a pharmaceutically acceptable salt thereof, wherein:

one of R², R³, R⁴, R⁵ and R⁶ is:

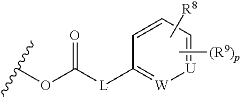

and the rest of R², R³, R⁴, R⁵ and R⁶ are each H;

L is a bond or is a linker moiety e.g. comprising a linear chain having a length of from 1 to 20 atoms (e.g. 1 to 10 atoms);
wherein R⁸ is selected from the group consisting of: —(CR′R″)ᵣCOOR^{g}, provided that —(CR′R″)ᵣCOOR^{g} is not —COOH, —(CR′R″)ᵣCONR^{g}R^{h},

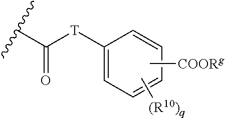

provided that when q is zero, —COOR^{g} is not —COOH, and

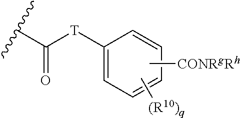

wherein T is —O—or —NR¹¹—; wherein R′ and R″ are each independently selected from the group consisting of: H, hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; wherein R^{g} and R^{h} when present are each independently selected from the group consisting of: H, C₁₋₆ alkyl, —(CH₂)ₛ—C₃₋₆ cycloalkyl, phenyl and benzyl, or wherein R^{g} and R^{h} together with the nitrogen atom to which they are attached form a ring containing 3, 4, 5 or 6 carbon atoms; wherein each of the R^{g} and R^{h} groups may be unsubstituted or substituted with 1 or 2 substituent groups independently selected at each occurrence from the group consisting of: F, Cl, CN and OH; and wherein s is an integer of 0 or 1;

R¹¹ is selected from the group consisting of H, C₁₋₄ alkyl (e.g. methyl, ethyl or propyl), C₁₋₄ haloalkyl (e.g. trifluoromethyl), C₁₋₄ alkoxy (e.g. methoxy, ethoxy or propoxy), C₁₋₄ haloalkoxy (e.g. trifluoromethoxy);

R⁹ and R¹⁰ are each independently selected from the group consisting of: hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;

W and U are each independently selected from the group consisting of: —CR′═ and —N═;

p is 0, 1 or 2;

q is 0, 1 or 2; and

r is 0, 1 or 2;

wherein each moiety R′ is independently selected from the others.

In an embodiment, L is —(CH₂)₁₋₆—, —NH— or a bond. In an embodiment, L is —NH—.

In an embodiment, R^{g} is selected from the group consisting of: H, Me, Et and cyclopropyl. Preferably, R^{g} is H.

In an embodiment, R^{h} is selected from the group consisting of: H, Me, Et and cyclopropyl. Preferably, R^{h} is H.

In an embodiment, s is 0. In an embodiment, s is 1.

In an embodiment, R⁴, R⁵ and R⁶ are each H and one of R² and R³ is

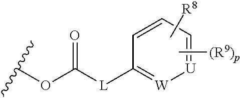

and the other of R² and R³ is H.

In an embodiment, R⁴, R⁵ and R⁶ are each H and one of R² and R³ is

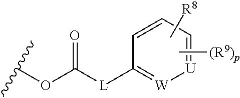

and the other of R² and R³ is H, L is a bond, W is ═C—, U is ═C— and p is 0.

In an embodiment, R⁴, R⁵ and R⁶ are each H and one of R² and R³ is

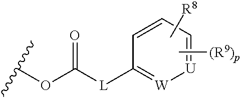

and the other of R² and R³ is H, L is a bond, W is ═C—, U is ═C—, p is 1 and R⁹ is OH.

In an embodiment, R² and R³ are each H and one of R⁴, R⁵ and R⁶ is

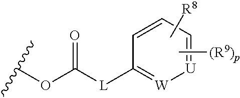

and the others of R⁴, R⁵ and R⁶ are H.

In an embodiment, R² and R³ are each H and one of R⁴, R⁵ and R⁶ is

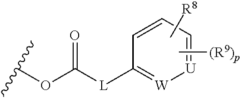

and the others of R⁴, R⁵ and R⁶ are H, L is —NH—, W is ═C—, U is ═C—, and p is 0.

In an embodiment, R² and R³ are each H and one of R⁴, R⁵ and R⁶ is

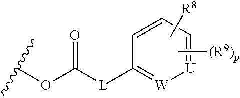

and the others of R⁴, R⁵ and R⁶ are H, L is —NH—, W is ═C—, U is ═C—, p is 1 and R⁹ is OH.

In an embodiment, the compounds of this aspect are for use as medicaments.

In an embodiment, the compounds of this aspect are for use in the treatment of muscle myotonias and dystonia.

In an embodiment, the compounds of this aspect are for use in the treatment of pain, e.g. neuropathic pain.

In another aspect, the present invention provides a method of treating a disorder in a subject in need thereof with mexiletine. The method comprises orally administering a mexiletine prodrug of the present invention, a pharmaceutically acceptable salt thereof, or composition thereof, to a subject or group of subjects in need thereof. The amount of the mexiletine prodrug is preferably a therapeutically effective amount. The disorder may be one treatable with mexiletine such as muscle myotonia or dystonia.

In an embodiment, a prodrug of the present invention confers the benefit of reduced adverse gastrointestinal side effects (such as nausea and vomiting), compared to the parent compound, while at the same time improving upon the rate and consistency of achievement of therapeutic plasma drug concentrations.

Accordingly, in one embodiment, the present invention is directed to a method for minimizing the gastrointestinal side effects normally associated with administration of mexiletine. The method comprises orally administering a mexiletine prodrug of the present invention, pharmaceutically acceptable salt thereof, or composition thereof, to a subject in need thereof, and wherein upon oral administration, the prodrug or pharmaceutically acceptable salt minimizes, if not completely avoids, the gastrointestinal side effects usually seen after oral administration of the unbound mexiletine. The amount of the mexiletine is preferably a therapeutically effective amount.

In a further embodiment, the GI side effect associated with administration of mexiletine is selected from, but is not limited to, emesis, nausea diahorrea and abdominal discomfort.

Another embodiment of the invention is directed to reducing the inter- and intra-subject variability of mexiletine serum levels. This will normally be during the treatment of myotonic conditions or dystonic conditions. The method comprises orally administering a mexiletine prodrug of the present invention, a pharmaceutically acceptable salt thereof, or composition thereof, to a subject or group of subjects in need thereof. The amount of the mexiletine prodrug is preferably a therapeutically effective amount.

Yet another embodiment of the invention related to improving the reproducibility of the bioavailability of mexiletine, in a subject in need thereof. The method comprises orally administering a mexiletine prodrug of the present invention, a pharmaceutically acceptable salt thereof, or composition thereof, to a subject or group of subjects in need thereof. The amount of the mexiletine prodrug is preferably a therapeutically effective amount.

Thus, in some embodiments, the present invention relates to natural and/or non-natural amino acids and short-chain peptide prodrugs of mexiletine or its prodrugged active metabolites. Without wishing to be bound to any particular theory, in an embodiment, the prodrug portion of the compound (i.e., the amino acid and/or peptide portion) serves to temporarily protect the gut from the local actions of the drug or its active metabolite (if administered in prodrugged form), while still delivering a pharmacologically effective amount of the drug/metabolite for the reduction or elimination of myotonic conditions. Such temporary inactivation should reduce the profound and highly undesirable emetic side-effects of this drug.

In an embodiment, the prodrugs of the present invention provide a means of sustaining plasma drug concentrations by the continuing generation of drug from prodrug—and also improving the reproducibility of bioavailability of the drug ensuring a more consistent patient response both within and between patients. These conferred attributes serve to ensure improved therapeutic efficacy and better patient compliance.

These and other embodiments of the invention are disclosed or are apparent from and encompassed by the following Detailed Description.

### BRIEF DESCRIPTION OF THE DRAWINGS

FIG. 1 is a graph showing the effects of (1) mexiletine, (2) mexiletine lysine amide, and (3) mexiletine glycine amide on electrical field stimulated contractions of isolated rabbit stomach circular smooth muscle preparation.

FIG. 2 is a graph showing the effects of mexiletine and mexiletine glutamic acid amide on 9-anthracene carboxylic acid (9-AC) induced myotonia in the rat (30 min post 9-AC ip injection) (i.e. the effects of mexiletine and mexiletine glutamic acid amide on time of righting reflex in rats displaying myonotia induced by 9 antrane carboxylic acid).

FIG. 3 is a graph showing the voltage protocol of the hNav1.x test procedure

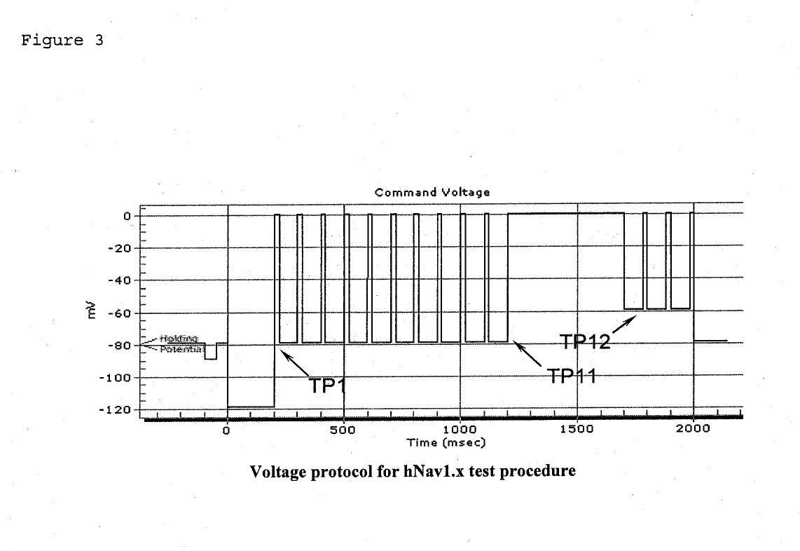

### DETAILED DESCRIPTION OF THE INVENTION

### Definitions

As used herein:

The term “peptide” refers to an amino acid chain consisting of 2 to 9 amino acids, unless otherwise specified. In preferred embodiments, the peptide used in the present invention is 2 or 3 amino acids in length. In one embodiment, a peptide can be a branched peptide. In this embodiment, at least one amino acid side chain in the peptide is bound to another amino acid (either through one of the termini or the side chain).

The term “N-substituted peptide” refers to an amino acid chain consisting of 2 to 9 amino acids in which one or more NH groups are substituted, e.g. by a substitutent described elsewhere herein in relation to substituted amino groups. Optionally, the N-substituted peptide has its N-terminal amino group substituted and, in one embodiment, the amide linkages are unsubstituted.

In one embodiment, an amino acid side chain is bound to another amino acid. In a further embodiment, side chain is bound to the amino acid via the amino acid's N-terminus, C-terminus, or side chain.

Examples of natural amino acid sidechains include hydrogen (glycine), methyl (alanine), isopropyl (valine), sec-butyl (isoleucine), —CH₂CH(CH₃)₂ (leucine), benzyl (phenylalanine), p-hydroxybenzyl (tyrosine), —CH₂OH (serine), —CH(OH)CH₃ (threonine), —CH₂-3-indoyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine). The natural amino acids are summarised in table 1:

TABLE 1
Natural Amino Acids (Used For Protein Biosynthesis)

| Amino acid | 3 letter code | 1-letter code |
|---|---|---|
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |

Suitably, natural amino acid sidechains may include isopropyl (valine), sec-butyl (isoleucine), p-hydroxybenzyl (tyrosine), —CH₂OH (serine), —CH(OH)CH₃ (threonine), —CH₂CH₂COOH (glutamic acid), —CH₂CH₂C(O)NH₂ (glutamine), —CH₂SH, (cysteine), —(CH₂)₄NH₂ (lysine), —(CH₂)₃NHC(═NH)NH₂ (arginine) and —CH₂-3-imidazoyl (histidine).

The term “amino” refers to a

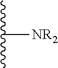

group, wherein each K is independently selected from the group consisting of: H and C₁-C₁₀ alkyl. For example, the term “amino” may refer to a

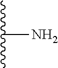

group.

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl, e.g. a C₁, C₂, C₃, C₄, C₅, C₆, C₇, C₈, C₉ or C₁₀ alkyl. For example, C₁₋₁₀ alkyl means a straight or branched saturated hydrocarbon chain containing, for example, at least 1, and at most 10, carbon atoms. Examples of “alkyl” groups, as used herein include, but are not limited to, methyl, ethyl, n-propyl, n-butyl, n-pentyl, isobutyl, isopropyl, t-butyl, hexyl, heptyl, octyl, nonyl and decyl.

The term “alkyl ester,” includes, for example, groups of the formulae

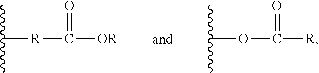

wherein each occurrence of R is independently a straight or branched C₁-C₁₀ alkyl group as defined immediately above.

The term “substituted alkyl” as used herein denotes alkyl radicals wherein at least one hydrogen is replaced by one more substituents such as, but not limited to, hydroxy, alkoxy (for example, C₁-C₁₀ alkoxy, e.g. methoxy or ethoxy), aryl (for example, phenyl), heterocycle, halogen (for example, F, Cl or Br), haloalkyl (for example, C₁-C₁₀ fluoroalkyl, e.g. trifluoromethyl or pentafluoroethyl), cyano, cyanomethyl, nitro, amino (e.g. a

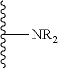

group, wherein each R is independently selected from the group consisting of: H and C₁-C₁₀ alkyl, or a

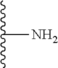

group), amide (e.g., —C(O)NH—R where R is a C₁-C₁₀ alkyl such as methyl), amidine (e.g., —C(═NR)NR₂, wherein each R is independently selected from the group consisting of: H and C₁-C₁₀ alkyl), amido (e.g., —NHC(O)—R where R is a C₁-C₁₀ alkyl such as methyl), carboxamide, carbamate (e.g. —NRC(O)OR, wherein each R is an independently selected C₁-C₁₀ alkyl, e.g. methyl), carbonate (e.g. —C(OR)₃ wherein each R is an independently selected C₁-C₁₀ alkyl, e.g. methyl), ester, alkoxyester (e.g., —C(O)O—R where R is a C₁-C₁₀ alkyl such as methyl) and acyloxyester (e.g., —OC(O)—R where R is a C₁-C₁₀ alkyl such as methyl). The definition pertains whether the term is applied to a substituent itself or to a substituent of a substituent.

The term “cycloalkyl” group as used herein refers to a non-aromatic monocyclic hydrocarbon ring of from 3 to 8 carbon atoms. Exemplary are saturated monocyclic hydrocarbon rings having 1, 2, 3, 4, 5, 6, 7 or 8, carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as those recited in the paragraph defining the substitutents of a “substituted alkyl”. The definition pertains whether the term is applied to a substituent itself or to a substituent of a substituent.

The term “heterocycle” refers to a stable 3- to 15-membered ring radical which consists of carbon atoms and from one to five heteroatoms selected from nitrogen, phosphorus, oxygen and sulphur. For example, a heterocyclic group may be:

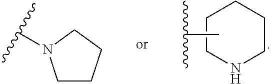

The term “substituted heterocycle” as used herein denotes a heterocycle group further bearing one or more substituents as set forth herein, such as those recited in the paragraph defining the substitutents of a “substituted alkyl”. The definition pertains whether the term is applied to a substituent itself or to a substituent of a substituent. For example, a substituted heterocyclic group may be:

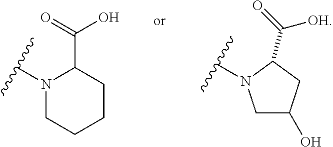

The term “aryl,” as used herein, refers to cyclic, aromatic hydrocarbon groups which have 1 to 3 aromatic rings, for example phenyl or naphthyl. The aryl group may have fused thereto a second or third ring which is a heterocyclo, cycloalkyl, or heteroaryl ring, provided in that case the point of attachment will be to the aryl portion of the ring system. Thus, exemplary aryl groups include

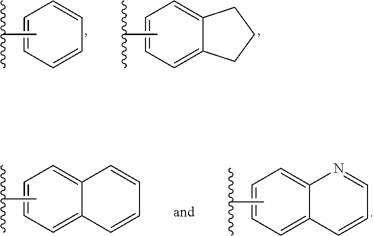

In embodiments, “aryl” refers to a ring structure consisting exclusively of hydrocarbyl groups.

The term “heteroaryl,” as used herein, refers to an aryl group in which at least one of the carbon atoms in the aromatic ring has been replaced by a heteroatom selected from oxygen, nitrogen and sulphur. The nitrogen and/or sulfur heteroatoms may optionally be oxidized and the nitrogen heteroatoms may optionally be quaternized. The heteroaryl group may be a 5 to 6 membered monocyclic, 7 to 11 membered bicyclic, or 10 to 16 membered tricyclic ring system. Thus, exemplary heteroaryl groups include

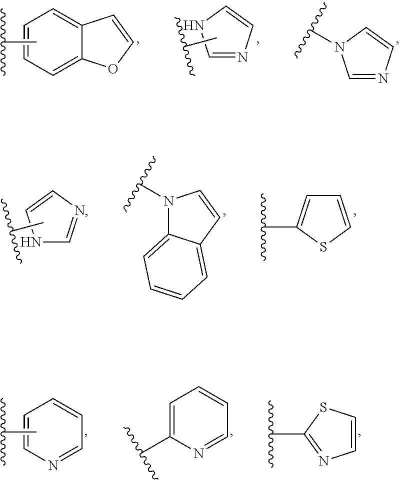

“Substituted aryl” and “substituted heteroaryl” groups refer to either an aryl or heteroaryl group, respectively, substituted by one or more substitutents at any point of attachment to the aryl or heteroaryl ring (and/or any further ring fused thereto). Exemplary substituents include hydroxy, carboxyl, alkoxy (for example, C₁-C₁₀ alkoxy, e.g. methoxy, ethoxy), aryl, phenyl, heterocycle, halogen (for example F, Cl, Br), haloalkyl (for example, C₁-C₁₀ haloalkyl, e.g. trifluoromethyl or pentafluoroethyl), cyano, cyanomethyl, nitro, amino (e.g. a

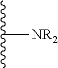

group, wherein each R is independently selected from the group consisting of: H and C₁-C₁₀ alkyl, or a

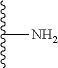

group), amide (e.g., —C(O)NH—R where R is a C₁-C₁₀ alkyl such as methyl), amidine (e.g., —C(═NR)NR₂, wherein each R is independently selected from the group consisting of: H and C₁-C₁₀ alkyl), amido (e.g., —NHC(O)—R where R is a C₁-C₁₀ alkyl such as methyl), carboxamide, carboxylic acid (e.g.,

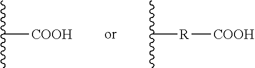

where R is a C₁-C₁₀ alkylene group such as —CH₂—), carbamate (e.g. —NRC(O)OR, wherein each R is an independently selected C₁-C₁₀ alkyl, e.g. methyl), carbonate (e.g. —C(OR)₃ wherein each R is an independently selected C₁-C₁₀ alkyl, e.g. methyl), ester, alkoxyester (e.g., —C(O)O—R where R is a C₁-C₁₀ alkyl such as methyl) and acyloxyester (e.g., —OC(O)—R where R is a C₁-C₁₀ alkyl such as methyl). For example, substituted aryl” and “substituted heteroaryl” groups include:

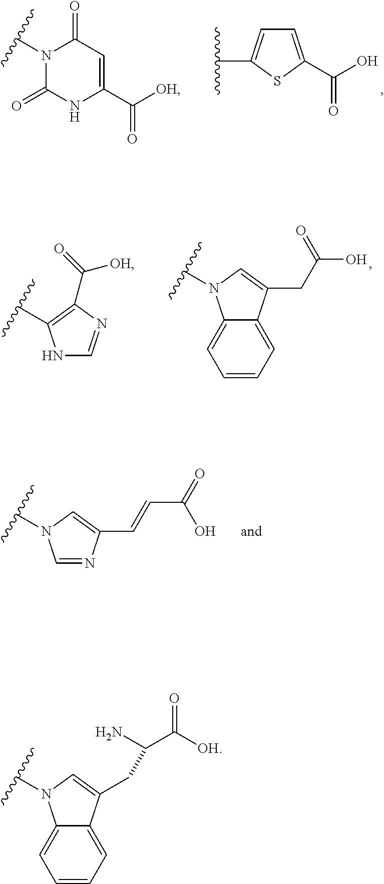

The terms “keto” and “oxo” are synonymous, and refer to the group ═O.

The term “acyl” includes moieties having the structure:

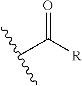

wherein R is C₁₋₆ alkyl or aryl.

“Amide,” as used herein, refers to the group

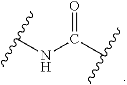

In the present invention, a prodrug moiety can be bonded to mexiletine via an amide linkage. In this embodiment, —N— is the amino nitrogen in the unbound mexiletine or mexiletine metabolite. An amide linkage can be formed by reacting an amine with a carboxylic acid. This is the reaction that forms a peptide bond.

The term “amino amide residue” refers to an amino acid fragment or residue that has been converted to an amide. The acid functionality in such a residue has been converted to an amide group so that the amino acid fragment contains both an amine group and an amide group.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “treating” includes: (1) preventing or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a patient to be treated is either statistically significant or at least perceptible to the patient or to the physician.

The term “subject” includes humans and other mammals, such as domestic animals (e.g., dogs and cats).

“Effective amount” means an amount of a prodrug or composition of the present invention sufficient to result in the desired therapeutic response. The therapeutic response can be any response that a user (e.g., a clinician) will recognize as an effective response to the therapy. The therapeutic response will generally be a reduction of myotonic or dystonic symptoms with minimisation of one or more of the gastrointestinal side effects that are present when mexiletine or hydroxylated mexiletine is administered in its underivatized form. It is further within the skill of one of ordinary skill in the art to determine appropriate treatment duration, appropriate doses, and any potential combination treatments, based upon an evaluation of therapeutic response.

The term “active ingredient,” unless specifically indicated, is to be understood as referring to mexiletine or a mexiletine metabolite portion of the prodrug, for example, the OH mexiletine portion of a prodrug of the present invention, as described herein.

“The term “salts” can include acid addition salts or addition salts of free bases. Suitable pharmaceutically acceptable salts (for example, of the carboxyl terminus of the amino acid or peptide) include, but are not limited to, metal salts such as sodium potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; and amino acid salts such as arginate, gluconate, galacturonate, alaninate, asparginate and glutamate salts (see, for example, Berge, et al. (1977). “Pharmaceutical Salts,” J. Pharma. Sci. 66, 1).

The term “bioavailability,” as used herein, generally means the rate and/or extent to which the mexiletine or a hydroxylated mexiletine is absorbed from a drug product and becomes systemically available, and hence available at the site of action. See Code of Federal Regulations, Title 21, Part 320.1 (2003 ed.). For oral dosage forms, bioavailability relates to the processes by which the active ingredient is released from the oral dosage form and moves to the site of action. Bioavailability data for a particular formulation provides an estimate of the fraction of the administered dose that is absorbed into the systemic circulation. Thus, the term “oral bioavailability” refers to the fraction of a dose of mexiletine given orally that is absorbed into the systemic circulation after a single administration to a subject. A preferred method for determining the oral bioavailability is by dividing the AUC of the mexiletine given orally by the AUC of the same mexiletine dose given intravenously to the same subject, and expressing the ratio as a percent. Other methods for calculating oral bioavailability will be familiar to those skilled in the art, and are described in greater detail in Shargel and Yu, Applied Biopharmaceutics and Pharmacokinetics, 4th Edition, 1999, Appleton & Lange, Stamford, Conn., incorporated herein by reference in its entirety.

### ADVANTAGES OF THE COMPOUNDS OF THE INVENTION

Any locally mediated emesis (i.e., from within the gut lumen) associated with the administration of mexiletine can be potentially reduced if mexiletine could be transiently inactivated until absorbed. This inactivation can preclude direct exposure of the drug to the lower oesophageal sphincter and stomach. An inactive prodrug of mexiletine that is only activated post absorption could be one way of reducing or eliminating emesis and other adverse GI effects. As an alternative approach, prodrugs of active mexiletine metabolites can be employed (e.g., p-OH, m-OH mexiletine or hydroxymethylmexiletine). Para-hydroxymexiletine has been reported to retain around 25% of the sodium channel inhibitory activity of the parent molecule and may therefore be a useful drug in its own right (De Bellis et al. (2006). Brit. J. Pharmacol. 149, 300-310). If the adverse GI side effects (e.g., emesis) associated with mexiletine or its active metabolite could be satisfactorily overcome by transient inactivation, the resultant product could provide a much improved treatment for muscle myotonias and dystonia.

Without wishing to be bound to any particular theory, it is believed that the amino acid or peptide portion of the mexiletine or mexiletine metabolite prodrugs provided herein are able to selectively exploit the inherent di- and tripeptide transporter Pept1 within the digestive tract to effect absorption. Once absorbed, the prodrugs are subjected to hydrolysis, releasing the active drug into the systemic circulation. It is believed that mexiletine is subsequently released from the amino acid or peptide prodrug by hepatic and extrahepatic hydrolases that are, in part, present in blood and or plasma.

Such assisted absorption of the prodrugs by Pept1 may provide greater consistency in response possibly as the result of more consistent, oral bioavailability. As a result of this more reproducible oral bioavailability, the prodrugs of the present invention offer a significant reduction of inter- and intra-subject variability of mexiletine plasma and CNS concentrations and, hence, significantly less fluctuation in the alleviation of myotonic and dystonic symptoms. Thus, patient compliance is likely to be further improved as the result of this greater predictability of therapeutic response.

Additionally, single amino acids and peptides particularly those natural amino acids or those generated during intermediary metabolism would not be expected to present a toxicity risk. The amino acid or peptide would advantageously be expected to transiently inactivate mexiletine or its active metabolites due the profound change in overall structure and conferred water solubility. Additionally, judiciously chosen peptide conjugates could offer the potential for protracted or sustained release by their partial hydrolysis by peptidases such as trypsin, within the gut lumen. For example, the introduction of a C-terminus poly-arginine or poly-lysine fragment to the drug either directly or indirectly (e.g., through another amino acid such as glycine) may result in partial hydrolysis in the gut lumen and hence control the rate of delivery of the resultant potentially absorbable di- or tripeptidomimetic compound for absorption. Such absorption is then likely to be effected by active transporters such as Pept1, which is specific for di- and tripeptides.

Peptides comprising any of the naturally occurring amino acids, as well as non-natural amino acids and those resulting from intermediary metabolism, can be used in the prodrugs of the present invention. If non-natural amino acids are employed as a peptide prodrug moiety, or portion thereof, the peptide can include solely non-natural amino acids, or alternatively, a combination of natural and non-natural amino acids.

The amino acids employed in the prodrugs for use with the present invention are preferably in the L configuration. The present invention also contemplates prodrugs of the invention comprised of amino acids in the D configuration, or mixtures of amino acids in the D and L configurations.

Representative Amino Acids and Peptides for Use with the Present Invention

Preferred mexiletine prodrugs of the present invention include: mexilitine glutamic acid amide, mexiletine aspartic acid amide, mexiletine S-methyl-methionine chloride amide, mexiletine [(S)—N^{α}-acetyl-lysine] amide, mexiletine[(R)—S-methylcysteine sulphoxide amide, mexiletine homoarginine amide, mexiletine (carboxymethyl-glycine) amide, mexiletine-glycocyamine amide, mexiletine (S)—N-methylarginine amide and mexiletine (S)—N,N-dimethylarginine amide.

Without wishing to be bound to any particular theory, it is believed that the amino acid or peptide portion of the mexiletine or p-OH, m-OH or hydroxymethyl mexiletine prodrug selectively exploits the inherent di- and tripeptide transporter Pept1 within the digestive tract. Once absorbed, it is thought that the prodrugs will be subject to hydrolysis, releasing the active drug into the systemic circulation. Avoidance of direct contact between active drug and gut wall reduces and/or minimizes the risk of emesis while the assisted absorption of the prodrug by Pept1 ensures more consistent plasma drug levels.

### Salts, Solvates, Stereoisomers, Derivatives of the Compounds of the Invention

The representative salts described below are directed to mexiletine and prodrugs of mexiletine metabolites (e.g., p-OH, m-OH mexiletine or hydroxymethylmexiletine).

The methods of the present invention further encompass the use of salts, solvates, stereoisomers of the prodrugs of mexiletine/mexiletine metabolites described herein. In one embodiment, the invention disclosed herein encompasses all pharmaceutically acceptable salts of mexiletine prodrugs.

Typically, a pharmaceutically acceptable salt of a prodrug of mexiletine used in the practice of the present invention is prepared by reaction of the prodrug with a desired acid as appropriate. In the case of the p-OH mexiletine metabolite prodrug this could alternatively involve making a salt of the free carboxylic function. The salt may precipitate from solution and be collected by filtration or may be recovered by evaporation of the solvent. For example, an aqueous solution of an acid such as hydrochloric acid may be added to an aqueous suspension of the prodrug and the resulting mixture evaporated to dryness (lyophilized) to obtain the acid addition salt as a solid. Alternatively, the prodrug may be dissolved in a suitable solvent, for example, an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the prodrugs may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts of prodrugs of the p-OH mexiletine metabolite are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine and N-methylglucamine.

The base addition salts of the acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention of the p-OH mexiletine metabolite may have both a basic and an acidic center and may therefore be in the form of zwitterions.

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, e.g., Brittain, Polymorphism in Pharmaceutical solids. Marcel Decker, New York, 1999). The compounds useful in the practice of the present invention can have one or more chiral centers and, depending on the nature of individual substituents, they can also have geometrical isomers.

Individual isomers of the mexiletine (or mexiletine metabolite) prodrugs described herein may be used to practice the present invention. The description or naming of a particular compound in the specification and claims is intended to include both individual enantiomers of the prodrug, as well as mixtures of enantiomers (racemic or otherwise) of the prodrug. Methods for the determination of stereochemistry and the resolution of stereoisomers are well-known in the art.

The invention thus encompasses any tautomeric forms of the compounds of Formula (I) as well as geometrical and optical isomers. Thus, it is contemplated that the present invention specifically includes tautomers of Formula (I) or pharmaceutical salts thereof.

### Pharmaceutical Compositions of the Invention

While it is possible that, for use in the methods of the invention, the prodrug of the present invention may be administered as the isolated substance, the active ingredient may be presented in a pharmaceutical composition, e.g., wherein the agent is in admixture with a pharmaceutically acceptable carrier selected with regard to the intended route of administration and standard pharmaceutical practice. In one embodiment of the present invention, a composition comprising an mexilitine prodrug of the present invention (e.g., a prodrug of any of the Formulae provided). The composition comprises at least one mexilitine prodrug selected from the Formula provided, and at least one pharmaceutically acceptable excipient or carrier.

The formulations of the invention may be immediate-release dosage forms, i.e., dosage forms that release the prodrug at the site of absorption immediately, or controlled-release dosage forms, i.e., dosage forms that release the prodrug over a predetermined period of time. Controlled release dosage forms may be of any conventional type, e.g., in the form of reservoir or matrix-type diffusion-controlled dosage forms; matrix, encapsulated or enteric-coated dissolution-controlled dosage forms; or osmotic dosage forms. Dosage forms of such types are disclosed, e.g., in Remington, The Science and Practice of Pharmacy, 20^{th} Edition, 2000, pp. 858-914. The formulations of the present invention can be administered from one to six times daily, depending on the dosage form and dosage.

However, since absorption of amino acid and peptide prodrugs of mexiletine/p-OH mexiletine metabolite may proceed via active transporters located in specific regions of GI tract, unconventional controlled dosage forms may be desirable. For example, the Pept1 transporter is believed to be largely confined to the upper GI tract, and should it be a contributor to prodrug absorption, may limit the effectiveness for continued absorption along the whole length of the GI tract.

For those prodrugs of mexiletine/hydroxylated mexiletine which do not result in sustained plasma drugs levels due to continuous generation of active agent from a plasma reservoir of prodrug—but which may offer other advantages—gastroretentive or mucoretentive formulations analogous to those used in metformin products such as Glumetz® or Gluphage XR® may be useful. The former exploits a drug delivery system known as Gelshield Diffusion™ Technology while the latter uses a so-called Acuform™ delivery system. In both cases the concept is to retain drug in the stomach, slowing drug passage into the ileum maximizing the period over which absorption take place and effectively prolonging plasma drug levels. Other drug delivery systems affording delayed progression along the GI tract, such as mucoadhesive formulations, may also be of value.

For those mexiletine prodrugs that do not require the sophistication of the aforementioned delivery systems conventional formulations as described below should be adequate.

In one embodiment, the present invention provides a pharmaceutical composition comprising at least one active pharmaceutical ingredient (i.e., a prodrug of mexiletine or hydroxy mexiletine), or a pharmaceutically acceptable derivative (e.g., a salt or solvate) thereof, and a pharmaceutically acceptable carrier or excipient. In particular, the invention provides a pharmaceutical composition comprising a therapeutically effective amount of at least one prodrug of the present invention, or a pharmaceutically acceptable derivative thereof, and a pharmaceutically acceptable carrier or excipient.

The prodrug employed in the present invention may be used in combination with other therapies and/or active agents. Accordingly, the present invention provides, in another embodiment, a pharmaceutical composition comprising at least one compound useful in the practice of the present invention, or a pharmaceutically acceptable salt or solvate thereof, a second active agent, and, optionally a pharmaceutically acceptable carrier or excipient.

When combined in the same formulation, it will be appreciated that the two compounds are preferably stable and compatible with each other and the other components of the formulation. When formulated separately, they may be provided in any convenient formulation, conveniently in such manner as are known for such compounds in the art. In some embodiments, the two compounds are either (1) two distinct prodrugs of mexiletine, (2) a prodrug of mexiletine and a prodrug of p-OH mexiletine, (3) two prodrugs of p-OH mexiletine, (4) a prodrug of mexiletine and a prodrug of m-OH mexiletine, (5) a prodrug of mexiletine and a prodrug of hydroxymethylmexiletine, (6) two prodrugs of m-OH mexiletine, (7) two prodrugs of hydroxymethylmexiletine, (8) a prodrug of meta-OH mexiletine and a prodrug of p-OH mexiletine or (9) a prodrug of hydroxymethylmexiletine and a prodrug of p-OH mexiletine. In other embodiments, the two compounds include a prodrug of Formula I and another compound for a distinct indication.

The prodrugs presented herein may be formulated for administration in any convenient way for use in human or veterinary medicine. The invention therefore includes pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may also be used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, see, e.g., International Patent Application No. WO 02/00196 (SmithKline Beecham).

The compounds and pharmaceutical compositions of the present invention are intended to be administered orally (e.g., as a tablet, sachet, capsule, pastille, pill, bolus, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia, cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium aluminium silicate, polyethylene glycol or bentonite.

Examples of pharmaceutically acceptable fillers for oral compositions useful herein include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers useful herein include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants useful herein include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

The pharmaceutical compositions of the invention may contain from 0.01 to 99% weight per volume of the prodrugs encompassed by the present invention.

### Dosages

The doses referred to throughout the specification refer to the amount of mexiletine free base equivalents, unless otherwise specified.

Appropriate patients to be treated according to the methods of the invention include any human or animal in need of treatment. Methods for the diagnosis and clinical evaluation of the myotonic and/or dystonic conditions, including the severity of that condition experienced by an animal or human are well known in the art. Thus, it is within the skill of the ordinary practitioner in the art (e.g., a medical doctor or veterinarian) to determine if a patient is in need of treatment. The patient is preferably a mammal, more preferably a human, but can be any animal, including a laboratory animal in the context of a clinical trial, screening, or activity experiment employing an animal model. Thus, as can be readily appreciated by one of ordinary skill in the art, the methods and compositions of the present invention are particularly suited to administration to any animal or subject, particularly a mammal, and including, but not limited to, domestic animals, such as feline or canine subjects, farm animals, such as but not limited to bovine, equine, caprine, ovine, and porcine subjects, research animals, such as mice, rats, rabbits, goats, sheep, pigs, dogs, cats, etc., avian species, such as chickens, turkeys, songbirds, etc.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Mexitil®, the FDA approved mexiletine hydrochloride formulation, is available in 150 mg, 200 mg and 250 mg capsules. 100 mg of mexiletine hydrochloride is equivalent to 83.31 mg of mexiletine base. Typically, Mexitil® is administered every 8 hours. In one embodiment of the invention, the prodrug dose is selected from one of the doses of Mexitil®, and can be administered once every eight hours. In another embodiment, the prodrug dose is selected from one of the doses of Mexitil®, and can be administered once every twelve or twenty four hours

In one embodiment, an effective daily dose of the mexiletine prodrug is from 1 mg to 2000 mg, preferably from 100 mg to 2000 mg, of the prodrug. For example, the prodrugs encompassed by the present invention may be formulated in a dosage form that provides from about 200 mg to about 2000 mg of the prodrug per day, preferably from about 200 mg to about 1000 mg of the prodrug per day. In a preferred embodiment, an effective amount of the a prodrug of the present invention is either 250 mg, 500 mg, 750 mg, /day.

In another embodiment, an effective daily dose of the p-OH mexiletine prodrug is from 4 mg to 8000 mg, preferably from 400 mg to 8000 mg, of the prodrug. In an alternative embodiment, an effective daily amount of the p-OH mexiletine prodrug is either 1000 mg or 3000 mg.

Depending on the severity of the myotonic or dystonic condition to be treated, a suitable therapeutically effective and safe dosage, as may readily be determined within the skill of the art, can be administered to subjects. For oral administration to humans, the daily dosage level of the prodrug may be in single or divided doses. The duration of treatment may be determined by one of ordinary skill in the art, and should reflect the nature of the myotonic conditions (e.g., a chronic versus an acute condition) and/or the rate and degree of therapeutic response to the treatment. Typically, a physician will determine the actual dosage which will be most suitable for an individual subject.

In the methods of treating muscle myontonias, the prodrugs encompassed by the present invention may be administered in conjunction with other therapies and/or in combination with other active agents. For example, the prodrugs encompassed by the present invention may be administered to a patient in combination with other active agents used in the management of the condition. An active agent to be administered in combination with the prodrugs encompassed by the present invention may include, for example, a drug selected from the group consisting of quinine, procainamide, tocamide, phenyloin and acetazolamide. In such combination therapies the prodrugs encompassed by the present invention may be administered prior to, concurrent with, or subsequent to the other therapy and/or active agent.

Where the prodrugs encompassed by the present invention are administered in conjunction with another active agent, the individual components of such combinations may be administered either sequentially or simultaneously in separate or combined pharmaceutical formulations by any convenient route. When administration is sequential, either the prodrugs encompassed by the present invention or the second active agent may be administered first. For example, in the case of a combination therapy with another active agent, the prodrugs encompassed by the present invention may be administered in a sequential manner in a regimen that will provide beneficial effects of the drug combination. When administration is simultaneous, the combination may be administered either in the same or different pharmaceutical composition. For example, a prodrug encompassed by the present invention and another active agent may be administered in a substantially simultaneous manner, such as in a single capsule or tablet having a fixed ratio of these agents, or in multiple separate dosage forms for each agent.

When the prodrugs of the present invention are used in combination with another agent active in the methods for treating myotonic conditions, the dose of each compound may differ from that when the compound is used alone. Appropriate doses will be readily appreciated by those of ordinary skill in the art.

### Methods of the Invention

One embodiment of the present invention is a method of treating a disorder in a subject in need thereof with mexiletine. The method comprises orally administering a mexiletine prodrug of the present invention, pharmaceutically acceptable salt thereof, or composition thereof, to a subject in need thereof. The amount of the mexiletine is preferably a therapeutically effective amount. The disorder may be one treatable with mexiletine. For example, the disorder may be neuropathic myotonic or dystonic conditions.

In a further embodiment of the invention, a method is provided for treating a disorder in a subject in need thereof with mexiletine, without inducing GI side effects associated with mexiletine. The method comprises orally administering a mexiletine prodrug of the present invention, pharmaceutically acceptable salt thereof, or composition thereof, to a subject in need thereof, and wherein upon oral administration, the prodrug or pharmaceutically acceptable salt minimizes, if not completely avoids, the gastrointestinal side effects usually seen after oral administration of the unbound mexiletine. The amount of the mexiletine is preferably a therapeutically effective amount. The disorder may be one treatable with mexiletine. For example, the disorder may be neuropathic myotonic or dystonic conditions. In a further embodiment, the GI side effect associated with administration of mexiletine is selected from, but is not limited to, emesis, nausea and abdominal discomfort.

The mexiletine prodrugs described herein may induce statistically significant lower average (e.g., mean) adverse effects on gut motility in the gastrointestinal environment as compared to a non-prodrug mexiletine salt form such as mexiletine HCl.

In an alternative aspect of the invention, a method for improving the pharmacokinetics and extending the duration of action of mexiletine in a subject in need thereof is provided. The method comprises administering to a subject in need thereof an effective amount of a prodrug of the present invention, or a composition thereof, wherein the plasma concentration time profile is modulated to minimize an initial upsurge in concentration of mexiletine, minimizing any consequential unwanted effects such as dizziness, while significantly extending the time for which the drug persists in plasma (resulting from continuing generation from the prodrug) and hence duration of action.

In a further aspect, a method for reducing inter- or intra-subject variability of mexiletine plasma levels is provided. The method comprises administering to a subject, or group of subjects in need thereof, an effective amount of a prodrug of the present invention, or a composition thereof.

In a further embodiment, a prodrug of p-OH or m-OH mexiletine is used in the method.

In another embodiment, a method is provided for eliminating, reducing or treating myotonic or dystonic conditions. The method comprises orally administering a mexiletine prodrug of the present invention, pharmaceutically acceptable salt thereof, or composition thereof, to a subject in need thereof. The amount of the mexiletine is preferably a therapeutically effective amount.

In a further embodiment, a prodrug of p-OH or m-OH mexiletine is used in the method.

Another embodiment of the invention is directed to reducing the inter- and intra-subject variability of mexiletine serum levels. The method comprises orally administering a mexiletine prodrug of the present invention, pharmaceutically acceptable salt thereof, or composition thereof, to a subject in need thereof. The amount of the mexiletine is preferably a therapeutically effective amount

Yet another embodiment of the invention related to increasing the reproducibility of the bioavailability of mexiletine, in a subject in need thereof. The method comprises orally administering a mexiletine prodrug of the present invention, pharmaceutically acceptable salt thereof, or composition thereof, to a subject in need thereof. The amount of the mexiletine is preferably a therapeutically effective amount.

In a further embodiment, a prodrug of p-OH mexiletine is used in the method.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### EXAMPLES

The present invention is further illustrated by reference to the following Examples. However, it should be noted that these Examples, like the embodiments described above, are illustrative and are not to be construed as restricting the enabled scope of the invention in any way.

### General Synthesis Procedures

The synthesis of a mexiletine prodrug of the present invention can be achieved in two distinct steps. An activated ester of an amino acid or peptide, for example, the activated ester of (S)-lysine, N,N′-di-t-butyloxycarbonyl-(S)-lysine succinimide, can be coupled to (rac)-mexiletine hydrochloride to yield the N-protected prodrug, (rac)-mexiletine-N,N′-di-t-butyloxycarbonyl-(S)-lysine. The compound can then be deprotected with trifluoroacetic acid to yield the prodrug.

As stated above, the activated lysine can be readily substituted for another activated amino acid or peptide

### EXAMPLES

### General Procedure for Preparation of Amino Acid Mexiletine Prodrugs

To mexiletine hydrochloride (1 mole equiv.) and 4-methylmorpholine (2.1 mole equiv.) in dry DMF (5 mL) was added N-Boc-(S)-amino acid N-hydroxysuccinimide ester (1.1 mole equiv.) and the resulting solution stirred at room temperature overnight. Ethyl acetate (50 mL) was then added and the solution quenched [NaCl: AcOH: H₂O; 0.45 g: 0.05 g: 180 mL] (50 mL) with stirring for 30 minutes. The organics were collected and quenched (50 mL) again for a further 30 minutes. After this time, the organics were collected and washed with 8% aqueous sodium bicarbonate (50 mL), 7% brine (50 mL), dried (MgSO₄) and concentrated. The resulting solid was either used without any further purification

To the N-Boc-(S)-amino acid-mexiletine (1 mole equiv.) was added 4M hydrogen chloride in dioxane (10 mole equiv.) and the resulting solution stirred at room temperature for 1 hour, then evaporated to dryness to afford the corresponding mexiletine-(S)-amino acid hydrochloride.

### Example 1

### Synthesis of (Rac)-Mexiletine-(S)-Lysine Ditrifluoroacetate

The synthesis of mexiletine-(S)-lysine-ditrifluoroacetate was achieved in two distinct steps as shown in the scheme below. Initially, the activated ester of (S)-lysine, N,N′-di-t-butyloxycarbonyl-(S)-lysine succinimide, was coupled to (rac)-mexiletine hydrochloride in the presence of N-methylmorpholine (NMM) to yield the N-protected prodrug, (rac)-mexiletine-N,N′-di-t-butyloxycarbonyl-(S)-lysine, after purification by chromatography (Scheme 1).

Subsequent deprotection of the BOC groups was then achieved using trifluoroacetic acid to give the desired (rac)-mexiletine-(S)-lysine-ditrifluoroacetate as a viscous glassy oil. The oil was found to foam on drying under high vacuum, but collapsed on standing in air. For the purposes of clarity, only one enantiomer of mexiletine is shown.

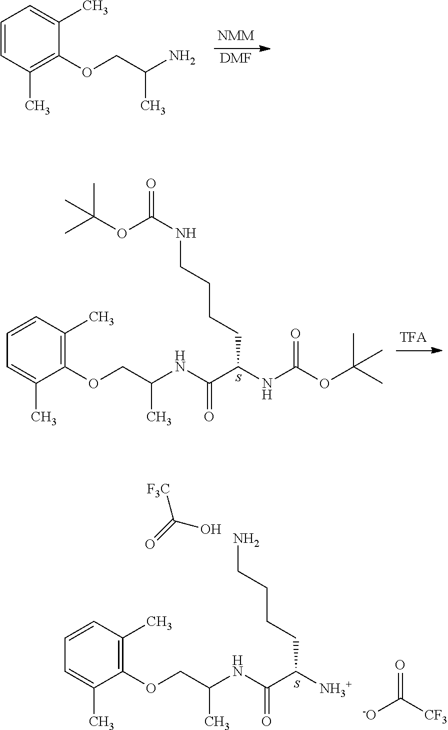

### Synthetic Route for (Rac)-Mexiletine(S)-Lysine Ditrifluoroacetate

### Detail

To (rac)-mexiletine HCl (200 mg, 0.93 mmol) and 4-methylmorpholine (214 μL, 197 mg, 1.95 mmol) in dry DMF (5 mL) was added N,N′-di-t-butyloxycarbonyl-(S)-lysine succinimide (452 mg, 1.02 mmol) and the solution stirred at room temperature overnight. Ethyl acetate (50 mL) was added and the solution quenched [NaCl: AcOH: H₂O; 0.45 g: 0.05 g: 180 mL] (50 mL) with stirring for 30 minutes. The organics were collected and quenched (50 mL) again for a further 30 minutes. After this time, the organics were collected and washed with 8% aqueous sodium bicarbonate (50 mL), 7% brine (50 mL), dried (MgSO₄) and concentrated. The resulting solid was chromatographed on silica gel eluting with ethyl acetate:petrol (4:6) to give N,N′-di-t-butyloxycarbonyl-(S)-lysine-(rac)-mexiletine (411 mg, 87%), as a white solid.

To the N,N′-di-t-butyloxycarbonyl-(S)-lysine-(rac)-mexiletine (411 mg, 0.81 mmol) was added trifluoroacetic acid (8 mL) and the resulting solution stirred at room temperature for 30 mins, then evaporated to dryness and stripped with chloroform (5×30 mL) to afford the (S)-lysine-(rac)-mexiletine di-trifluoroacetate (328 mg, 76%), as a viscous brown oil that foamed on drying in vacuo and then collapsed on exposure to air.

¹H NMR (DMSO-d₆) spectrum

8.60 (m, 1H, NH), 8.16 (br, 3H, NH₃⁺), 7.76 (br, 3H, NH₃⁺), 7.02 (m, 2H, ArH), 6.92 (m, 1H, ArH), 4.22 (m, 1H, α-CH), 3.67 (d, J=4.5 Hz, CH₂), 2.73 (m, 2H, NCH₂), 2.21 (s, 6H, 2×CH₃), 1.73 (m, 2H, CH₂), 1.52 (m, 2H, CH₂), 1.30 (m, 5H, CH₃+CH₂).

### Example 2

### Synthesis of (Rac)-Mexiletine-Glycine Trifluoroacetate

N-t-butyloxycarbonyl-glycine succinimide was coupled to (rac)-mexiletine hydrochloride in the presence of NMM, to yield the N-protected prodrug, (rac)-mexiletine-N-t-butyloxycarbonyl-glycine in good yield after purification by chromatograph (see scheme below)

Subsequent deprotection of the BOC groups was then achieved using trifluoroacetic acid. Trituration with diethyl ether and filtration gave the required (rac)-mexiletine glycine trifluoroacetate as a white solid in excellent yield (see scheme below). Note, for the purposes of clarity, only one enantiomer of mexiletine is shown in the scheme below.

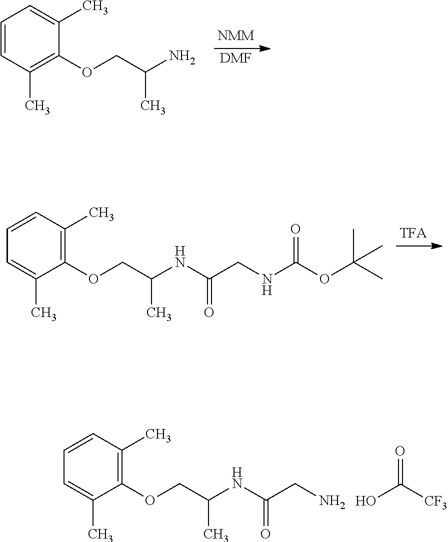

### Synthetic route for glycine-(rac)-mexiletine trifluoroacetate

Subsequent deprotection of the BOC groups was achieved using trifluoroacetic acid and filtration from diethyl ether give glycine-(rac)-mexiletine trifluoroacetate as a white solid in excellent yield.

### Detail

To (rac)-mexiletine hydrochloride (3.08 g, 14.24 mmol) and 4-methylmorpholine (3.3 L, 3.03 g, 29.9 mmol) in dry DMF (60 mL) was added N-t-butyloxycarbonyl-glycine succinimide (4.27 g, 15.67 mmol) and the solution stirred at room temperature overnight. Ethyl acetate (100 mL) was added and the solution quenched [NaCl: AcOH: H₂O; 0.45 g: 0.05 g: 180 mL] (100 mL) with stirring for 30 minutes. The organics were collected and quenched (100 mL) again for a further period of 30 minutes. After this time, the organics were collected and washed with 8% aqueous sodium bicarbonate (100 mL), 7% brine (100 mL), dried (MgSO₄) and concentrated to give the desired N-t-butyloxycarbonyl-glycine-(rac)-mexiletine (4.92 g, 93%), as a white solid.

To N-t-butyloxycarbonyl-glycine-(rac)-mexiletine (4.90 g, 14.58 mmol) was added trifluoroacetic acid (30 mL) and the resulting solution stirred at room temperature for 30 minutes, then evaporated to dryness and stripped with chloroform (5×30 mL). Diethyl ether was added and the resulting solid collected by filtration to afford the glycine-(rac)-mexiletine trifluoroacetate (4.98 g, 97%), as a white solid.

¹H NMR (DMSO-d₆) spectrum

8.52 (d, J=7.8 Hz, 1H, NH), 8.03 (br, 3H, NH₃⁺), 7.01 (m, 2H, ArH), 6.93 (m, 1H, ArH), 3.64 (m, 4H, 2×CH₂), 2.22 (s, 6H, 2×CH₃), 1.28 (d, J=6.6 Hz, 3H, CH₃).

### Example 3

### Synthesis of Mexiletine-(S)-Homoarginine Amide Dihydrochloride

The synthesis of mexiletine-(S)-homoarginine amide dihydrochloride was accomplished in five distinct steps as shown in the scheme below. The ‘activated ester’ N-Boc-(S)-homoarginine-(NO₂) N-hydroxysuccinimide ester was made via a DCC coupling between N-hydroxysuccinimide and N-Boc-(S)-homoarginine-(NO₂). Subsequent reaction with mexiletine hydrochloride yielded the N-protected prodrug, N-Boc-(S)-homoarginine-(NO₂)-mexiletine in good yield after purification using a Biotage Isolera automated chromatography system under reversed-phase conditions.

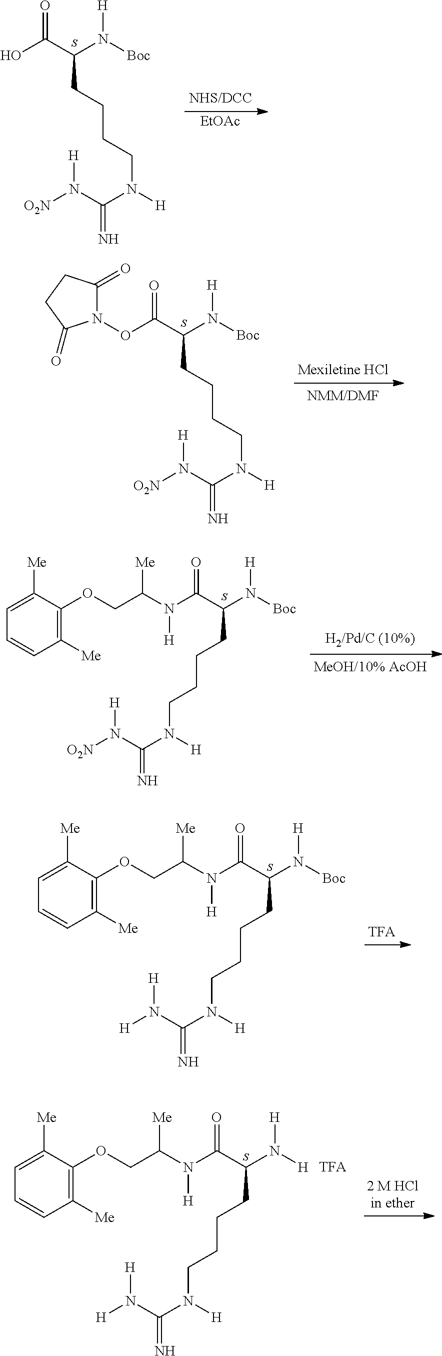

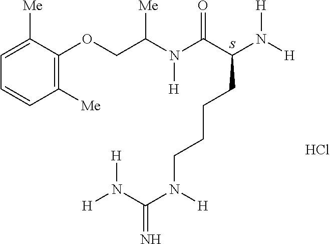

### Synthetic route for mexiletine-(S)-homoarginine amide dihydrochloride

The nitro-group was reduced via catalytic hydrogenation using palladium on carbon to give N-Boc-(S)-homoarginine-mexiletine. Removal of the Boc group was accomplished with trifluoroacetic acid. The crude product was subjected to salt exchange with 2M hydrogen chloride in diethyl ether and purified using a Biotage Isolera chromatography system under reversed-phase conditions to afford mexiletine-(S)-homoarginine amide dihydrochloride, as a white glassy solid

### Detail

To N-Boc-(S)-homoarginine-(NO₂)—OH (500 mg, 1.5 mmol) and N-hydroxysuccinimide (190 mg, 1.65 mmol) in ethyl acetate (50 mL) at 0° C. was added N,N′-dicyclohexylcarbodiimide (340 mg, 1.65 mmol) and the mixture was stirred at this temperature for 2 hours followed by room temperature overnight. The resulting suspension was filtered through Celite and the filtrate concentrated to give N-Boc-(S)-homoarginine-(NO₂) N-hydroxysuccinimide ester (621 mg, 100%), as a white solid which was used in the next reaction step without further purification

To mexiletine hydrochloride (302 mg, 1.4 mmol) in anhydrous DMF (20 mL) was added N-methylmorpholine (0.33 mL, 3.0 mmol) followed by N-Boc-(S)-homoarginine-(NO₂) N-hydroxysuccinimide ester (621 mg, 1.5 mmol) and the resulting mixture was stirred at room temperature overnight. The solution was diluted with ethyl acetate (100 mL) and quenched with [NaCl: AcOH: H₂O; 0.45 g: 0.05 g: 180 mL] (100 mL) with stirring for 30 minutes. The organics were washed with 8% aqueous sodium bicarbonate carbonate (100 mL), 7% brine (100 mL), dried (MgSO₄) and then concentrated to give a green oil which was purified using Biotage Isolera automated chromatography system under reversed-phase conditions; acetonitrile: H₂O (0.02% HCl), to give N-Boc-(S)-homoarginine-(NO₂)-mexiletine (462 mg, 54%), as a white solid.

N-Boc-(S)-homoarginine-(NO₂)-mexiletine (462 mg, 0.81 mmol) was added to 10% palladium on carbon (230 mg, 50% w/w) in methanol (12 mL) containing 10% glacial acetic acid (1.2 ml). The mixture was stirred under a hydrogen atmosphere for 24 hours. After this time, the reaction mixture was filtered through Celite and concentrated to yield N-Boc-(S)-homoarginine-mexiletine (542 mg, quantitative), as a white solid.

N-Boc-(S)-homoarginine-mexiletine (542 mg, 1.21 mmol) was dissolved in trifluoroacetic acid (10 mL) and stirred at room temperature for 45 minutes. After this time, the solution was concentrated and the remaining trifluoroacetic acid was removed azeotropically with chloroform (5×30 mL). The residue was stirred in 2 M hydrogen chloride in diethyl ether (5 mL) for 10 minutes, concentrated, and purified using a Biotage Isolera automated chromatography system under reversed-phase conditions; acetonitrile: H₂O (0.02% HCl) to facilitate salt exchange. Mexiletine-(S)-homoarginine amide dihydrochloride (300 mg, 59%) was isolated as a glassy white solid.

¹H NMR (DMSO-d₆) spectrum 8.79 (d, J=8.1 Hz, 1H, NH), 8.35 (br, 3H, NH₃⁺), 7.92 (m, 1H, NH), 7.02 (d, J=7.5 Hz, 2H, 2×ArH), 6.91 (m, 1H, ArH), 4.20 (m, 1H, α-CH), 3.82-3.65 (m, 3H, CH+OCH₂), 3.09 (m, 2H, E-CH₂), 2.22 (s, 6H, 2×CH₃), 1.75 (m, 2H, CH₂), 1.49-1.37 (m, 4H, 2×CH₂), 1.28 (m, 3H, CH₃).

### Example 4

### Synthesis of Mexiletine-(S)-Glutamic Acid Amide Hydrochloride

The synthesis of mexiletine-(S)-glutamic acid amide hydrochloride was achieved in two distinct steps as shown below. Initially, the ‘activated ester’ of (S)-glutamic acid, N-Boc-(S)-glutamic acid (tert-butyl ester) N-hydroxysuccinimide ester, was coupled to mexiletine hydrochloride. This gave the protected prodrug, N-Boc-(S)-glutamic acid (tert-butyl ester)-mexiletine in good yield after purification by chromatography.

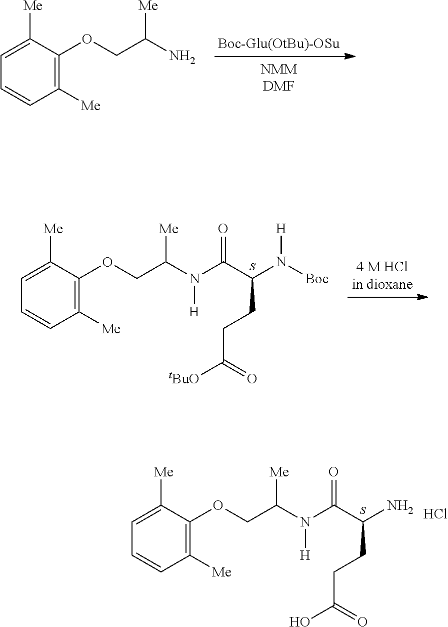

### Synthetic route for mexiletine-(S)-glutamic acid amide hydrochloride

Subsequent deprotection of the Boc and tert-butyl groups was achieved using a solution of 4M hydrogen chloride in dioxane. The crude product was purified using a Biotage Isolera automated chromatography system under reversed-phase conditions to afford the desired mexiletine-(S)-glutamic acid amide hydrochloride as a glassy white solid.

### Added Detail

Protected material: purified by medium pressure chromatography on silica eluting with ethyl acetate:petrol (30:70 v/v).

Final product: Biotage Isolera automated chromatography system under reversed-phase conditions: gradient of acetonitrile: H₂O (0.02% HCl).

Overall yield 190 mg, 34%

¹H NMR (DMSO-d₆) spectrum

8.76 (m, 1H, NH), 8.37 (s, 3H, NH₃⁺), 7.00 (d, J=7.5 Hz, 2H, 2×ArH), 6.90 (m, 1H, ArH), 4.20 (m, 1H, glutamic acid α-CH), 3.67 (m, 3H, obscured, mexiletine CH+OCH₂), 2.37 (m, 2 H, γ-CH₂), 2.21 (s, 3H, CH₃), 2.20 (s, 3H, CH₃), 1.99 (m, 2H, (3-CH₂), 1.26 (d, J=6.6 Hz, 3H, CH₃).

### Example 5

### Synthesis of Mexiletine-[(S)—S-Methyl-Methionine Chloride] Amide Hydrochloride

The synthesis of mexiletine-[(S)—S-methyl-methionine chloride] amide hydrochloride was achieved in three distinct steps as shown below. The ‘activated ester’ of (S)-methionine, N-Boc-(S)-methionine N-hydroxysuccinimide ester, was first coupled to mexiletine hydrochloride to yield the protected prodrug, N-Boc-(S)-methionine-mexiletine in good yield. Subsequent S-methylation was achieved using methyl iodide in methanol and the compound was purified by reversed-phase chromatography to give [N-Boc-(S)—S-methyl-methionine iodide]-mexiletine.

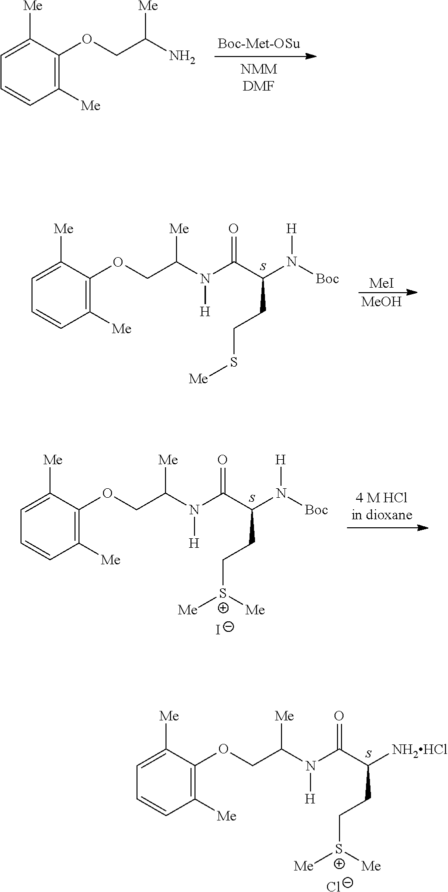

### Synthetic route for mexiletine-[(S)—S-methyl-methionine chloride] amide hydrochloride

Deprotection of the Boc group was carried out using 4 M hydrogen chloride in dioxane, followed by purification by reversed-phase chromatography, to afford the desired mexiletine-[(S)—S-methyl-methionine] amide hydrochloride.

### Detail

To mexiletine hydrochloride (0.75 g, 3.48 mmol) and 4-methylmorpholine (0.76 mL, 0.70 g, 6.96 mmol) in dry DMF (15 mL) was added N-Boc-(S)-methionine N-hydroxysuccinimide ester (1.00 g, 2.89 mmol) and the solution was stirred at room temperature overnight. Ethyl acetate (75 mL) was added and the solution quenched [NaCl: AcOH: H₂O; 0.45 g: 0.05 g: 180 mL] (150 mL) with stirring for 30 minutes. The organic layer was separated and washed with 8% aqueous sodium bicarbonate (150 mL), saturated brine (150 mL), dried (Na₂SO₄) and concentrated. The resulting white solid of N-Boc-(S)-methionine-mexiletine (1.10 g, 92%) was used in the next step without further purification.

The N-Boc-(S)-methionine-mexiletine (0.5 g, 1.22 mmol) was dissolved in methanol (10 mL) and methyl iodide (0.18 mL, 0.42 g, 2.93 mmol) was added dropwise. The resulting mixture was stirred at room temperature, with the progress of the reaction being followed by TLC (ethyl acetate: petrol; 6:4/Rf of s.m. 0.90 and product 0.0). After 5 days, the reaction was found to be complete and so the solvent was removed under vacuum to yield a crude yellow solid which was purified using a Biotage Isolera automated chromatography system under reversed-phase conditions (C₁₈ column, gradient of 0→100% MeCN in 0.02% hydrochloric acid) to give [N-Boc-(S)—S-methyl-methionine]-mexiletine (0.32 g, 62%), as a yellow solid.

To [N-Boc-(S)—S-methyl-methionine]mexiletine (0.32 g, 0.75 mmol) was added 4 M hydrogen chloride in dioxane (0.26 mL, 7.6 mmol) and the resulting solution was stirred at room temperature for 3 h. The mixture was evaporated to dryness and azeotropically co-evaporated with chloroform (5×10 mL) to afford a yellow solid. This solid was purified using a Biotage Isolera automated chromatography system under reversed-phase conditions (C₁₈ column, gradient of 0→100% MeCN in 0.02% hydrochloric acid) to give mexiletine-[(S)—S-methyl-methionine chloride] amide hydrochloride (50 mg, 18%) as a yellow solid.

¹H NMR (DMSO-d₆) spectrum

9.14 (m, 1H, NH), 8.63 (br, 3H, NH₃⁺), 7.02 (d, J=7.5 Hz, 2H, 2×ArH), 6.95 (m, 1H, ArH), 4.22 (m, 1H, α-CH), 4.03 (m, 1H, CH), 3.51-3.75 (m, 4H, CH₂+OCH₂), 2.96 (m, 6H, 2×S—CH₃), 2.31 (obscured, 2H, CH₂), 2.23 (s, 6H, 2×CH₃), 1.30 (m, 3H, CH₃).

### Example 6

### Synthesis of Mexiletine (Carboxymethyl-Glycine) Amide Hydrochloride

The synthesis of mexiletine (carboxymethyl-glycine) amide hydrochloride was achieved in three distinct steps. N-Boc-iminodiacetic acid was cyclised by treatment with N,N′-dicyclohexylcarbodi-imide and the resulting anhydride was subsequently ring-opened with mexiletine hydrochloride to yield the protected prodrug, mexiletine (N-Boc-carboxymethyl-glycine) amide after purification (see Scheme below).

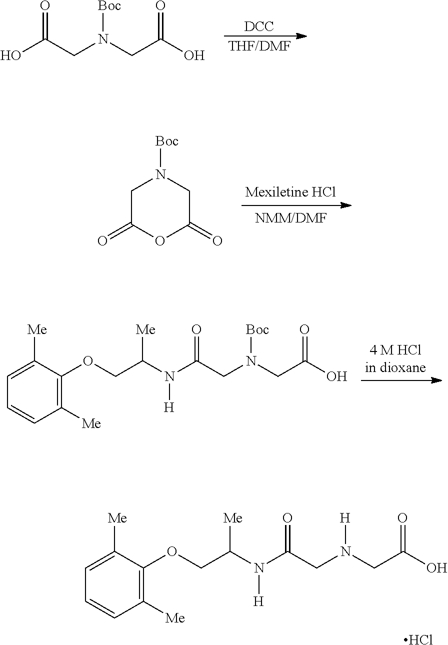

### Synthetic Route for Mexiletine (Carboxymethyl-Glycine) Amide Hydrochloride

Subsequent deprotection of the Boc group was achieved using 4 M hydrogen chloride in dioxane to give mexiletine (carboxymethyl-glycine) amide hydrochloride after purification by reversed-phase chromatography.

### Detail

To a stirred solution of N-Boc-iminodiacetic acid (2.0 g, 8.62 mmol) in dry THF (50 mL) and dry DMF (8 mL) was added N,N′-dicyclohexylcarbodi-imide (1.77 g, 8.62 mmol) and the mixture was stirred at room temperature for 5 h. Mexiletine hydrochloride (1.85 g, 8.62 mmol) and 4-methylmorpholine (0.94 mL, 868 mg, 8.62 mmol) in dry DMF (15 mL) were added to the mixture and stirring was continued at room temperature overnight. The resulting suspension was filtered through Celite and the THF was removed by evaporation. Water (100 mL) was added and the solution was extracted with ethyl acetate (2×20 mL). The combined organic layers were washed with water (5×100 ml), saturated brine (100 mL), dried (Na₂SO₄) and concentrated. The resulting solid was purified using a Biotage Isolera automated chromatography system under normal-phase conditions [silica column, gradient of 0→10% (methanol containing 0.1% Et₃N) in dichloromethane] to give N-Boc-mexiletine (carboxymethyl-glycine) amide (1.61 g, 48%), as a white solid.

To N-Boc-Mexiletine (carboxymethyl-glycine) amide (750 mg, 1.90 mmol) was added 4 M hydrogen chloride in dioxane (5 mL) and the resulting solution was stirred at room temperature for 2 h. The solution was evaporated to dryness and triturated with diethyl ether (5×10 mL) to afford a white solid. This solid was purified using a Biotage Isolera automated chromatography system under reversed-phase conditions (C₁₈ column, gradient of 0→100% MeCN in 0.02% hydrochloric acid) to give the required mexiletine (carboxymethyl-glycine) amide hydrochloride (326 mg, 53%).

¹H NMR (DMSO-d₆) spectrum

8.62 (d, J=8.1 Hz, 1H, NH), 7.02-7.00 (d, J=7.5 Hz, 2H, 2×ArH), 6.93-6.88 (m, 1H, 1×ArH), 4.21-4.17 (m, 1H, CH), 3.72-3.59 (m, 6H, 3×CH₂) 2.20 (s, 6H, 2×CH₃), 1.27-1.25 (d, J=6.0 Hz, 3H, CH₃)

### Example 7

### Synthesis of Mexiletine [(S)—N^{α}-Acetyl-Lysine] Amide Hydrochloride

The synthesis of mexiletine [(5)-1V′-acetyl-lysine] amide hydrochloride was achieved in three distinct steps (see Scheme below). Initially, N^{α}-acetyl-N^{ε}-Boc-(S)-lysine was coupled with N-hydroxysuccinimide in the presence of DCC to give the ‘activated ester’, N^{α}-acetyl-N^{ε}-Boc-(S)-lysine N-hydroxysuccinimide ester. This was coupled to mexiletine hydrochloride to yield the protected prodrug, N^{α}-acetyl-N^{ε}-Boc-(S)-lysine-mexiletine after purification by normal phase chromatography.

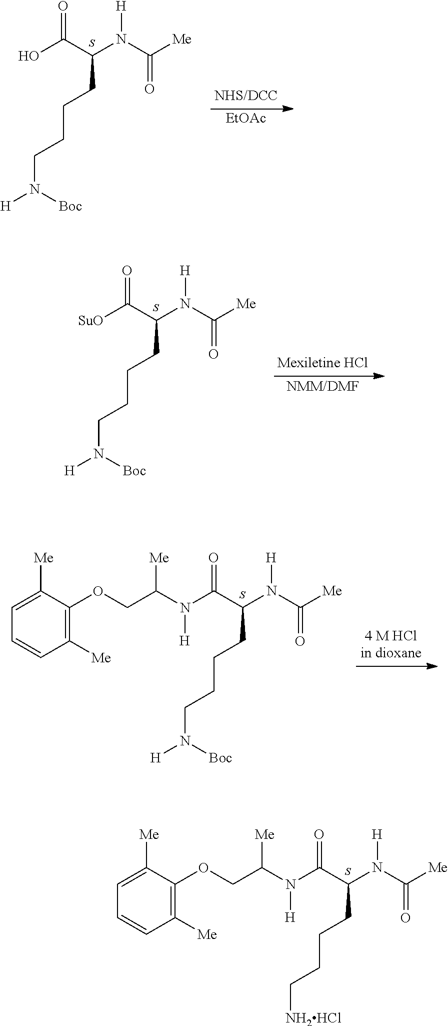

### Synthetic Route for Mexiletine [(S)—N^{α}-Acetyl-Lysine] Amide Hydrochloride

Subsequent deprotection of the Boc group was achieved using 4 M hydrogen chloride in dioxane, followed by purification using reversed-phase chromatography to afford mexiletine [(S)—N^{ε}-acetyl-lysine] amide hydrochloride.

### Detail

To a stirred solution of N^{α}-acetyl-N^{ε}-Boc-(S)-lysine (1.00 g, 3.47 mmol) and N-hydroxysuccinimide (0.44 g, 3.81 mmol) in ethyl acetate (25 mL) at 0° C. was added N,N′-dicyclohexylcarbodi-imide (0.79 g, 3.81 mmol) and the mixture was stirred at this temperature for 2 h and then at room temperature overnight. The resulting suspension was filtered through Celite and the filtrate was concentrated to give N^{α}-acetyl-N^{ε}-Boc-(S)-lysine N-hydroxysuccinimide ester (1.44 g, 100%), as a white solid which was used in the next step without further purification.

To mexiletine hydrochloride (0.82 g, 3.81 mmol) and 4-methylmorpholine (0.42 mL, 0.38 g, 3.81 mmol) in dry DMF (20 mL) was added N^{α}-acetyl-N^{ε}-Boc-(S)-lysine N-hydroxysuccinimide ester (1.44 g, 3.47 mmol) and the solution was stirred at room temperature overnight. Ethyl acetate (80 mL) was added and the solution quenched [NaCl: AcOH: H₂O; 0.45 g: 0.05 g: 180 mL] (160 mL) with stirring for 30 minutes. The organic layer was separated and washed with 8% aqueous sodium bicarbonate (160 mL), saturated brine (160 mL), dried (Na₂SO₄) and concentrated. The resulting crude white solid was purified by medium-pressure chromatography on silica eluting with dichloromethane—methanol (95:5) to yield N^{α}-acetyl-N^{ε}-Boc-(S)-lysine-mexiletine (1.50 g, 96%), as a white solid.

To N^{α}-acetyl-N^{ε}-Boc-(S)-lysine-mexiletine (1.50 g, 96%) was added 4 M hydrogen chloride in dioxane (1.21 mL, 33.3 mmol) and the resulting solution was stirred at room temperature for 3 h. The resulting mixture was evaporated to dryness and azeotropically co-evaporated with chloroform (5×20 mL) to give a white solid. This solid was purified using a Biotage Isolera automated chromatography system under reversed-phase conditions (C₁₈ column, gradient of 0→100% MeCN in 0.02% hydrochloric acid) to give mexiletine [(S)—N^{α}-acetyl-lysine amide] hydrochloride (0.94 g, 74%), as a white solid.

¹H NMR (DMSO-d₆) spectrum 8.09 (m, 5H, 2×NH+NH₃⁺), 6.99 (d, J=7.5 Hz, 2H, 2×ArH), 6.90 (m, 1H, ArH), 4.25 (m, 1H, CH), 4.10 (m, 1H, CH), 3.61 (m, 2H, CH₂), 2.70 (m, 2 H, CH₂), 2.19 (s, 6H, 2×Me), 1.85 (s, 1.5H, 0.5×CH₃), 1.84 (s, 1.5H, 0.5×CH₃), 1.54 (m, 4 H, 2×CH₂), 1.26 (m, 5H, CH₂+CH₃).

### Example 8

### Synthesis of Mexiletine-(S)-Aspartic Acid Amide Hydrochloride

The synthesis of mexiletine-(S)-aspartic acid amide hydrochloride was achieved in two reaction steps. N-Boc-(S)-aspartic acid(tert-butyl ester) N-hydroxysuccinimide ester was first coupled to mexiletine hydrochloride in the presence of 4-methylmorpholine (NMM) in DMF. This gave the protected prodrug, Boc-(S)-aspartic acid(tert-butyl ester)-mexiletine in good yield after purification by chromatography (see Scheme below).

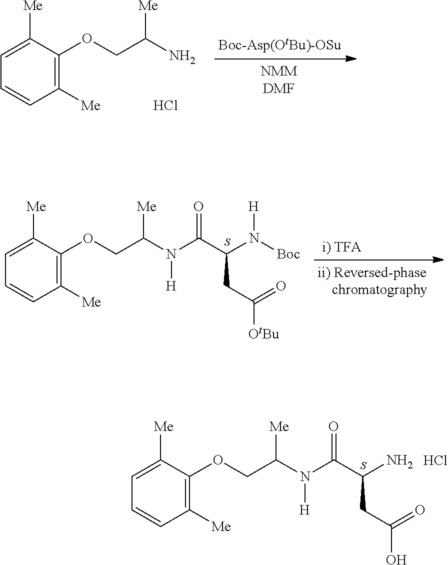

### Synthetic route for mexiletine-(S)-aspartic acid amide hydrochloride

Subsequent deprotection of the Boc and tert-butyl groups was achieved using trifluoroacetic acid. Reversed-phase chromatography (with dilute hydrochloric acid in the mobile phase) afforded the desired mexiletine-(S)-aspartic acid amide hydrochloride as a white glassy solid.

### Detail

To a stirred solution of mexiletine hydrochloride (0.50 g, 2.56 mmol) and 4-methylmorpholine (0.26 g, 0.36 mL, 2.56 mmol) in anhydrous DMF (15 mL) was added N-Boc-(S)-aspartic acid (tert-butyl ester) N-hydroxysuccinimide ester (0.99 g, 2.56 mmol) and stirring was continued at room temperature overnight. Ethyl acetate (100 mL) and water (100 mL) were added and the organic layer was separated, washed with water (4×100 mL), and brine (100 mL), dried (MgSO₄) and concentrated to afford a gummy semi-solid solid (1.32 g). The residue was purified using a Biotage Isolera automated chromatography system under normal phase conditions (silica column, gradient of 0→100% ethyl acetate in petrol) with detection at 254 nm to afford Boc-(S)-aspartic acid(tert-butyl ester)-mexiletine (1.03 g, 100%), as a colourless gummy semi-solid.

A solution of Boc-(S)-aspartic acid(tert-butyl ester)-mexiletine (1.03 g, 2.52 mmol) in trifluoroacetic acid (15 mL) was stirred at room temperature for 1 h. The mixture was evaporated to dryness and residual trifluoroacetic acid was removed azeotropically with chloroform (3×30 mL) to afford a white solid (612 mg). This solid residue was purified using a Biotage Isolera automated chromatography system under reversed-phase conditions (C₁₈ column, gradient of 0→100% MeCN in 0.02% hydrochloric acid) with detection at 263 nm to afford, after freeze-drying, mexiletine-(S)-aspartic acid amide hydrochloride (0.32 g, 33%), as a white glassy solid

¹H NMR (DMSO-d₆) spectrum

8.66 (dd, J=3.6, 11.7 Hz, 1H, NH), 7.02 (d, J=7.8 Hz, 2H, 2×ArH), 6.91 (m, 1H, ArH), 4.17 (m, 1H, α-CH), 4.03 (m, 1H, CH), 3.73-3.58 (m, 2H, CH₂), 2.87-2.81 (m, 2H, CH₂), 2.22 (s, 3H, CH₃), 2.21 (s, 3H, CH₃), 1.27 (m, 3H, CH₃)

### Example 9

### Synthesis of Mexiletine-Glycocyamine Amide Hydrochloride

The synthesis of mexiletine-glycocyamine amide hydrochloride was achieved in three steps (see Scheme below). Mexiletine glycine amide trifluoroacetate (see example 2) was reacted with 1,3-di-Boc-2-(trifluoromethylsulfonyl)guanidine to give the di-Boc-protected prodrug, mexiletine-(di-Boc-glycocyamine) amide, in good yield and purity after purification.

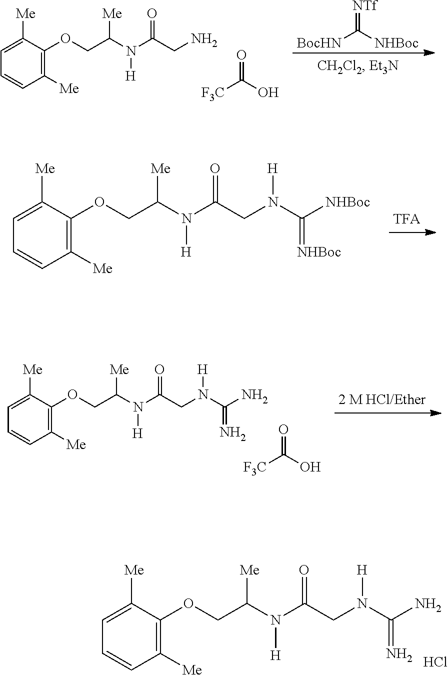

### Synthetic route for mexiletine-glycocyamine amide hydrochloride

Deprotection of the Boc groups was achieved using trifluoroacetic acid to yield the product as a trifluoroacetic acid salt which was subsequently converted to the hydrochloride salt, mexiletine-glycocyamine amide hydrochloride, as a white glassy solid after purification by reversed-phase chromatography.

### Detail

To a stirred suspension of mexiletine glycine amide trifluoroacetate (447 mg, 1.28 mmol) and 1,3-di-Boc-2-(trifluoromethylsulfonyl)guanidine (1.00 g, 2.56 mmol) in anhydrous dichloromethane (10 mL) was added triethylamine (259 mg, 0.36 mL, 2.56 mmol) and stirring was continued for 17 h. The reaction mixture was diluted with dichloromethane (50 mL) and quenched with saturated aqueous NaHCO₃ (50 mL) with stirring for 30 min. The organic layers separated, washed with water (2×50 mL), saturated brine (50 mL), dried (MgSO₄) and concentrated to give a white semi-solid. The residue was purified using a Biotage Isolera automated chromatography system under normal phase conditions (silica column, gradient of 0→40% EtOAc in petrol) with detection at 254 nm to afford mexiletine-(di-Boc-glycocyamine) amide (598 mg, 70%) as a colourless oil.

Mexiletine-(di-Boc-glycocyamine) amide (598 mg, 1.25 mmol) in trifluoroacetic acid (20 mL) was stirred at room temperature for 45 min. The mixture was evaporated to dryness and residual trifluoroacetic acid was removed azeotropically with chloroform (5×20 mL) to give a white solid which was suspended in 2 M HCl in diethyl ether with stirring for 20 min. The suspension was concentrated to dryness and the residue was purified using a Biotage Isolera automated chromatography system under reversed-phase conditions (C₁₈ column, gradient of 0→100% MeCN in 0.02% aqueous HCl) with detection at 263 nm to afford, after freeze-drying, mexiletine-glycocyamine amide dihydrochloride (234 mg, 60%) as a glassy white solid.

¹H NMR (DMSO-d₆) spectrum

8.33 (d, J=8.1 Hz, 1H, NH), 7.61 (t, J=5.7 Hz, 1H, NH), 7.31 (br, 3H, NH₃⁺), 7.01 (d, J=7.5 Hz, 2H, 2×ArH), 6.90 (m, 1H, ArH), 4.17 (m, 1H, □-CH), 3.85 (m, 2H, OCH₂), 3.64 (m, 2H, CH₂), 2.21 (s, 6H, 2×CH₃), 1.27 (d, J=6.9 Hz, 3H, CH₃).

### Example 10

### Synthesis of Mexiletine-(S)—N-Methylarginine Amide Dihydrochloride

The synthesis of mexiletine-(S)—N-methylarginine amide dihydrochloride was achieved in seven distinct steps. Initially, N-Boc-(S)-ornithine(Cbz)-OH was converted to the ‘activated ester’ through reaction with N-hydroxysuccinimide. The ‘activated ester’ N-Boc-(S)-ornithine(Cbz) N-hydroxysuccinimide ester was coupled to mexiletine hydrochloride to yield, mexiletine [N-Boc-(S)-ornithine(Cbz)] amide after purification by chromatography. The Cbz group was removed via catalytic hydrogenolysis using palladium on carbon to give mexiletine [N-Boc-(S)-ornithine] amide (Scheme below).

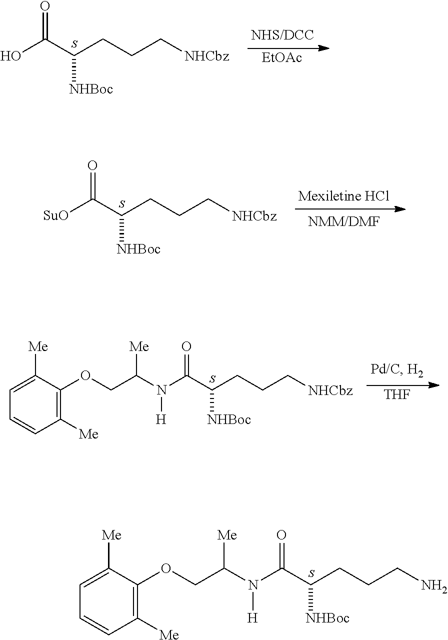

### Synthesis of Mexiletine [N-Boc-(S)-Ornithine] Amide

Mexiletine [N-Boc-(S)-ornithine] amide was then reacted with di(imidazole-1-yl)methanimine to give an ‘activated guanidine’. The synthesis of di(imidazole-1-yl)methanimine was achieved through reaction of imidazole with cyanogen bromide (BrCN) in one step (Scheme below).

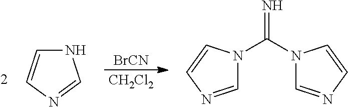

### Synthesis of Di(Imidazole-1-yl)Methanimine

Replacement of the imidazole moiety with methylamine was accomplished via the use of a microwave and irradiating the reaction mixture for 30 minutes at 120° C. with one equivalent of trifluoroacetic acid to afford mexiletine [N-Boc-N—(S)-methylarginine] amide (Scheme below).

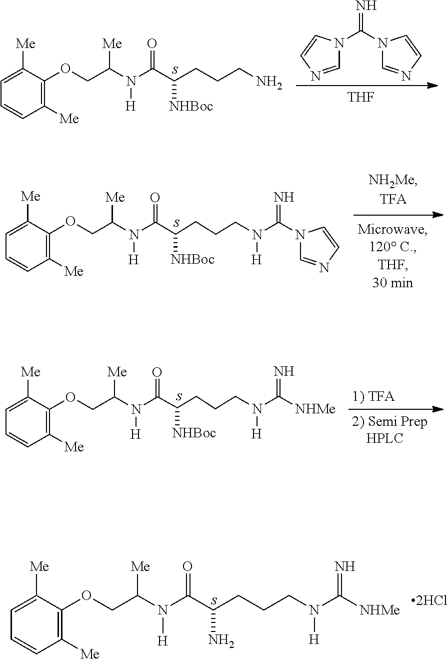

### Synthesis of Mexiletine-(S)—N-Methylarginine Amide Dihydrochloride

Subsequent deprotection of the Boc group was achieved using trifluoroacetic acid. The residue was purified using semi-preparative HPLC with HCl in the eluent to afford mexiletine-(S)—N-methylarginine amide dihydrochloride as a white glassy solid.

### Detail

To a stirred solution of N-Boc-(S)-ornithine(Cbz)-OH (10.0 g, 27.0 mmol) and N-hydroxysuccinimide (3.45 g, 30.0 mmol) in ethyl acetate (100 mL) at 0° C. was added N,N′-dicyclohexylcarbodiimide (5.90 g, 28.0 mmol) in one portion, and stirring was continued overnight during which the mixture was allowed to warm to room temperature. The resulting suspension was filtered through Celite and the filtrate was concentrated to afford N-Boc-(S)-ornithine(Cbz) N-hydroxysuccinimide ester as a colourless gummy semi-solid (9.74 g, 78%) which was used without further purification.

To a stirred solution of mexiletine hydrochloride (2.11 g, 9.80 mmol) and 4-methylmorpholine (1.09 g, 1.19 mL, 11.0 mmol) in anhydrous DMF (50 mL) was added N-Boc-(S)-ornithine(Cbz) N-hydroxysuccinimide ester (5.00 g, 11.0 mmol) and stirring was continued at room temperature overnight. Ethyl acetate (100 mL) and water (100 mL) were added and the organic layer was separated, washed with water (5×100 mL), saturated brine (100 mL), dried (MgSO₄) and concentrated to afford mexiletine [N-Boc-(S)-ornithine(Cbz)] amide (2.10 g, 41%) as a white solid which was used without further purification.

10% Palladium on carbon (1.05 g) was cautiously wetted with anhydrous THF (40 mL) under nitrogen. A solution of mexiletine [N-Boc-(S)-ornithine(Cbz)] amide (2.10 g, 4.00 mmol) in anhydrous THF (20 mL) was added, and the flask was evacuated. An atmosphere of hydrogen was introduced via a balloon, and the mixture was stirred for 2 h at room temperature. The catalyst was removed by filtration of the suspension through a thin layer of Celite, and the filtrate was concentrated to afford mexiletine [N-Boc-(S)-ornithine] amide (1.28 g, 44%) as a colourless gummy semi-solid which was used without further purification.

To a stirred solution of imidazole (6.80 g, 100 mmol) in anhydrous dichloromethane (500 mL) was added BrCN (3.70 g, 33 mmol) and stirring was continued at reflux for 30 min. The mixture was cooled to room temperature and concentrated to afford di(imidazole-1-yl)methanimine (4.05 g, 72%) as a pale yellow solid which was used without further purification.

¹H NMR (DMSO-d₆) spectrum

10.21 (br s, 1H, NH), 8.09 (s, 2H, 2×CH), 7.57 (s, 2H, 2×CH), 7.12 (s, 2H, 2×CH).

To a stirred solution of di(imidazole-1-yl)methanimine (0.50 g, 3.25 mmol) in anhydrous tetrahydrofuran (15 mL) was added mexiletine [N-Boc-(S)-ornithine] amide (1.28 g, 3.25 mmol) and stirring was continued at room temperature overnight. The mixture was evaporated to dryness. Dichloromethane (100 mL) and water (100 mL) were added and the organic layer was separated, washed with saturated aqueous ammonium chloride (5×100 mL), saturated brine (100 mL), dried (MgSO₄) and concentrated. The residue was triturated with diethyl ether, collected by suction filtration and dried in vacuo to afford mexiletine [N^{α}-Boc-N⁵-imidazole-1-yl-(S)-arginine] amide (0.63 g, 40%) as a white solid.

To a microwave vial containing mexiletine [N^{α}-Boc-N^{δ}-imidazole-1-yl-(S)-arginine]amide (0.63 g, 1.24 mmol) in anhydrous tetrahydrofuran (2 mL) was added trifluoroacetic acid (0.14 g, 92 μL, 1.24 mmol) and 2 M dimethylamine in tetrahydrofuran (10 mL, 20 mmol). The microwave vial was capped and irradiated for 30 minutes at 120° C. in a microwave. The vial was decapped and the reaction mixture evaporated to dryness. The residue was purified by medium-pressure chromatography on silica eluting with a gradient of 1→15% MeOH in dichloromethane to afford mexiletine [N-Boc-(S)—N-methylarginine] amide (0.10 g, 17%) as a clear gummy semi-solid. R_{f} 0.16 (10% MeOH—90% dichloromethane).

Mexiletine [N-Boc-(S)—N-methylarginine] amide (0.10 g, 0.22 mmol) in trifluoroacetic acid (2 mL) was stirred at room temperature for 20 min. The mixture was evaporated to dryness and residual trifluoroacetic acid was removed azeotropically with chloroform (2×20 mL) to afford a gummy semi-solid (122 mg). The impure material was dissolved in MeCN:H₂O (1:1) to give a concentration of 153 mg/mL. This solution was purified by semi-preparative HPLC injecting 100 μl portions and collecting the eluent containing the pure substance. The combined fractions were reduced in volume by removing the acetonitrile and most of the water and finally lyophilized to give mexiletine-(S)—N-methylarginine amide dihydrochloride (33 mg, 35%) as a white glassy solid.

¹H NMR (DMSO-d₆) spectrum

8.86 (d, J=7.8 Hz, 1H, NH), 8.35 (s, 3H, NH₃⁺), 7.82 (m, 1H, NH), 7.66 (m, 1H, NH), 7.47 (d, J=11.4 Hz, 2H, NH₂⁺), 7.02 (d, J=7.5 Hz, 2H, 2×ArH), 6.91 (m, 1H, ArH), 4.22 (m, 1 H, CH), 3.79 (m, 3H, CH₂+CH), 3.18 (m, 2H, CH₂), 2.74 (d, J=4.8 Hz, 1.5H, 0.5×CH₃), 2.69 (d, J=4.5 Hz, 1.5H, 0.5×CH₃), 2.23 (s, 3H, CH₃), 2.17 (s, 3H, CH₃), 1.77 (m, 2H, CH₂), 1.55 (m, 2H, CH₂), 1.29 (m, 3H, CH₃)

### Example 11

### Synthesis of Mexiletine-(S)—N,N-Dimethylarginine Amide Dihydrochloride

The synthesis of mexiletine-(S)—N,N-dimethylarginine amide dihydrochloride was achieved in an analogous manner to that of mexiletine-(S)—N-methylarginine amide dihydrochloride. The divergent point in the synthesis occurred when the imidazole moiety in mexiletine [N^{α}-Boc-N^{δ}-imidazole-1-yl-(S)-arginine] amide (see section 10) was replaced with dimethylamine instead of methylamine. This again was accomplished via the use of a microwave, irradiating the reaction mixture for 30 minutes at 120° C. with one equivalent of trifluoroacetic acid to afford mexiletine [N-Boc-(S)—N-dimethylarginine] amide (see scheme below).

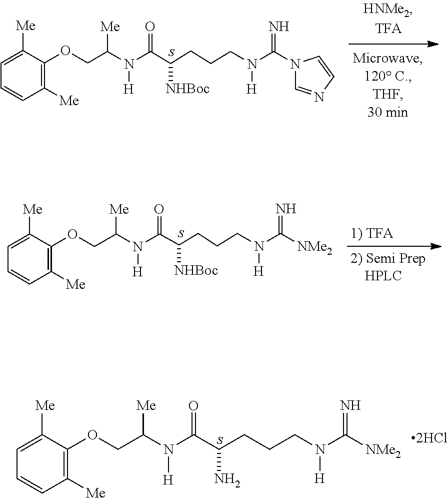

### Synthesis of Mexiletine-(S)—N,N-Dimethylarginine Amide Dihydrochloride

Subsequent deprotection of the Boc group was achieved using trifluoroacetic acid. The residue was purified using semi-preparative HPLC with HCl in the eluent to afford mexiletine-(S)—N,N-dimethylarginine amide dihydrochloride) as a white glassy solid.

### Detail

To a microwave vial containing mexiletine [N^{α}-Boc-N^{δ}-imidazole-1-yl-(S)-arginine]amide (0.60 g, 1.24 mmol) in anhydrous THF (2 mL) was added trifluoroacetic acid (0.14 g, 96 μL, 1.29 mmol) and 2 M dimethylamine in tetrahydrofuran (10 mL, 20 mmol). The vial was capped and irradiated for 30 minutes at 120° C. in a microwave. The vial was decapped and the reaction mixture evaporated to dryness. The residue was purified by medium-pressure chromatography on silica eluting with a gradient of 1→15% MeOH in dichloromethane to afford mexiletine [N-Boc-(S)N,N-dimethylarginine] amide (0.32 g, 55%) as a clear gummy semi-solid. R_{f} 0.18 [10% (MeOH—90% dichloromethane].

Mexiletine [N-Boc-(S)—N,N-dimethylarginine] amide (0.32 g, 0.69 mmol) in trifluoroacetic acid (4 mL) was stirred at room temperature for 45 min. The mixture was evaporated to dryness and residual trifluoroacetic acid was removed azeotropically with chloroform (5×10 mL) to afford a gummy semi-solid (315 mg). The impure material was dissolved in MeCN:H₂O (1:0.5) to give a concentration of 94.5 mg/mL. This solution was purified by semi-preparative HPLC injecting 100 μl portions and collecting the eluent containing the pure substance. The combined fractions were reduced in volume by removing the acetonitrile and most of the water and finally lyophilized to give mexiletine-(S)—N,N-dimethylarginine amide dihydrochloride (61 mg, 21%) as a white glassy solid.

¹H NMR (DMSO-d₆) spectrum

8.97 (d, J=8.4 Hz, 1H, NH), 8.40 (s, 3H, NH₃⁺), 7.72 (m, 1H, NH), 7.47 (d, J=11.7 Hz, 2H, NH₂⁺), 7.01 (d, J=7.5 Hz, 2H, 2×ArH), 6.91 (m, 1H, ArH), 4.21 (m, 1H, CH), 3.76 (m, 3H, CH₂+CH), 3.26 (m, 2H, CH₂), 2.74 (s, 3H, CH₃), 2.92 (s, 3H, CH₃), 2.23 (s, 3H, CH₃), 2.20 (s, 3H, CH₃), 1.77 (m, 2H, CH₂), 1.58 (m, 2H, CH₂), 1.27 (m, 3H, CH₃)

### Example 12

### Synthesis of Mexiletine [(R)—S-Methyl-Cysteine Sulfoxide] Amide Trifluoroacetate

The synthesis of mexiletine [(R)—S-methyl-cysteine sulfoxide] amide trifluoroacetate was achieved in two steps. N-Boc-mexiletine [(R)—S-methyl-cysteine] (see below) was oxidised with m-chloroperoxybenzoic acid to yield N-Boc-mexiletine [(R)—S-methyl-cysteine sulfoxide] amide after purification by normal phase chromatography (see Scheme below).

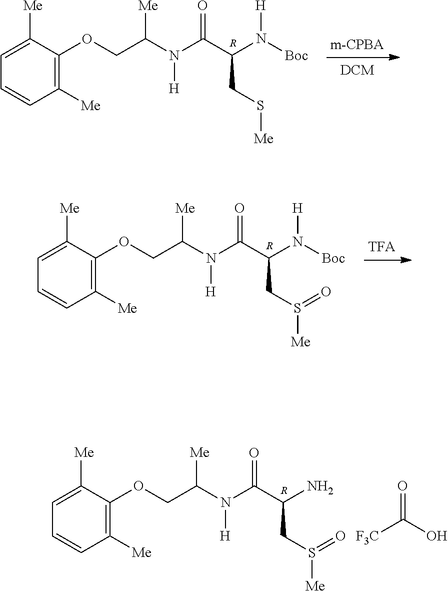

### Synthetic route for mexiletine [(R)—S-methyl-cysteine sulfoxide] amide trifluoroacetate

Subsequent deprotection of the Boc group was achieved using trifluoroacetic acid to afford mexiletine [(R)—S-methyl-cysteine sulfoxide] amide trifluoroacetate

### Detail

To a solution of mexiletine-[(R)—S-methyl-cysteine] amide (700 mg, 1.77 mmol) in dichloromethane (15 mL) was added m-chloroperoxybenzoic acid (319 mg, 1.85 mmol), and the resulting mixture was stirred for 5 h at room temperature. Saturated aqueous sodium bisulphate (10 mL) was added to the mixture, the layers were separated and the organic layer was washed with saturated aqueous saturated aqueous sodium bicarbonate (40 mL), brine (40 mL), dried (MgSO₄) and concentrated. The resulting crude solid was purified using a Biotage Isolera automated chromatography system under normal-phase conditions [silica column, gradient of 0→10% (methanol containing 0.1% Et₃N) in dichloromethane] to give N-Boc-mexiletine [(R)—S-methyl-cysteine sulfoxide] amide (504 mg, 69%).

To N-Boc-Mexiletine [(R)—S-methyl-cysteine sulfoxide] amide (200 mg, 0.48 mmol) was added trifluoroacetic acid (3 mL) and the resulting solution was stirred at room temperature for 20 min. The mixture was evaporated to dryness and residual trifluoroacetic acid was removed azeotropically with chloroform (5×20 mL). The residue was triturated with diethyl ether (3×20 mL) to afford mexiletine [(R)—S-methyl-cysteine sulfoxide] amide trifluoroacetate (187 mg, 91%), as a glassy white solid.

¹H NMR (DMSO-d₆) spectrum

8.79 (m, 1H, NH), 8.40 (br, 3H, NH₃⁺), 7.03-7.00 (d, J=9.0 Hz, 2H, 2×ArH), 6.91-6.89 (m, 1H, 1×ArH), 4.26-4.20 (m, 2H, α-CH+CH), 3.71-3.63 (m, 2H, OCH₂), 3.20-3.04 (m, 2H, SCH₂), 2.71 (m, 3H, SCH₃), 2.22 (m, 6H, 2×CH₃), 1.31-1.26 (m, 3H, CH₃).

### Example 13

### In Vitro Stability of Mexiletine Prodrugs Under Conditions Prevailing in the Gut

Inherent chemical and biological stability of the mexiletine prodrugs of the present invention, in the conditions prevailing in the GI tract, is an important requirement. If a prodrug is prematurely hydrolyzed, the active drug molecule would be released and could exert a local anaesthetic action within the stomach so giving rise to gastric stasis and emesis

### Methodology

To investigate if the prodrugs of the present invention are stable in conditions mimicking the gut, various mexiletine amino acid prodrugs were incubated at 37° C. in simulated gastric and simulated intestinal juice (USP defined composition) for 2 hours. The remaining concentrations of the prodrug were then assayed by HPLC.

### Results

As can be seen in Table 2, all of these prodrug conjugates tended to be very stable under the stimulated conditions existing in the GI tract. Thus, these prodrugs would not be expected to exert any local anaesthetic activity as the result of release of the active drug in the stomach. However any inherent local anesthetic activity of the prodrugs could potentially have such an effect.

TABLE 2
In vitro Stability of Dicarboxylate Amino Acid Prodrugs Under Conditions Prevailing in the Gut

| | Simulated gastric fluid | Simulated intestinal fluid | pH 7.4, 37° C. 0.1M |
|---|---|---|---|
| | (pH 1.1): % remaining | (pH 6.8): % remaining | phosphate buffer: % remaining |
| Compound | after 2 h/37° C. | after 2 h/37° C. | after 2 h/37° C. |
| Mexiletine lysine amide | 100 | 100 | 100 |
| Mexiletine valine amide | 98.7 | 98.3 | 98.8 |
| Mexiletine-glycocyamine | NA | NA | NA |
| amide | | | |
| Mexiletine glutamic acid | 100 | 98.3 | 100 |
| amide | | | |
| Mexiletine S-methyl- | 99.2 | 99.4 | 99.8 |
| methionine amide | | | |
| Mexiletine glycine amide | 99.8 | 99.8 | 99.9 |
| Mexiletine lysine amide | 99.8 | 80.8 | 100 |
| Mexiletine ornithine amide | 99.7 | 99.8 | 99.5 |
| Mexiletine-(S)-aspartic | 99.2 | 99.9 | 99.9 |
| acid amide | | | |
| Mexiletine (S)-N- | 99.8 | 93.6 | 99.9 |
| methylarginine amide | | | |
| Mexiletine (S)-N,N- | 99.7 | 96.8 | 99.6 |
| dimethylarginine amide | | | |

NA = Not available

### Evaluation of the Compounds

As stated earlier it is believed that the nausea and emetic activity of mexiletine arises as a direct result of a local anesthetic effect in the stomach. This is the consequence of inhibition of the slow wave movement (the “housekeeper” wave) in the stomach which facilitates stomach emptying. The local anesthetic effect of mexiletine is mediated through blockade of sodium channels. It is considered that the compounds of the present invention will reduce or eliminate emesis by having very poor activity, represented by a high IC₅₀ value against sodium channels. Thus the sodium channel blocking effect of mexiletine is temporarily inactivated by administering compounds of the invention instead of mexiletine itself. Once the compounds have been absorbed, they may be converted to mexiletine, potentially thereby providing the therapeutic benefit recognized for mexiletine with reduced or eliminated emesis and/or nausea. The IC₅₀ values shown in the following Examples demonstrate the reduced potential for emesis of the compounds shown, for example, by high IC₅₀ values in Table 4.

### Example 14

### Effects of Mexiletine and Various Mexiletine Amino Acid Prodrugs on Cloned Nav1.1 Channels Expressed in Mammalian Cells

In an attempt to identify amino acid prodrugs of mexiletine which may be (transiently) inactivated and hence less likely to have a direct emetic effect within the stomach, a series of conjugates were screened in vitro for their potential local anaesthetic activity by assessing their effects on the sodium 1.1 channel expressed in mammalian cells.

### Methods

(i) hNav1.1 Test Procedures

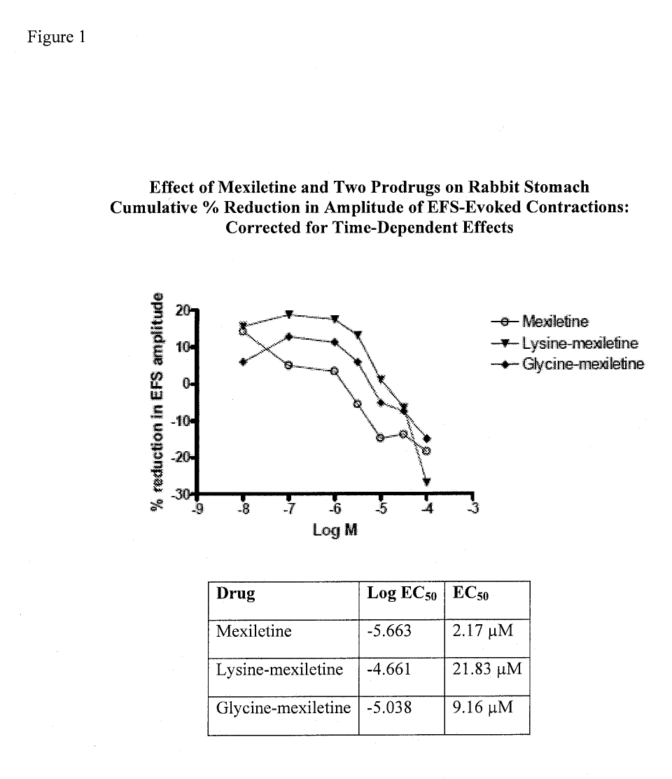

Using CHO cells stably transfected with hNav1.1 channel cDNA (SCN1A gene), the potential block of hNav1.1 channel was measured using a stimulus voltage pattern shown in FIG. 1; voltage potentials are indicated in Table 3. The pulse pattern was repeated twice: before and 5 minutes after TA addition and peak current amplitudes at three test pulses were measured (ITP1, TP11 and ITP12).

TABLE 3
Voltage-protocol parameters for hNav1.1 channel

| | Holding | Pre-Pulse | Test Pulse | Test Pulse | Interpulse | Test Pulse |
|---|---|---|---|---|---|---|
| | Potential | Potential | 1-10, 12-14 | 11 | Duration | 1-14 |
| Channel | (mV) | (mV) | Duration (ms) | Duration (ms) | (ms) | Potential (mV) |
| Nav1.1 | −80 | −120 | 20 | 500 | 80 | 0 |

### Data Analysis

Data acquisition and analyses was performed using the IonWorks Quattro™ system operation software (version 2.0.2; Molecular Devices Corporation, Union City, Calif.). Data was corrected for leak current.

The tonic block was calculated as:

% Block(Tonic)=(1−I_{TP1,TA}/I_{TP1,Control})×100%,

where I_{TP1,Control} and I_{TP1,TA} are the inward peak Na⁺ currents elicited by the TP1 in control and in the presence of a test article, respectively.

10 Hz Block—the frequency-dependent block at stimulation frequency 10 Hz was calculated as:

% Block(10 Hz)=(1−I_{TP11,TA}/I_{TP11,Control}))×100%,

where I_{TP11,Control} and I_{TP11,TA} are the inward peak Na⁺ currents elicited by the TP11 in control and in the presence of a test article, respectively.

The inactivation state block is defined as the decrease in test pulse (TP12) current amplitude due to the conditioning depolarizing pulse (TP11). The inactivation state block was calculated as:

% Block(inactivation state)=(1−(I_{TP12,TA}/I_{TP12,TA})×100%,

where I_{TP12,Control} and I_{TP12,TA} are the inward peak Na⁺ currents elicited by the TP12 in control and in the presence of a test article, respectively.
Concentration-response data for the blocks were fit to an equation of the following form:

% Block={1−1/[1+([Test]/IC₅₀)^{N}]}*100%,

where [Test] is the concentration of test article, IC₅₀ is the concentration of the test article producing half-maximal inhibition, N is the Hill coefficient, and % Block is the percentage of ion channel current inhibited at each concentration of the test article. Nonlinear least squares fits were solved with the Solver add-in for Excel 2000 (Microsoft, Redmond, Wash.).

### Results

As can be seen in Table 4, of the 45 compounds tested 13 (28%) had 1050 values in excess of 20-fold above the parent. However, there was no evident SAR and it was not predictable which compounds would demonstrate such reduced local anaesthetic activity. Such reduction in potency might be expected to reduce the potential for a direct action on the stomach/gut epithelium and resultant emesis.

TABLE 4
Summary Effects of various mexiletine prodrugs on hNav1.1 Channel

| | IC₅₀ (μM) | IC₅₀ (μM) | IC₅₀ (μM) |
|---|---|---|---|
| Compound | 10 Hz block | Tonic block | Inactivated state |
| Mexiletine (S)-tryptophan amide | 0.975 | 1.17 | 0.760 |
| Mexiletine (S)-tyrosine amide | 2.96 | 13.6 | 1.66 |
| Mexiletine methionine amide | 5.2 | 26 | 1.3 |
| Mexiletine pipecolic acid amide | 6.0 | 26 | 2.7 |
| Mexiletine dimethyl glycine amide | 10.3 | 30.5 | 3.2 |
| Mexiletine (indole-3-acetic acid) amide | >30 | >30 | 5.58 |
| Mexiletine-PHBA carbamate | 30.9 | 131 | 16.5 |
| Mexiletine 4-hydroxyproline amide | 37 | 147 | 15.5 |
| Mexiletine [(R)-S-methyl-cysteine] amide | 38.1 | 99 | 13.1 |
| Mexiletine hydrochloride | 38.2 | 115 | 9.1 |
| Mexiletine sarcosine amide | 38.9 | 186 | 6.3 |
| Mexiletine threonine amide | 41.4 | 234.5 | 11.6 |
| Mexiletine histidine amide | 42.1 | 79.5 | 21.2 |
| Mexiletine serine amide | 50.3 | 238.5 | 14.1 |
| Mexiletine 2-methyl β alanine amide | 51.8 | 137.2 | 16.3 |
| Mexiletine β alanine amide | 59.5 | 245.3 | 23.5 |
| Mexiletine-PABA amide Hydrochloride | 62.5 | 129 | 20.7 |
| Mexiletine (5-aminothiophene-2-carboxylic | 70.2 | 183 | 6.29 |
| Mexiletine glycine amide | 106.9 | 397.5 | 21.2 |
| Mexiletine (4-aminosalicylic acid) amide | 111 | 131 | 29.5 |
| Mexiletine [O-carbamoyl-(S)-serine] amide | 125 | 486 | 12.6 |
| Mexiletine glutamine amide | 138.9 | 750.5 | 67.3 |
| Mexiletine [(S)-N^{α}-acetyl-lysine] amide | 155 | 393 | 91.3 |
| Mexiletine cyclopropyl glycine amide | 169.9 | 474.8 | 42.7 |
| Mexiletine β amino alanine amide | 179.7 | 477.7 | 61.8 |
| Mexiletine [(S)-methionine sulfoxide] amide | 186 | 243 | 76.0 |
| Mexiletine [N^{α}-acetyl-(S)-ornithine] amide | 186 | 243 | 76.0 |
| Mexiletine nicotinic acid amide | 350.1 | 494.7 | 61.25 |
| Mexiletine citrulline amide | 358.6 | 491.1 | 180.5 |
| Mexiletine (urocanic acid) amide | 426 | >1000 | 252 |
| Mexiletine isonicotinic acid amide | 439.3 | 817.58 | 130.2 |
| Mexiletine-(S)-asparagine amide | 613 | >1000 | 189 |
| Mexiletine homo arginine amide | 623.6 | 742.5 | 257.5 |
| Mexiletine dihydrourocanic acid amide | 627 | >1000 | 121 |
| Mexiletine arginine amide | 811.1 | >1000 | 328.3 |
| Mexiletine [(R)-S-methyl-cysteine | 993 | >1000 | 201 |
| Mexiletine (S)-lysine amide | >1000 | 844 | 868 |
| Mexiletine [^{α}-hydroxy-(5)-valine] amide | >1000 | >1000 | 349 |
| Mexiletine-glycocyamine amide | >1000 | >1000 | 499 |
| Mexiletine glutamic acid amide | >1000 | >1000 | >1000 |
| Mexiletine S-methyl-methionine chloride | >1000 | >1000 | >1000 |
| Mexiletine (carboxymethyl-glycine) amide | >1000 | >1000 | >1000 |
| Mexiletine [(S)-N^{α}-acetyl-lysine] amide | >1000 | >1000 | >1000 |
| Mexiletine [(S)-N^{α}-acetyl-ornithine] amide | >1000 | >1000 | >1000 |
| Mexiletine-(S)-aspartic acid amide | >1000 | >1000 | >1000 |
| Mexiletine (S)-N-methylarginine amide | >1000 | >1000 | >1000 |
| Mexiletine (S)-N,N-dimemylarginine amide | >1000 | >1000 | >1000 |

### Example 15

### Synthesis of Additional Amino Acid Amide Prodrugs of Mexiletine

In addition to the forty five mexileteine amino acid amide prodrug described above, a further fifteen compounds were prepared as depicted below in Table 5.

TABLE 5
Additional amino acid amide prodrugs of mexiletine

| | |
|---|---|
| Compound | Glycine-(rac)-mexiletine Trifluoroacetate |
| Structure | 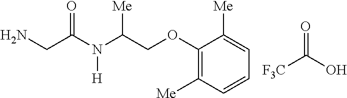 |
| NMR | 8.52 (d, J = 7.8 Hz, 1 H, NH), 8.03 (br, 3 H, NH₃⁺), 7.01 (m, |
| | 2 H, ArH), 6.93 (m, 1 H, ArH), 4.22 (m, 1 H, CH), 3.64 (m, |
| | 4 H, 2 × CH₂), 2.22 (s, 6 H, 2 × CH₃), 1.27 (d, J = 6.6 Hz, 3 |
| | H, CH₃). |
| Compound | Mexiletine-(S)-Phenylalanine Amide Hydrochloride |
| Structure | 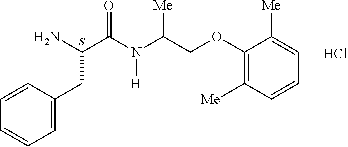 |
| NMR | 8.58 (d, J = 7.8 Hz, 0.5 H, NH), 8.47 (d, J = 7.8 Hz, 0.5 H, |
| | NH), 8.22 (br, 3 H, NH₃⁺), 7.23 (m, 5 H, 5 × Phenylalanine |
| | ArH), 7.01 (m, 2 H, 2 × ArH), 6.94 (m, 1 H, ArH), 4.16 (m, |
| | 1 H, ^{α}-CH), 4.01 (m, 1 H, CH), 3.54 (m, 2 H, CH₂), 3.05 (m, |
| | 2 H, CH₂Ph), 2.21 and 2.19 (s, 6 H, 2 × CH₃), 1.28 (d, J = |
| | 6.9 Hz, 1.5 H, ½ CH₃), 1.09 (d, J = 6.9 Hz, 1.5 H, ½ CH₃). |
| Compound | Mexiletine-(S)-Valine Amide Hydrochloride |
| Structure | 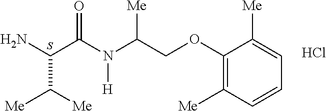 |
| NMR | 8.68 (d, J = 7.8 Hz, 1 H, NH), 8.25 (br, 3 H, NH₃⁺), 6.94 (m, |
| | 3 H, ArH), 4.23 (m, 1 H, CH), 3.62 (m, 3 H, CH + CH₂), |
| | 2.23 and 2.22 (s, 6 H, 2 × CH₃), 2.10 (m, 1 H, CH), 1.27 (d, |
| | J = 4.2 Hz, 3 H, CH₃), 0.95 (m, 6 H, 2 × CH₃). |
| Compound | Mexiletine-(S)-Ornithine Amide Di-hydrochloride |
| Structure | 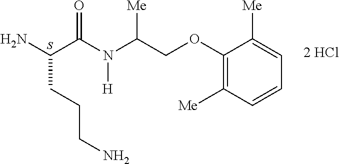 |
| NMR | 8.80 (br, 1 H, NH), 8.31 (br, 3 H, NH₃⁺), 8.01 (br, 3 H, |
| | NH₃⁺), 7.01 (m, 2 H, ArH), 6.93 (m, 1 H, ArH), 4.21 (m, |
| | 1 H, CH), 3.86-3.71 (m, 3 H, CH and CH₂), 2.79 (m, 2 H, |
| | CH₂N), 2.23 (s, 6 H, 2 × CH₃), 1.79 (m, 2 H, CH₂), 1.64 |
| | (m, 2 H, CH₂), 1.09 (m, 3 H, CH₃). |
| Compound | Mexiletine-(S)-Methionine Amide Hydrochloride |
| Structure | 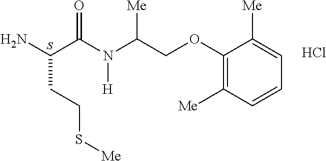 |
| NMR | 8.76 (br, 1 H, NH), 8.38 (br, 3 H, NH₃⁺), 7.01 (m, 2 H, |
| | ArH), 6.92 (m, 1 H, ArH), 4.22 (m, 1 H, CH), 3.89 (m, 1 H, |
| | CH), 3.89-3.67 (m, 2 H, CH₂), 2.50 partially hidden (m, 2 H, |
| | CH₂S), 2.22 (s, 6 H, 2 × CH₃), 2.06 m, 5 H, CH₃S and CH₂), |
| | 1.26 (m, 3 H, CH₃). |
| Compound | Mexiletine-valine-valine Amide Hydrochloride |
| Structure | 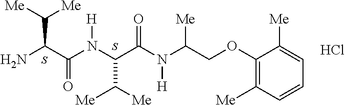 |
| NMR | 8.42 (t, J = 8.5 Hz, 1 H, NH), 8.22 (t, J = 7.9 Hz, 1 H, NH), |
| | 8.11 (br, 3 H, NH₃⁺), 7.01 (d, J = 6.8 Hz, 2 H, 2 × ArH), |
| | 6.90 (t, J = 6.4 Hz, 1 H, ArH), 4.20 (m, 2 H, α-CH and CH), |
| | 3.64 (m, 3 H, α-CH and OCH₂), 2.21 (d, J = 7.7 Hz, 6 H, 2 × |
| | CH₃), 2.00 (m, 2 H, 2 × β-CH), 1.22 (m, 3 H, CH₃), 0.90 (m, |
| | 12 H, 4 × CH₃). |
| Compound | Mexiletine-(S)-Phenylalanine-(S)-Phenylalanine Amide HCl |
| Structure | 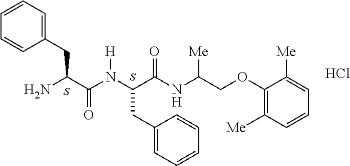 |
| NMR | 8.85 (d, J = 8.0 Hz, 1 H, NH), 8.23 (d, J = 8.0 Hz, 1 H, NH), |
| | 8.09 (br, 3 H, NH₃⁺), 7.28 (m, 10 H, ArH), 7.02 (d, J = 7.2 |
| | Hz, 2 H, ArH), 6.91 (t, J = 7.3 Hz, 1 H, ArH), 4.60 (m, 1 H, |
| | α-CH), 4.06 (b, 2 H, α-CH and CH), 3.59 (m, 2 H, OCH₂), |
| | 2.97 (m, 4 H, 2 × β-CH₂), 2.20 (s, 6 H, 2 × CH₃), 1.14 (d, J = |
| | 7.2 Hz, 3 H, CH₃). |
| Compound | Mexiletine-(S)-albizziin amide Trifluoroacetate |
| Structure | 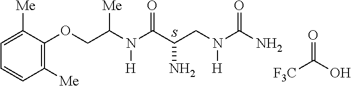 |
| NMR | 8.60 (d, J = 8.1 Hz, 0.7 H, NH), 8.55 (d, J = 8.1 Hz, 0.3 H, |
| | NH), 8.18 (br, 3 H, NH₃⁺), 7.01 (d, J = 7.5 Hz, 2 H, 2 × |
| | ArH), 6.92 (m, 1 H, ArH), 5.85 (br, 2 H, NH₂), 4.20 (m, 1 |
| | H, ^{α}-CH), 3.82 (m, 1 H, CH), 3.70 (m, 1 H, 0.5 CH₂), 3.62 |
| | (m, 1 H, 0.5 CH₂), 3.42 (m, 1 H, 0.5 CH₂), 3.33 (m, 1 H, |
| | 0.5 CH₂), 2.23 (s, 6 H, 2 × CH₃), 1.29 (m, 3 H, CH₃). |
| Compound | Mexiletine [trimethyl-(S)-lysine chloride] amide |
| | hydrochloride |
| Structure | 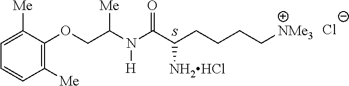 |
| NMR | 9.00 (d, J = 8.1 Hz, 0.71 H, 0.71 NH), 8.85 (d, J = 8.1 Hz, |
| | 0.26 H, 0.26 NH), 8.46 (m, 2 H, 0.67 NH₃⁺), 8.39 (m, 1 H, |
| | 0.33 NH₃⁺), 7.02 (d, J = 7.5 Hz, 2 H, ArH), 6.97 (m, 1 H, |
| | ArH), 4.20 (m, 1 H, CH), 3.77 (m, 2 H, OCH₂), 3.63-3.52 |
| | (br, 3 H, NCH₃), 3.30 (m, 1 H, CH), 3.08 (b s, 6 H, 2 × |
| | NCH₃), 2.72 (m, 2 H, CH₂), 2.23 (s, 6 H, 2 × CH₃), 1.84- |
| | 1.70 (m, 4 H, 2 × CH₂), 1.35 (m, 2 H, CH₂), 1.28 (d, J = 6.9 |
| | Hz, 3 H, CH₃). |
| Compound | Mexiletine-(S)-homoserine amide Hydrochloride |
| Structure | 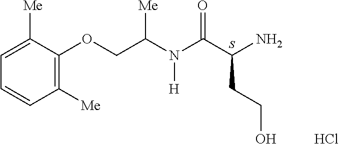 |
| NMR | 8.75 (d, J = 8.1 Hz, 1 H, NH), 8.24 (br, 3 H, NH₃⁺), 7.01 (d, |
| | J = 6.9 Hz, 2 H, 2 × ArH), 6.93 (m, 1 H, ArH), 4.19 (m, 1 H, |
| | obscured, ^{α}-CH), 3.88 (m, 1 H, CH), 3.65 (m, 2 H, CH₂), |
| | 3.52 (m, 2 H, CH₂OH), 2.22 (s, 1.5 H, 0.5 CH₃), 2.21 (s, 4.5 |
| | H, 1.5 CH₃), 1.88 (m, 2 H, CH₂), 1.28 (m, 3 H, CH₃). |
| Compound | Mexiletine-(4-Aminopiperidine-4-carboxylic acid) Amide |
| | Dihydrochloride |
| Structure | 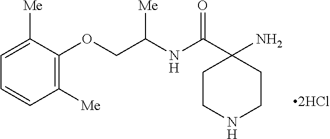 |
| NMR | 9.16 (m, 5 H, NH₂⁺ + NH₃⁺), 8.76 (d, J = 8.1 Hz, 1 H, NH), |
| | 7.02 (d, J = 7.2 Hz, 2 H, 2 × ArH), 6.91 (m, 1 H, ArH), 4.26 |
| | (m, 1 H, CH), 3.68 (m, 2 H, CH₂), 3.44 (m, 2 H, CH₂), 3.17 |
| | (m, 2 H, CH₂), 2.59 (m, 2 H, CH₂), 2.21 (s, 6 H, 2 × CH₃), |
| | 1.30 (d, J = 6.6 Hz, 3 H, CH₃) |
| Compound | Mexiletine-[N,N′-dimethyl-(S)-lysine] amide |
| | Dihydrochloride |
| Structure | 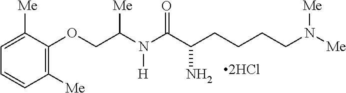 |
| NMR | 10.61 (br, 1 H, NH), 8.81 (d, J = 8.1 Hz, 1 H, NH), 8.34 (b, |
| | 3 H, NH₃⁺), 7.03 (d, J = 7.2 Hz, 2 H, 2 × ArH), 6.94 (m, 1 |
| | H, ArH), 4.22 (m, 1 H, α-CH), 3.71 (m, 3 H, CH + OCH₂), |
| | 3.00 (m, 2 H, NCH₂), 2.72 (d, J = 4.5 Hz, 3 H, NCH₃), 2.69 |
| | (d, J = 5.1 Hz, 3 H, NCH₃), 2.23 (s, 6 H, 2 × CH₃) 1.66-1.77 |
| | (m, 4 H, 2 × CH₂), 1.38 (m, 2 H, CH₂), 1.30 (d, J = 6.6 Hz, |
| | 3 H, CH₃). |
| Compound | Mexiletine lipoic acid amide |
| Structure | 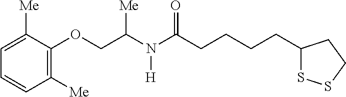 |
| NMR | 7.91 (d, J = 8.1 Hz, 1 H, NH), 7.01 (d, J = 7.5 Hz, 2 H, 2 × |
| | ArH), 6.90 (m, 1 H, ArH), 4.12 (m, 1 H, α-CH), 3.62 (m, 2 |
| | H, OCH₂), 3.14 (m, 2 H, CH₂), 2.41 (m, 1 H, CH), 2.20 (s, |
| | 6 H, 2 × CH₃), 2.09 (t, J = 7.2 Hz, 2 H, CH₂), 1.84 (m, 1 H, |
| | 0.5 CH), 1.52-1.65 (m, 5 H, 2 × CH₂ + 0.5 CH), 1.36 (m, 2 |
| | H, CH₂), 1.24 (d, J = 6.9 Hz, 3 H, CH₃. |
| Compound | Mexiletine biotin amide |
| Structure | 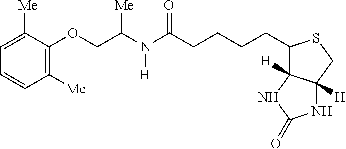 |
| NMR | 7.92 (d, J = 8.1 Hz, 1 H, NH), 7.01 (d, J = 7.2 Hz, 2 H, 2 × |
| | ArH), 6.92 (m, 1 H, ArH), 6.44 (s, 1 H, NH), 6.37 (s, 1 H, |
| | NH), 4.29 (m, 1 H, ^{α}-CH), 4.12 (m, 2 H, 2 × CH), 3.62 (m, 2 |
| | H, CH₂), 3.10 (m, 1 H, CH), 2.80 (m, 2 H, CH₂), 2.20 (s, 6 |
| | H, 2 × CH₃), 2.12 (t, 2 H, CH₂), 1.49-1.52 (m, 4 H, 2 × |
| | CH₂), 1.35 (m, 2 H, CH₂), 1.24 (d, J = 6.9 Hz, 3 H, CH₃) |
| Compound | Mexiletine ethyl carbamate amide |
| Structure | 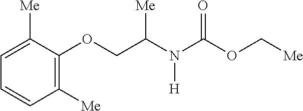 |
| NMR | 7.18 (d, J = 8.1 Hz, 1 H, NH), 7.00 (d, J = 7.5 Hz, 2 H, ArH), |
| | 6.89 (m, 1 H, ArH), 3.99 (m, 2 H, OCH₂), 3.87 (m, 1 H, |
| | CH), 3.62 (m, 2 H, OCH₂), 2.20 (s, 6 H, 2 × CH₃), 1.21 (d, |
| | J = 6.6 Hz, 3 H, CH₃), 1.16 (t, J = 6.9 Hz, 3 H, CH₃). |

### Example 16

### Evaluation of the Systemic Availability of Mexiletine in the Dog from Various Mexiletine Prodrugs

### Methods

Test substances (i.e., mexiletine, and various mexiletine amino acid prodrugs) were administered by oral gavage to groups of two or some cases five dogs. The characteristics of the test animals are set out in Table 6.

TABLE 6
Characteristics of experimental dogs used in study

| | |
|---|---|
| Species | Dog |
| Type | Beagle |
| Number and sex | 2-5 males |
| Approximate age | 4-6 months at the start of treatment |
| Approx. bodyweight | 7-9 kg at the start of treatment |

Blood samples were taken at various times after administration and submitted to analysis for parent drug using a validated LC-MS-MS assay. Pharmacokinetic parameters derived from the plasma bioanalytical data were determined using Win Nonlin. The results are given in Table 7.

### Results

The data show significant variability in the systemic availability of mexiletine from the various amino acid prodrugs tested. For example from the nictonic and isonicotinic acid amide there was negligible bioavailability with respect to mexiletine. Conversely oral administration of the glutamic acid amide or glutamine amide prodrug resulted in near complete bioavailability. Although not all forty five compounds screened for Nav 1.1 blocking activity of those those that were (21), only five gave bioavailablilities approximating (>80%) that of mexiletine itself of which three, glutamic acid amide, acetyl lysine amide, methyl methionine amide had been previously shown to have markedly reduced local anaesthetic activity (>20-fold higher IC50).

TABLE 7
Comparative pharmacokinetics of mexiletine in the dog
following oral dosing with various amino acid prodrugs
of mexiletine at 1 mg/kg mexiletine free base

| | Cmax | AUC | F(rel) | T50% |
|---|---|---|---|---|
| Compound | (ng/mL) | (ng · h/mL) | (%) | Cmax (h) |
| Mexiletine* | 138 | 1075 | 100 | 6.25 |
| Mexiletine glutamic acid | 104 | 1020 | 94.9 | 7.64 |
| amide | | | | |
| Mexiletine α acetyl | 90.8 | 893 | 83.1 | 8.08 |
| lysine amide | | | | |
| Mexiletine methyl | 121 | 869 | 80.9 | 5.92 |
| methionine amide | | | | |
| Mexiletine aminoalanine | 103 | 8371 | 77.9 | 10.0 |
| Amide | | | | |
| Mexiletine glutamine | 117 | 790 | 73.5 | 5.60 |
| amide | | | | |
| Mexiletine valine-valine | 77.2** | 565** | 52.6** | 4.25** |
| amide | | | | |
| Mexiletine arginine amide | 74.3** | 564** | 52.5** | 6.5** |
| Mexiletine homoarginine | 86.9 | 563 | 52.4 | 5.39 |
| amide | | | | |
| Mexiletine serine amide | 73.7 | 523 | 48.7 | 6.00 |
| Mexiletine glycine amide | 79.6** | 513** | 47.7** | 5.5** |
| Mexiletine phenylalanine | 72.5** | 424** | 39.5** | 3.5** |
| amide | | | | |
| Mexiletine citrulline | 93.1** | 411** | 38.2** | 3.0** |
| amide | | | | |
| Mexiletine valine amide | 69.4** | 396** | 36.8** | 5.0** |
| Mexiletine lysine amide | 54.4** | 301** | 28.0** | 5.0** |
| Mexiletine methyl- | 59.9 | 144 | 13.4 | 1.63 |
| cysteine sulfoxide amide | | | | |
| Mexiletine isonicotinic | 0.593 | NC | NC | NC |
| acid amide | | | | |
| Mexiletine nicotinic acid | BLQ | NC | NC | NC |
| amide | | | | |
| Mexiletine amino | 0.703 | NC | NC | NC |
| cyclopropylglycine | | | | |
| Mexiletine β-alanine | BLQ | NC | NC | NC |
| amide | | | | |
| Mexiletine acetyl- | BLQ | NC | NC | NC |
| ornithine amide | | | | |
| Mexiletine β-hydroxy- | 1.68 | NC | NC | NC |
| (S)-valine) Amide | | | | |

*mean of three studies
**mean of five animals
BLQ: Below limit of quantitation
NC = Not calculable
¹Calculated on AUCt.

### Example 17

### Evaluation of the Systemic Availability of Mexiletine in the Cynomolgus Monkey from Various Mexiletine Prodrugs

### Methods

Test substances (i.e., mexiletine, and various mexiletine amino acid prodrugs) were administered by oral gavage to groups of two and, in one case, five male cynomolgus monkeys. Blood samples were taken at various times after administration and submitted to analysis for the parent drug using a validated LC-MS-MS assay. Pharmacokinetic parameters derived from the plasma bioanalytical data were determined using Win Nonlin. The results are given in Table 8.

### Results

As in the dog, the data show significant variability in the systemic availability of mexiletine from the various amino acid prodrugs tested. Again although not all forty five compounds screened for Nav 1.1 blocking activity of those that were (15), four gave good relative bioavailabilities (>80%) that of mexiletine itself of which only three, glutamic acid amide, methyl methionine amide, methyl cysteine sulphoxide had been previously shown to have markedly reduced local anaesthetic activity. The acetyl lysine amide, which had shown good loss of Nav 1.1 activity had a relative bioavailability of 60%.

TABLE 8
Comparative pharmacokinetics of mexiletine in the monkey
following oral dosing with various amino acid prodrugs
of mexiletine at 1 mg/kg mexiletine free base

| | Cmax | AUC | F(rel) | T50% |
|---|---|---|---|---|
| Compound | (ng/mL) | (ng · h/mL) | (%) | Cmax (h) |
| Mexiletine* | 121 | 690 | 100 | 3.94 |
| Mexiletine aminoalanine | 82.7 | 801 | 116 | 7.88 |
| amide | | | | |
| Mexiletine glutamine | 105 | 649 | 94.1 | 4.0 |
| amide | | | | |
| Mexiletine glutamic acid | 90.6 | 604 | 87.6 | 4.76 |
| amide | | | | |
| Mexiletine methyl- | 66.9 | 583 | 84.5 | 5.99 |
| methionine amide | | | | |
| Mexiletine serine amide | 60.1 | 488 | 70.8 | 5.05 |
| Mexiletine glycine amide | 86.9** | 453** | 65.7** | 3.71** |
| Mexiletine methyl- | 62.2 | 432 | 62.6 | 4.80 |
| cysteine sulfoxide amide | | | | |
| Mexiletine homoarginine | 56.8 | 424 | 61.5 | 5.13 |
| amide | | | | |
| Mexiletine acetyl Lysine | 30.3 | 363 | 52.6 | 8.98 |
| amide | | | | |
| Mexiletine arginine | 48.9 | 340 | 49.3 | 4.48 |
| amide | | | | |
| Mexiletine isonicotinic | 1.39 | NC | NC | NC |
| acid amide | | | | |
| Mexiletine nicotinic acid | 1.04 | NC | NC | NC |
| amide | | | | |
| Mexiletine amino | 0.895 | NC | NC | NC |
| cyclopropylglycine | | | | |
| Mexiletine β-alanine | BLQ | NC | NC | NC |
| amide | | | | |
| Mexiletine (β-hydroxy- | BLQ | NC | NC | NC |
| (S)-valine) amide | | | | |

*mean of three studies (nine animals in total)
**mean of five results
BLQ Below the Limit of Quantitation (1 ng/mL)
NC = Not calculable

### Example 18

### Effects of Mexiletine and Mexiletine Glycine and Lysine Amides on Contractions of Rabbit Stomach Smooth Muscle

Using two prototypic amino acid conjugates of mexiletine (mexiletine glycine and lysine amides) with reduced sodium channel blocking potencies, the comparative direct effects of these vs mexiletine on rabbit stomach smooth muscle were examined. The magnitude of any such direct effects may be expected to be a determinant of the emesis associated with mexiletine. Reduction in any direct effects on EFS stimulated stomach smooth muscle may therefore be expected to result in a lesser emetic response.

### Methods

Strips (˜15×2 mm) of full thickness rabbit stomach smooth muscle (mucosa intact) cut from antrum area of stomach were mounted between platinum ring electrodes. The tissue was stretched to a steady tension of about 1 g and changes in force production were recorded using sensitive transducers.

Optimal voltage for stimulation was determined while the tissue was paced with an electrical field stimulation (EFS) at 14 Hz, with a pulse width of 0.5 msec. Trains of pulses then continued for 20 seconds, every 50 seconds.

EFS at optimal voltage continued throughout the protocol (stable responses=“baseline measurement of EFS”).

The test conditions employed were as follows:

(1) vehicle (deionized water, added at equivalent volume additions to test articles),
(2) Mexiletine at 7 concentrations (10 nM, 100 nM, 1 mM, 3 mM, 10 mM, 30 mM, 100 mM),
(3) Mexiletine-lysine-amide at 7 concentrations (10 nM, 100 nM, 1 mM, 3 mM, 10 mM, 30 mM, 100 mM), and
(4) Mexiletine-glycine-amide at 7 concentrations (10 nM, 100 nM, 1 mM, 3 mM, 10 mM, 30 mM, 100 mM).

Following 10 minutes of baseline EFS, the first addition of test article or vehicle (deionized water) was performed.

Test concentrations were added in a cumulative manner with PBS washes between each addition.

Test concentrations were added in a non-cumulative manner with PSS washes between each addition. Next, TTX (Na+ channel blocker) was added to the samples to confirm EFS responses were elicited via nerve stimulation, as well as to confirm activity of a sodium channel blocker (the same mechanism as mexiletine). EFS was then stopped.

### Results

The results of this investigation are clearly presented in FIG. 1 which shows a marked difference in effects of mexiletine itself compared with the prototypic amino acid prodrugs, mexiletine lysine-amide and mexiletine glycine-amide, on rabbit stomach smooth muscle. While all three compounds progressively attenuated the EFS induced contractions of rabbit stomach, the prodrug conjugates were significantly less potent in doing so. The calculated ED50 values were 2.17, 9.16, and 21.83 μM for mexiletine, mexiletine glycine amide and mexiletine lysine amide respectively. The magnitude of reduction in potency in this functional assay is consistent with that observed during the in vitro assessment of blockade of the Nav 1.1 channel and suggests the latter may be a good indicator of likely effects on the stomach epithelium. Such a reduction in the potential for direct actions on stomach muscle may minimize the likelihood of a directly mediated emetic response to the prodrug.

### Example 19

### Mexiletine and Mexiletine-Glycine-Amide—Assessment of Emetic Effects Following Oral Administration to the Ferret

Using a prototypic amino acid conjugate of mexiletine with reduced sodium channel blocking potency (mexiletine-glycine-amide) the comparative emetic effects of this versus mexiletine in the ferret were examined.

### Methods

Male ferrets (n=7) were allowed free access to pelleted diet until late afternoon on the day prior to the day of each test. The food was then removed and the ferrets were starved overnight. Food was not returned until after completion of the emetic observation. On the morning of the study, the animals were orally dosed with either 20 mg/kg mexiletine hydrochloride solution or a molar equivalent dose of mexiletine glycine amide, using a constant dose volume of 5 mL/kg. The animals were continuously observed for 2 hours post oral treatment and any incidences of retching and vomiting were recorded.

### Results

The results presented in Tables 9 and 10 show a significantly reduced frequency and duration of emesis after giving the prodrug in comparison to that seen after administering the parent compound. The average number of vomits after prodrug administration dropped to less than 30% of those observed after dosing the parent drug. Similarly, the duration of vomiting was very much reduced after prodrug administration, to less than 30% of that seen after administering mexiletine itself. Potentially, these data show a reduced ability for this prototypic mexiletine amino acid prodrug to give rise to nausea and vomiting in man, which would be expected to lead to improved efficacy and patient compliance.

TABLE 9
Effects of mexiletine and its glycine amide prodrug
on retching and vomiting in the ferret

| | | Total number of | | Time (min) to | |
|---|---|---|---|---|---|
| | | individual incidences of: | | onset of: | |
| | Animal | Retch- | Vomit- | Retch- | Vomit- |
| Treatment | no. | ing | ing | ing | ing |
| Mexiletine | 1 | 46 | 12 | 19 | 19 |
| hydro- | 2 | 9 | 3 | 30 | 30 |
| chloride | 3 | 37 | 9 | 16 | 16 |
| 20 mg/kg | 4 | 8 | 1 | 17 | 20 |
| | 5 | 30 | 11 | 15 | 15 |
| | 6 | 16 | 3 | 14 | 14 |
| | 7 | 26 | 3 | 17 | 18 |
| Mexiletine- | 1 | 20 | 5 | 15 | 17 |
| glycine-amide | 2 | 2 | 1 | 13 | 13 |
| 20 mg/kg | 3 | 12 | 2 | 11 | 11 |
| (molar | 4 | 0 | 0 | >120 | >120 |
| equivalent | 5 | 10 | 1 | 15 | 15 |
| dose to | 6 | 2 | 0 | 10 | >120 |
| mexiletine | 7 | 20 | 3 | 13 | 13 |
| HCl) | | | | | |

TABLE 10
Comparison of the effects of mexiletine hydrochloride and mexiletine
glycine amide on retching and vomiting in the ferret

| | Group mean of total number | | Group mean of duration (min) | |
|---|---|---|---|---|
| | of individual incidences of (±se): | | of total period of (±se): | |
| Treatment | Retching | Vomiting | Retching | Vomiting |
| Mexiletine hydrochloride | 24.6 ± 5.42 | 6.0 ± 1.70 | 6.7 ± 1.76 | 5.0 ± 1.27 |
| 20 mg/kg | | | | |
| Mexiletine-glycine-amide | 9.4* ± 3.20 | 1.7* ± 0.68 | 2.0* ± 0.90 | 1.3* ± 0.52 |
| 20 mg/kg | | | | |

Statistical difference from mexiletine HCl
*p < 0.05 (t test)

### Example 20

### Evaluation of the Comparative Systemic Availability of Mexiletine from the Parent Drug Versus Mexiletine Glycine Amide in the Ferret

In order to confirm that the lesser emetic effect associated with the prototypic prodrug prodrug mexiletine glycine amide was not simply the consequence of lower systemic availability of the drug a comparative pharmacokinetic study was undertaken.

### Methods

Test substances (i.e., mexiletine & mexiletine glycine amide) were administered by oral gavage to a group of six ferrets.

Blood samples were taken at various times after administration and submitted to analysis for the prodrug and parent drug using a validated LC-MS-MS assay. Pharmacokinetic parameters derived from the plasma analytical were determined using Win Nonlin.

### Results

The results are given in Table 11. Comparing systemic exposure to the drug after giving either the drug itself or the glycine amide prodrug showed a comparable overall exposure to mexiletine. As shown in Table 8 the mean relative bioavailability of mexiletine from the glycine prodrug was 94% of that after giving the parent molecule, providing confirmation that the reduced emesis associated with the prodrug was not due to poor systemic exposure to the drug

TABLE 11
Pharmacokinetics of mexiletine in the ferret after oral administration of 10 mg mexiletine
free base equivalents/kg of either mexiletine itself or mexiletine glycine amide

| Pharmacokinetic | Ferret Number | | | | | | | |
|---|---|---|---|---|---|---|---|---|
| parameter | 1 | 2 | 3 | 4 | 5 | 6 | Mean | sd |

After dosing with mexiletine

| | | | | | | | | |
|---|---|---|---|---|---|---|---|---|
| Cₘₐₓ (ng/mL) | 3770 | 2890 | 3270 | 4070 | 4420 | 2820 | 3540 | 650 |
| Tₘₐₓ (h) | 0.5 | 0.5 | 0.5 | 0.5 | 0.5 | 0.5 | 0.5^{a} | |
| AUC (ng · h/mL) | 24400 | 15600 | 18700 | 22400 | 18500 | 17500 | 19500 | 3300 |
| t 1/2 (h) | 4.0 | 3.9 | 4.6 | 4.3 | 4.6 | 6.1 | 4.5 | |

After dosing with mexiletine glycine amide

| | | | | | | | | |
|---|---|---|---|---|---|---|---|---|
| Cₘₐₓ (ng/mL) | 1890 | 1690 | 1750 | 1960 | 1840 | 1590 | 1790 | 140 |
| Tₘₐₓ (h) | 2 | 2 | 2 | 1 | 0.5 | 2 | 2^{a} | |
| AUC (ng · h/mL) | 16400 | 16700 | 19200 | 17200 | 19900 | 17800 | 17900 | 1400 |
| t½ (h) | 4.3 | 5.0 | 7.1 | 5.7 | 6.7 | 7.0 | 5.7^{b} | |
| Fᵣₑₗ(%) | 67 | 107 | 103 | 77 | 108 | 101 | 94 | |

^{a}Median value for Tₘₐₓ
^{b}Calculated as ln2/mean k

### Example 21

### Assessment of the Anti-Myotonic Effects of Mexiletine Prodrugs in the ADR Mouse

### Methods.

The homozygous ADR mouse offers a genetic model of chloride channel myotonia enabling the anti-myotonic activity of potentially active drug molecules to be assessed. Such mice show a severe phenotype, including a reduced growth rate (8 week old adr/adr mice show a body weight reduced by 50% compared to normal). The acronym ADR stands for “arrested development of righting response” and under control conditions the mean time for righting from placing these mice on their backs ranged from 3-13 seconds compared to ˜0.5 secs in wild-type mice.

The effects of oral administration of either mexiletine glutamic acid amide at 5, 10 or 20 mg/kg or mexiletine 5 mg/kg on the righting response of a group of three adr/adr mice were examined on three separate days. Each assessment involved placing the individual mice on their back no fewer than seven successive occasions in rapid succession and determining the time to successful righting at various time pre and post dosing (−10, +15, +30, +60, +120 & +180 mins).

### Results

The result are shown in Table 12 below and reveal a marked beneficial effects of dose of 10 mg/kg and above of mexiletine glutamic acid amide (MGAA).

After 5 mg/kg of either mexiletine or the prodrug the effects on righting time were but marginal. However after 10 mg/kg of MGAA the mean righting time had been reduced by almost half, from 5.18±0.43 to 2.74+0.37 secs, an effect evident within 15-60 mins but with a relatively short 1-2 h duration. After the higher dose of 20 mg/kg a mean maximum ˜60% reduction in righting time was observed which extended over 3 hours. Most importantly the variability associated with this improvement was low. These data provide a clear indication of the utility of this mexiletine prodrug in the treatment of muscle myotonia.

TABLE 12
Effects of mexiletine and mexiletine glutamic acid amide on myotonia in the ADR mouse

| | Mean* ± SEM righting response time at various times after dosing | | | | | |
|---|---|---|---|---|---|---|
| Drug/dose | −10 mins | +15 mins | +30 mins | +60 mins | +120 mins | +180 mins |
| Mex | 7.94 ± 1.25 | 7.80 ± 3.72 | 9.53 ± 5.07 | 10.5 ± 3.68 | 12.5 ± 4.98 | 12.5 ± 4.27 |
| 5 mg/kg | | | | | | |
| MGAA | 5.10 ± 0.580 | 5.70 ± 0.864 | 6.53 ± 0.928 | 7.13 ± 1.46 | 9.22 ± 1.44 | 8.38 ± 0.601 |
| 5 mg/kg | | | | | | |
| MGAA | 5.18 ± 0.427 | 3.14 ± 0.278 | 2.74 ± 0.373 | 4.41 ± 1.02 | 5.45 ± 0.529 | 6.92 ± 1.05 |
| 10 mg/kg | | | | | | |
| MGAA | 7.13 ± 1.84 | 4.23 ± 1.09 | 3.06 ± 0.884 | 4.05 ± 0.286 | 5.90 ± 0.568 | 7.90 ± 1.34 |
| 20 mg/kg | | | | | | |

*of seven successive assessments over 1 min 30 secs on three different days

### Example 22

### Assessment of the Anti-Myotonic Effects of Mexiletine Glutamic Acid Amide Verus Mexiletine in the 9-Anthracene Carboxylic Acid (9-Ac) Treated Rats

### Methods

The use of ip injection of 9-anthracene carboxylic acid is known to induce a myotonic state in rats which can then be used to assess the activity of potential anti-myotonic compounds (Villegas-NavarroA et al (1992) Exp. Toxicol. Pathol. 44 34-39). Under control conditions the righting time for male Wistar rats placed on their backs is about 0.8 secs. However 10 mins after ip injection of 30 mg/kg of 9-AC the righting time is prolonged to 1.5-4 secs.

Groups of four rats were predosed with 30 mg/kg 9-AC (time 0) and ten minutes later their righting time was assessed repeatedly seven times in rapid succession. They were then dosed po with mexiletine or mexiletine glutamic acid amide at 1, 5, 10, 20 or 40 mg/kg and the righting time reassessed at ˜30, 60, 120 and 180 minutes after 9-AC injection. At each dose level, a group of four control animals, given vehicle alone, was used

### Results

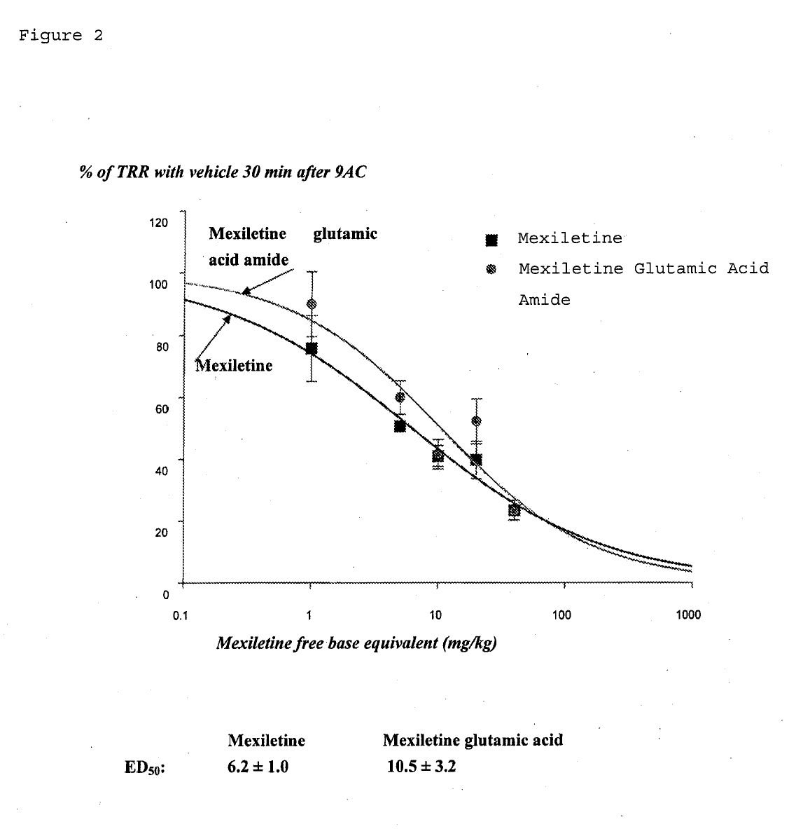

The result are shown in Table 13 below and FIG. 2 and reveal a clear beneficial effect of doses of 5 mg/kg and above of mexiletine and mexiletine glutamic acid amide (MGAA) at the time of maximal effect of 9-AC(+30 mins). Additionally, the variability associated with the beneficial response of MGAA was less than that seen after giving mexiletine itself, again at the time of maximal effect of 9-AC (+30 mins) after all doses of MGAA. For example after giving mexiletine itself at 10 mg/kg the response was associated with a coefficient of variation of 96% compared to just 43.3% after giving the prodrug at the same molar dose level. If this translated to the clinical setting, this prodrug should give rise to a more consistent and longer therapeutic response.

TABLE 13
Effects of mexiletine and mexiletine glutamic
acid amide on 9-AC induced myotonia in the rat

| | Mean¹ ± SEM righting response time (secs) at various times after dosing | | | | |
|---|---|---|---|---|---|
| | Time after 9- | | | | |
| | AC dosing + | Time after 9- | Time after 9- | Time after 9- | Time after 9- |
| | 10 mins | AC dosing + | AC dosing + | AC dosing + | AC dosing + |
| Drug/dose | (predrug) | 30 mins | 60 mins | 120 mins | 180 mins |
| Control | 2.08 ± 0.131 | 4.19 ± 0.526 | 2.27 ± 0.271 | 1.26 ± 0.180 | 0.768 ± 0.117 |
| animals | | | | | |
| Mex | 1.96 ± 0.193 | 2.94 ± 0.490 | 1.73 ± 0.165 | 1.17 ± 0.124 | 0.831 ± 0.169 |
| 1 mg/kg | | | | | |
| MGAA | 1.98 ± 0.115 | 3.52 ± 0.398 | 1.86 ± 0.195 | 1.07 ± 0.0369 | 0.714 ± 0.0427 |
| 1 mg/kg | | | | | |
| Control | 1.82 ± 0.082 | 3.39 ± 0.201 | 2.59 ± 0.243 | 1.35 ± 0.112 | 0.966 ± 0.0784 |
| animals | | | | | |
| Mex | 2.36 ± 0.208 | 2.21 ± 0.153 | 2.34 ± 0.386 | 1.33 ± 0.194 | 0.980 ± 0.220 |
| 5 mg/kg | | | | | |
| MGAA | 2.24 ± 0.208 | 2.45* 0.153 | 2.22 ± 0.0828 | 1.32 ± 0.194 | 1.11 ± 0.220 |
| 5 mg/kg | | | | | |
| Control | 2.18 ± 0.204 | 4.34 ± 0.492 | 2.81 ± 0.264 | 1.91 ± 0.144 | 1.29 ± 0.0576 |
| animals | | | | | |
| Mex | 2.26 ± 0.195 | 3.28 ± 1.58 | 2.28 ± 0.347 | 1.41 ± 0.190 | 1.03* ± 0.0348 |
| 10 mg/kg | | | | | |
| MGAA | 3.07 ± 0.754 | 2.44* ± 0.529 | 2.33 ± 0.472 | 1.26 ± 0.105 | 1.18 ± 0.156 |
| 10 mg/kg | | | | | |
| Control | 1.94 ± 0.154 | 3.48 ± 0.227 | 2.93 ± 0.733 | 1.47 ± 0.232 | 1.09 ± 0.117 |
| animals | | | | | |
| Mex | 2.27 ± 0.260 | 1.62** ± 0.310 | 1.43 ± 0.294 | 0.949 ± 0.131 | 0.871 0.113 |
| 20 mg/kg | | | | | |
| MGAA | 1.90 ± 0.274 | 1.74** ± 0.223 | 1.51 ± 0.153 | 1.18 ± 0.0973 | 1.03 ± 0.0709 |
| 20 mg/kg | | | | | |
| Control | 2.03 ± 0.338 | 4.22 ± 0.565 | 2.69 ± 0.706 | 1.28 ± 0.229 | 0.851 ± 0.179 |
| animals | | | | | |
| Mex | 2.61 ± 0.088 | 1.28* ± 0.171 | 0.913 ± 0.0507 | 0.746 ± 0.0699 | 0.583 ± 0.0606 |
| 40 mg/kg | | | | | |
| MGAA | 2.10 ± 0.077 | 1.04** ± 0.123 | 0.95 ± 0.166 | 0.669 ± 0.0558 | 0.570 ± 0.0576 |
| 40 mg/kg | | | | | |

¹mean of seven successive assessments
NB Drug administered immediately after 10 mins assessment point
* and ** indicates statistically significant difference p < 0.05 and p < 0.01 of treated versus control animals at equivalent time point

### Example 23

### Assessment to Comparative Bioavailability of Mexiletine in the Rat Following Oral Administration of Mexiletine Itself or Mexiletine Glutamic Acid Amide

In order to confirm that the observed antimyotonic activity of mexiletine glutamic acid amide in the rat (described above) was due to the systemic availability of mexiletine from its glutamic acid amide prodrug, a comparitive oral bioavailability study was undertaken.

### Methods

Test substances (i.e., mexiletine & mexiletine glutamic acid amide) were administered by oral gavage to groups of five male Sprague Dawley rats.

Blood samples were taken at various times after administration and submitted to analysis for the prodrug and parent drug using a validated LC-MS-MS assay. Pharmacokinetic parameters derived from the plasma analytical were determined using Win Nonlin.

### Results

The results are given in Tables 14-16. While the peak mexiletine plasma levels were similar after either mexiletine itself or mexiletine glutamic acid amide (MGAA), the variability after giving the prodrug was much less than after giving the drug itself. This may explain the more consistent antimyotonic response seen in the 9AC rat model described in the previous example. Furthermore there was a greater sustainment of these plasma levels as reflected by the T_{>50% Cmax} being prolonged from 1.5 to ˜4.0 h after the prodrug treatment. Associated with this sustainment of plasma drug concentrations was an increased overall bioavailability of mexiletine from the prodrug being ˜2.75-fold greater. These PK improvements should lead to greater consistency in clinical response and a longer duration of action.

TABLE 14
Pharmacokinetics of mexiletine in the rat after oral administration
of 5 mg mexiletine free base equivalents/kg of mexiletine itself

| Pharmacokinetic | | | | | | | |
|---|---|---|---|---|---|---|---|
| parameter | 1 | 2 | 3 | 4 | 5 | Mean | sd |
| Cₘₐₓ (ng/mL) | 2.68 | 7.33 | 11.0 | 5.26 | 10.2 | 7.29 | 3.45 |
| Tₘₐₓ (h) | 0.5 | 0.5 | 0.25 | 0.5 | 0.25 | 0.5^{a} | |
| AUC (ng · h/mL) | 15.9* | 15.0 | 24.0* | 10.6 | 17.4 | 14.3 | 3.4 |
| t 1/2 (h) | 4.3* | 1.2 | 1.4* | 1.2 | 1.1 | 1.2^{b} | |
| T_{>50% Cmax} (h) | 4.0 | 1.5 | 1.5 | 1.3 | 1.2 | 1.5^{a} | |

^{a}Median value
*Extrapolated AUC > 25% of total & consequently omitted from mean calculations
^{b}Calculated as ln2/mean k

TABLE 15
Pharmacokinetics of mexiletine in the rat after oral administration of 5
mg mexiletine free base equivalents/kg of mexiletine glutamic acid amide

| Pharmacokinetic | | | | | | | |
|---|---|---|---|---|---|---|---|
| parameter | 6 | 7 | 8 | 9 | 10 | Mean | sd |
| Cₘₐₓ (ng/mL) | 8.21 | 8.99 | 7.97 | 10.4 | 8.84 | 8.88 | 0.95 |
| Tₘₐₓ (h) | 1 | 1 | 2 | 2 | 1 | 1^{a} | |
| AUC (ng · h/mL) | NC | 29.2* | 33.3 | 43.4 | 40.6 | 39.1 | 5.2 |
| t 1/2 (h) | NC | 1.8* | 2.6 | 1.2 | 2.5 | 1.9^{b} | |
| T_{>50% Cmax} (h) | 4.1 | 2.5 | 2.4 | 4.1 | 3.9 | 3.9^{a} | |
| Fᵣₑₗ(%) | 163^{c} | 152^{c} | 232 | 303 | 283 | 273 | 36 |

^{a}Median value for Tₘₐₓ
*Extrapolated AUC > 25% of total & consequently omitted from mean calculations
^{b}Calculated as ln2/mean k
NC = not calculable

TABLE 16
Pharmacokinetics of mexiletine glutamic acid amide in the
rat after oral administration of 5 mg mexiletine free base
equivalents/kg of mexiletine glutamic acid amide

| Pharmacokinetic | | | | | | | |
|---|---|---|---|---|---|---|---|
| parameter | 6 | 7 | 8 | 9 | 10 | Mean | sd |
| Cₘₐₓ (ng/mL) | 127 | 186 | 101 | 180 | 138 | 146 | 36 |
| Tₘₐₓ (h) | 0.25 | 0.5 | 1.0 | 0.5 | 1.0 | 0.5^{a} | |
| AUC (ng · h/mL) | 269 | 266 | 223 | 320 | 413 | 298 | 73 |
| t 1/2 (h) | 1.0 | 0.9 | 1.9 | 0.9 | 1.3 | 1.0^{b} | |

^{a}Median value for Tₘₐₓ
^{b}Calculated as ln2/mean k

Patents, patent applications, and non-patent literature cited in herein are hereby incorporated by reference in their entirety.

1. A prodrug of mexilitine or a mexilitine analogue or a pharmaceutically acceptable salt thereof for use in the treatment of muscle myotonias and dystonias, the prodrug having a structure of Formula I: 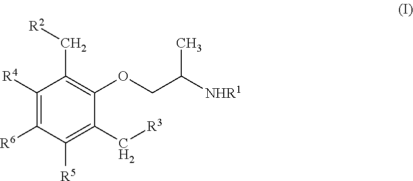
wherein
R¹ is selected from: H and a first prodrug-forming moiety selected from a group forming an amide or carbamate linkage directly to the remainder of the molecule;
each of R², R³, R⁴, R⁵ and R⁶ is independently selected from: H, OH and a second prodrug-forming moiety selected from a group forming an ester or carbamate linkage directly to the remainder of the molecule;
provided that the compound has a single prodrug moiety selected from the first and second prodrug moieties.

2. The prodrug of claim 1, wherein R¹ comprises a residue PRO¹ of a prodrug-forming moiety which, together with a carbonyl or oxy carbonyl group and the nitrogen of the adjoining NH, forms an amide or carbamate linkage between residue PRO¹ and the remainder of the molecule.

3. The prodrug of claim 1, wherein any one of R², R³, R⁴, R⁵ and R⁶ comprises a residue PRO² of a prodrug-forming moiety which, together with a carbonyloxy or an aminocarbonyloxy group, forms an ester or carbamate linkage between residue PRO² and the remainder of the molecule.

4. The prodrug of claim 2, wherein PRO₁ and PRO₂ are each an organic moiety having up 10, 20, 30, 40 or 50 multivalent atoms and further comprise at least one heteroatom selected from O and N.

5. The prodrug of claim 4, wherein PRO₁ and PRO₂ comprise a moiety selected from an amino acid, an N-substituted amino acid and a monocyclic or bicyclic ring.

6. The prodrug of claim 1, wherein the prodrug has a structure of Formula II: 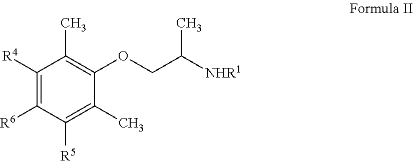
or a pharmaceutically acceptable salt thereof,
wherein,
R₁ is selected from the group consisting of: an amino acid, an amino amide residue terminating with a CONR^{g}R^{h} group, an N-substituted amino acid, a peptide having 2 to 9 amino acids, a peptide having 2 to 8 amino acids and terminating with an amino amide residue terminating with a CONR^{g}R^{h} group, an N-substituted peptide having 2 to 9 amino acids and a moiety having the structure: 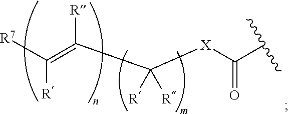
wherein,
m is 0, 1, 2, 3 or 4;
n is 0 or 1;
X is a bond or —O—;
R′ and R″ are each independently selected from the group consisting of: H, hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; and
R⁷ is selected from the group consisting of: H, substituted or unsubsititued aryl and substituted or unsubsititued heterocycle (e.g. substituted or unsubstituted heteroaryl) wherein the substituted aryl and substituted heterocycle (e.g. substituted heteroaryl) groups have 1, 2 or 3 substituents independently selected from the group consisting of: hydroxy, carboxy, oxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; and
R^{g} and R^{h} when present are each independently selected from the group consisting of: H, C₁₋₆ alkyl, —(CH₂)ₛ—C₃₋₆ cycloalkyl, phenyl and benzyl, or wherein R^{g} and R^{h} together with the nitrogen atom to which they are attached form a ring containing 3, 4, 5 or 6 carbon atoms; wherein each of the R^{g} and R^{h} groups may be unsubstituted or substituted with 1 or 2 substituent groups independently selected at each occurrence from the group consisting of: F, Cl, CN and OH;
s is an integer of 0 or 1;
R⁴, R⁵ and R⁶ are each independently selected from hydrogen and [symbol] OH.

7. The prodrug of claim 6, wherein R⁴, R⁵ and R⁶ are each hydrogen.

8. The prodrug of claim 6, wherein R¹ is an amino acid.

9. The prodrug of claim 6, wherein R¹ is an N-substituted amino acid.

10. The prodrug of claim 6, wherein R¹ is an amino amide residue terminating with a CONR^{g}R^{h} group.

11. The prodrug of claim 6, wherein R¹ is a peptide having 2 to 8 amino acids and terminating with an amino amide residue terminating with a CONR^{g}R^{h} group, wherein optionally R¹ is a peptide of 1 to 2 independently selected amino acids and terminating with an amino amide residue terminating with a CONR^{g}R^{h} group.

12. The prodrug of claim 10, wherein R^{g} is selected from the group consisting of: H, Me, Et and cyclopropyl, optionally R^{g} is H.

13. The prodrug of claim 10, wherein R^{h} is selected from the group consisting of: H, Me, Et and cyclopropyl, optionally R^{h} is H.

14. The prodrug of claim 6, wherein R¹ is a peptide of 2 to 9 independently selected amino acids, wherein optionally R¹ is a peptide of 2 to 3 independently selected amino acids.

15. The prodrug of claim 6, wherein R¹ is an N-substituted peptide of 2 to 9 independently selected amino acids, wherein optionally 2 to 3 independently selected amino acids.

16. The prodrug of claim 6, wherein R¹ is 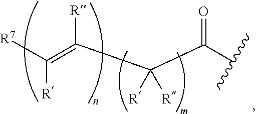
optionally wherein R′ and R″ are each H.

17. The prodrug of claim 16, wherein n is 0 and m is 0.

18. The prodrug of claim 16, wherein n is 1 and m is 0.

19. The prodrug of claim 16, wherein n is 0 and m is 1.

20. The prodrug of claim 16, wherein n is 0 and m is 2.

21. The prodrug of claim 16, wherein n is 0 and m is 4.

22. The prodrug of claim 16, wherein R⁷ is substituted or unsubsititued aryl, optionally substituted or unsubsititued phenyl, further optionally 4-hydroxy phenyl, 4-amino phenyl or 4-aminosalicylic acid.

23. The prodrug of claim 16, wherein R⁷ is unsubsititued heteroaryl, optionally R⁷ is 3-pyridyl, 4-pyridyl, 5-aminothiophen-2-carboxylic acid, unsubsititued 3-indoly or unsubsititued 5-imidazolyl.

24. The prodrug of claim 16, wherein R⁷ is substituted or unsubstituted heterocyclyl, optionally R⁷ is 1,2-dithiolan-3-yl or

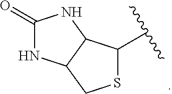

25. The prodrug claim 1 wherein the prodrug is mexilitine glutamic acid amide, mexiletine aspartic acid amide, mexiletine S-methyl-methionine chloride amide, mexiletine [(S)—N^{α}-acetyl-lysine] amide, mexiletine[(R)—S-methylcysteine sulphoxide amide, mexiletine homoarginine amide, mexiletine (carboxymethyl-glycine) amide, mexiletine-glycocyamine amide, mexiletine (S)—N-methylarginine amide or mexiletine (S)—N,N-dimethylarginine amide.

26. A compound selected from the group consisting of: mexiletine-N-methylarginine amide, mexiletine-N,N-dimethylarginine amide, Mexiletine tryptophan amide, Mexiletine tyrosine amide, Mexiletine (indole-3-acetic acid) amide, Mexiletine-PHBA carbamate, Mexiletine [S-methyl-cysteine] amide, Mexiletine-PABA amide, Mexiletine (5-aminothiophene-2-carboxylic acid) amide, Mexiletine (4-aminosalicylic acid) amide, Mexiletine [O-carbamoyl-serine] amide, Mexiletine [N-acetyl-lysine] amide, Mexiletine [methionine sulfoxide] amide, Mexiletine [N^{α}-acetyl-ornithine] amide, Mexiletine (urocanic acid) amide, Mexiletine dihydrourocanic acid amide, Mexiletine [S-methyl-cysteine sulfoxide] amide, Mexiletine [β-hydroxy-valine] amide, Mexiletine-glycocyamine amide, Mexiletine (carboxymethyl-glycine) amide, Mexiletine [N^{α}-acetyl-lysine] amide, Mexiletine [N^{ε}-acetyl-ornithine] amide, Mexiletine-aspartic acid amide, Mexiletine-Valine Amide, Mexiletine-Ornithine Amide, Mexiletine-valine-valine Amide, Mexiletine-Phenylalanine-Phenylalanine Amide, Mexiletine-albizziin amide, Mexiletine [trimethyl-lysine chloride] amide, Mexiletine-homoserine amide, Mexiletine-(4 Aminopiperidine-4-carboxylic acid) Amide, Mexiletine-[N,N′-dimethyl-lysine] amide, Mexiletine lipoic acid amide, Mexiletine biotin amide and Mexiletine ethyl carbamate amide.

27. The compound of claim 26 for use as a medicament.

28. The compound of claim 26 for use in the treatment of myotonic conditions (e.g. neuropathic myotonic conditions) or dystonic conditions.

29. A pharmaceutical composition of the mexiletine prodrug comprising a compound of claim 1, or a pharmaceutically acceptable salt thereof, and at least one pharmaceutically acceptable excipient.

30. A mexilitine prodrug for use in the treatment of muscle myotonias and dystonias, the prodrug having a structure according to Formula (III): 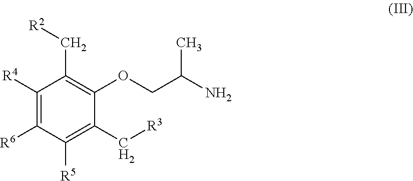
or a pharmaceutically acceptable salt thereof, wherein:
one of R², R³, R⁴, R⁵ and R⁶ is: 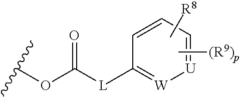
and the rest of R², R³, R⁴, R⁵ and R⁶ are each H;
L is a bond or is a linker moiety e.g. comprising a linear chain having a length of from 1 to 20 atoms;
wherein R⁸ is selected from the group consisting of: —(CR′R″)ᵣCOOH, —(CR′R″)ᵣCOOR^{g}, —(CR′R″)ᵣCONR^{g}R^{h}, 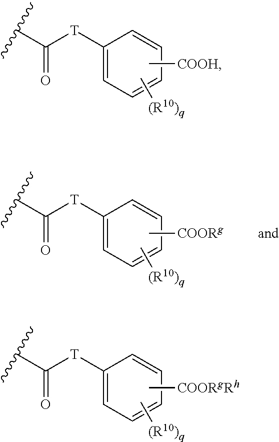
wherein T is —O— or —NR¹¹—; wherein R′ and R″ are each independently selected from the group consisting of: H, hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; wherein R^{g} and R^{h} when present are each independently selected from the group consisting of: H, C₁₋₆ alkyl, —(CH₂)ₛ—C₃₋₆ cycloalkyl, phenyl and benzyl, or wherein R^{g} and R^{h} together with the nitrogen atom to which they are attached form a ring containing 3, 4, 5 or 6 carbon atoms; wherein each of the R^{g} and R^{h} groups may be unsubstituted or substituted with 1 or 2 substituent groups independently selected at each occurrence from the group consisting of: F, Cl, CN and OH; and wherein s is an integer of 0 or 1;
R¹¹ is selected from the group consisting of: H, C₁₋₄ alkyl (e.g. methyl, ethyl or propyl), C₁₋₄ haloalkyl (e.g. trifluoromethyl), C₁₋₄ alkoxy (e.g. methoxy, ethoxy or propoxy), C₁₋₄ haloalkoxy (e.g. trifluoromethoxy);
R⁹ and R¹⁰ are each independently selected from the group consisting of: hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;
W and U are each independently selected from the group consisting of: —CR′═ and —N═;
p is 0, 1 or 2;
q is 0, 1 or 2; and
r is 0, 1 or 2;
wherein each moiety R′ is independently selected from the others.

31. A prodrug having a structure of Formula II: 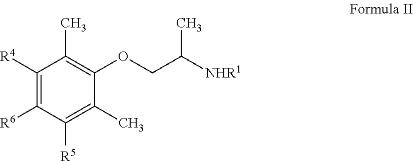
or a pharmaceutically acceptable salt thereof,
wherein,
R₁ is selected from the group consisting of: an amino amide residue terminating with a CONR^{g}R^{h} group, a peptide having 2 to 8 amino acids and terminating with an amino amide residue terminating with a CONR^{g}R^{h} group and a moiety having the structure: 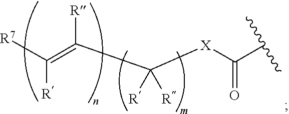
wherein,
m is 0, 1, 2, 3 or 4;
n is 0 or 1;
X is a bond or —O—;
R′ and R″ are each independently selected from the group consisting of: H, hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; and
R⁷ is selected from the group consisting of: substituted aryl and substituted heterocycle (e.g. substituted heteroaryl) wherein the substituted aryl and substituted heterocycle (e.g. substituted heteroaryl) groups have 1, 2 or 3 substituents independently selected from the group consisting of: COOR^{g}, provided that —COOR^{g} is not —COOH, and CONR^{g}R^{h};
R^{g} and R^{h} are each independently selected from the group consisting of: H, C₁₋₆ alkyl, —(CH₂)ₛ—C₃₋₆ cycloalkyl, phenyl and benzyl, or wherein R^{g} and R^{h} together with the nitrogen atom to which they are attached form a ring containing 3, 4, 5 or 6 carbon atoms; wherein each of the R^{g} and R^{h} groups may be unsubstituted or substituted with 1 or 2 substituent groups independently selected at each occurrence from the group consisting of: F, Cl, CN and OH;
s is an integer of 0 or 1;
R⁴, R⁵ and R⁶ are each independently selected from hydrogen and [symbol] OH.

32. A mexilitine prodrug the prodrug having a structure according to Formula (III): 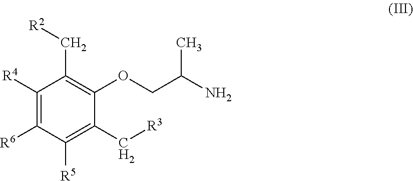
or a pharmaceutically acceptable salt thereof, wherein:
one of R², R³, R⁴, R⁵ and R⁶ is: 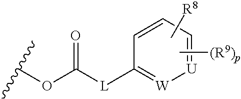
and the rest of R², R³, R⁴, R⁵ and R⁶ are each H;
L is a bond or is a linker moiety e.g. comprising a linear chain having a length of from 1 to 20 atoms (e.g. 1 to 10 atoms);
wherein R⁸ is selected from the group consisting of: —(CR′R″)ᵣCOOR^{g}, provided that —(CR′R″)ᵣCOOR^{g} is not —COOH, —(CR′R″)ᵣCONR^{g}R^{h}, 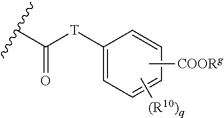
provided that when q is zero, —COOR^{g} is not —COOH, and 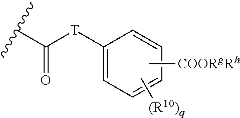
wherein T is —O— or —NR¹¹—; wherein R′ and R″ are each independently selected from the group consisting of: H, hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl; wherein R^{g} and R^{h} when present are each independently selected from the group consisting of: H, C₁₋₆ alkyl, —(CH₂)ₛ—C₃₋₆ cycloalkyl, phenyl and benzyl, or wherein R^{g} and R^{h} together with the nitrogen atom to which they are attached form a ring containing 3, 4, 5 or 6 carbon atoms; wherein each of the R^{g} and R^{h} groups may be unsubstituted or substituted with 1 or 2 substituent groups independently selected at each occurrence from the group consisting of: F, Cl, CN and OH; and wherein s is an integer of 0 or 1;
R¹¹ is selected from the group consisting of: H, C₁₋₄ alkyl (e.g. methyl, ethyl or propyl), C₁₋₄ haloalkyl (e.g. trifluoromethyl), C₁₋₄ alkoxy (e.g. methoxy, ethoxy or propoxy), C₁₋₄ haloalkoxy (e.g. trifluoromethoxy);
R⁹ and R¹⁰ are each independently selected from the group consisting of: hydroxy, carboxy, carboxamido, imino, alkanoyl, cyano, cyanomethyl, nitro, amino, substituted amino, halogen (e.g. fluoro, chloro or bromo), C₁₋₆ alkyl (e.g. methyl, ethyl or propyl), C₁₋₆ haloalkyl (e.g. trifluoromethyl), C₁₋₆ alkoxy (e.g. methoxy, ethoxy or propoxy), C₁₋₆ haloalkoxy (e.g. trifluoromethoxy), C₃₋₆ cycloalkyl (e.g. cyclopropyl or cyclohexyl), aryl (e.g. phenyl), aryl-C₁₋₆ alkyl (e.g. benzyl) and C₁₋₆ alkyl aryl;
W and U are each independently selected from the group consisting of: —CR′═ and —N═;
p is 0, 1 or 2;
q is 0, 1 or 2; and
r is 0, 1 or 2;
wherein each moiety R′ is independently selected from the others.
